(19) United States
(12) Patent Application Publication
(10) Pub. No.: US 2011/0166128 A1
(43) Pub. Date: Jul. 7, 2011

(54) Diaryldiazepine Prodrugs for the Treatment of Neurological and Psychological Disorders

(75) Inventors: Julius F. Remenar, Framingham, MA (US); Laura Cook Blumberg, Lincoln, MA (US)

(73) Assignee: Alkermes, Inc., Waltham, MA (US)

(21) Appl. No.: 12/978,240

(22) Filed: Dec. 23, 2010

Related U.S. Application Data
(60) Provisional application No. 61/293,163, filed on Jan. 7, 2010.
(60) Provisional application No. 61/293,124, filed on Jan. 7, 2010.

(51) Int. Cl. A61K 31/55; C07D 495/04; A61P 25/18; C07D 403/04; A61P 25/00

(52) U.S. Cl. 514/220; 540/557

(57) ABSTRACT

The present invention provides prodrug compounds of diaryldiazepine drug compounds.

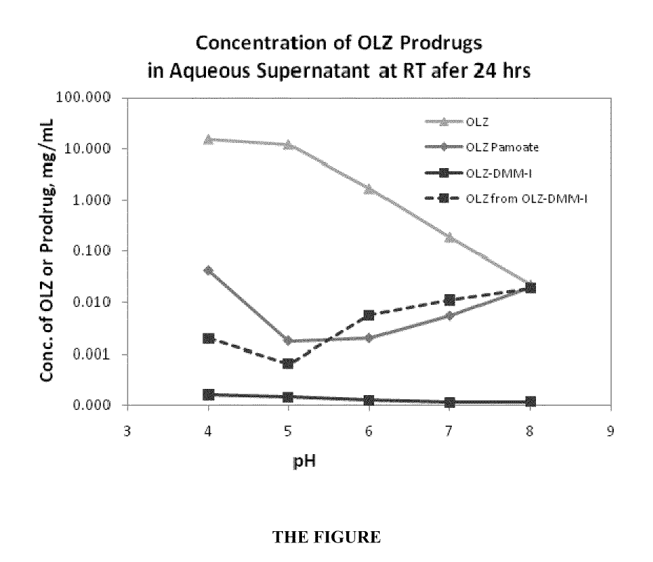

### RELATED APPLICATION(S)

This application claims the benefit of U.S. Provisional Application Nos. 61/293,163 and 61/293,124, both filed on Jan. 7, 2010. The entire teachings of the above application(s) are incorporated herein by reference.

### BACKGROUND OF THE INVENTION

(i) Field of the Invention

The present invention relates to prodrugs of diaryldiazepine drugs.

(ii) Background of the Invention

Drug delivery systems are often critical for the safe effective administration of a biologically active agent. Perhaps the importance of these systems is best realized when patient compliance and consistent dosing are taken into consideration. For instance, reducing the dosing requirement for a drug from four-times-a-day to a single dose per day would have significant value in terms of ensuring patient compliance and optimizing therapy.

Optimization of a drug's bioavailability has many potential benefits. For patient convenience and enhanced compliance it is generally recognized that less frequent dosing is desirable. By extending the period through which the drug is released, a longer duration of action per dose is expected. This will then lead to an overall improvement of dosing parameters such as taking a drug once a day where it has previously required four times a day dosing or once a week or even less frequently when daily dosing was previously required. Many drugs are presently given at a once a day dosing frequency. Yet, not all of these drugs have pharmacokinetic properties that are suitable for dosing intervals of exactly twenty-four hours. Extending the period through which these drugs are released would also be beneficial.

One of the fundamental considerations in drug therapy involves the relationship between blood levels and therapeutic activity. For most drugs, it is of primary importance that serum levels remain between a minimally effective concentration and a potentially toxic level. In pharmacokinetic terms, the peaks and troughs of a drug's blood levels ideally fit well within the therapeutic window of serum concentrations. For certain therapeutic agents, this window is so narrow that dosage formulation becomes critical.

In an attempt to address the need for improved bioavailability, several drug release modulation technologies have been developed. Enteric coatings have been used as a protector of pharmaceuticals in the stomach and microencapsulating active agents using protenoid microspheres, liposomes or polysaccharides has been effective in abating enzyme degradation of the active agent. Enzyme inhibiting adjuvants have also been used to prevent enzymatic degradation.

A wide range of pharmaceutical formulations provide sustained release through microencapsulation of the active agent in amides of dicarboxylic acids, modified amino acids or thermally condensed amino acids. Slow release rendering additives can also be intermixed with a large array of active agents in tablet formulations.

While microencapsulation and enteric coating technologies impart enhanced stability and time-release properties to active agent substances these technologies suffer from several shortcomings. Incorporation of the active agent is often dependent on diffusion into the microencapsulating matrix, which may not be quantitative and may complicate dosage reproducibility. In addition, encapsulated drugs rely on diffusion out of the matrix or degradation of the matrix, or both, which is highly dependent on the chemical properties and water solubility of the active agent. Conversely, water-soluble microspheres swell by an infinite degree and, unfortunately, may release the active agent in bursts with limited active agent available for sustained release. Furthermore, in some technologies, control of the degradation process required for active agent release is unreliable. For example, an enterically coated active agent depends on pH to release the active agent and, due to variability in pH and residence time, it is difficult to control the rate of release.

Several implantable drug delivery systems have utilized polypeptide attachment to drugs. Additionally, other large polymeric carriers incorporating drugs into their matrices are used as implants for the gradual release of drug. Yet another technology combines the advantages of covalent drug attachment with liposome formation where the active ingredient is attached to highly ordered lipid films.

However there is still a need for an active agent delivery system that is able to deliver certain active agents which have been heretofore not formulated or difficult to formulate in a sustained release formulation for release over a sustained period of time and which is convenient for patient dosing.

Currently there are several drugs in the diaryldiazepine class in clinical use for the treatment of neurological and psychological disorders including schizophrenia and bipolar disorder. Examples of these compounds include the atypical antipsychotics olanzapine and clozapine, the structures of which are shown below.

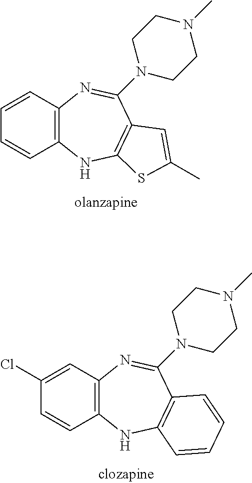

Other examples of diaryldiazepine derivatives reported to be useful for the treatment of psychological disorders are disclosed in U.S. Pat. Nos. 4,097,597; 4,096,261; 4,087,421; 3,956,297; 3,951,981; 3,903,105; Published PCT Application WO 95/17400 and European Patent EP 54416.

Thus far there have been no prodrugs of diaryldiazepine drugs that provide sustained release or zero order kinetics by, for example, decreasing the solubility of the parent drug. There is a generally recognized need for sustained delivery of anti-psychotic drugs that reduces the daily dosing requirement and allows for controlled and sustained release of the parent diaryldiazepine drug and also avoids irregularities of release and cumbersome formulations encountered with typical dissolution controlled sustained release methods.

### SUMMARY OF THE INVENTION

The present invention accomplishes this by extending the period during which a diaryldiazepine parent drug is released and absorbed after administration to the patient and providing a longer duration of action per dose than is currently expected. In one embodiment, the compounds suitable for use in the methods of the invention are labile prodrugs of diaryldiazepine parent drugs that are derivatized through aldehyde linked prodrug moieties that reduce the solubility and polarity of the prodrug compound as compared to the parent drug.

It is understood that any of the diaryldiazepine parent drugs and diaryldiazepine parent prodrugs of the invention may be further substituted as that term is defined herein so long as the substituted parent drug or parent prodrug, which when administered to a patient in vivo, becomes cleaved by chemical and/or enzymatic hydrolysis thereby releasing the parent drug moiety such that a sufficient amount of the compound intended to be delivered to the patient is available for its intended therapeutic use in a sustained release manner. One example of a substituted diaryldiazepine-containing parent drug or a prodrug comprising a substituted diaryldiazepine-containing parent drug is a pharmaceutically acceptable ester of the diaryldiazepine-containing parent drug. A parent drug or parent prodrug may be further substituted for any purpose including, but not limited to, stabilization of the parent during synthesis of the prodrug and stabilization of the prodrug for administration to the patient.

In one embodiment, the invention provides compounds represented by Formula I or Formula II,

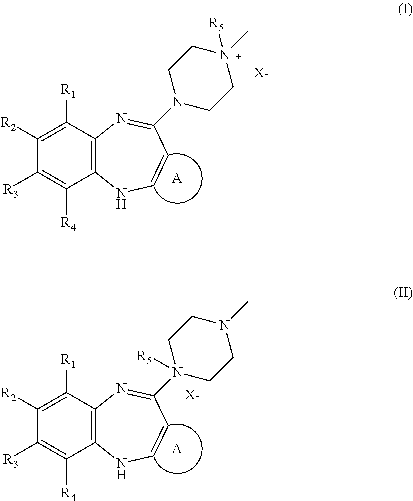

wherein:

Ring A is a fused aryl or heteroaryl ring, each optionally substituted;

each of R₁ to R₄ is independently selected from hydrogen, halogen, —OR₆, —SR₆, —N(R₆)(R₇), optionally substituted aliphatic, optionally substituted aryl, optionally substituted heteroaryl and optionally substituted heterocyclyl;

R₅ is selected from —C(R₈)(R₉)—OC(O)OR₁₀, —C(R₈)(R₉)—OC(O)R₁₀, —C(R₈)(R₉)—OC(O)NR₁₁R₁₂, —C(R₈)(R₉)—OPO₃MY, —C(R₈)(R₉)—OP(O)(OR₁₁)(OR₁₂), and —CH(R₈)(R₉)—OP(O)₂(OR₁₁)M;

R₆ and R₇ are each independently C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₃-C₁₂ cycloalkyl, substituted C₃-C₁₂-cycloalkyl; or

R₆, R₇ and the nitrogen atom to which they are attached form a substituted or unsubstituted heterocyclic ring;

R₈ and R₉ are each independently hydrogen, aliphatic or substituted aliphatic.

In one embodiment, R₁₀, or at least one of R₁₁ and R₁₂, is an optionally substituted aliphatic pr aromatic group that reduces the solubility of the prodrug ubder physiological conditions compared to the parent drug.

R₁₀ is preferably C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, aryl or substituted aryl;

R₁₁ and R₁₂ are each independently hydrogen, aliphatic or substituted aliphatic, provided that at least one of R₁₁ and R₁₂ is C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₂₄ cycloalkyl, substituted C₃-C₁₂-cycloalkyl;

X⁻ is a pharmaceutically acceptable anion; and

Y and M are the same or different and each is a monovalent cation; or M and Y together are a divalent cation;

or a pharmaceutically acceptable salt thereof.

It is to be understood that in compounds of Formula I and II in which R₅ is —C(R₈)(R₉)—OPO₃MY or —CH(R₈)(R₉)—OP(O)₂(OR₁₁)M, it is possible for the phosphate moiety to serve as X— and for the quaternary ammonium group to serve as M.

In another embodiment, the invention provides a compound of Formula III or Formula IV:

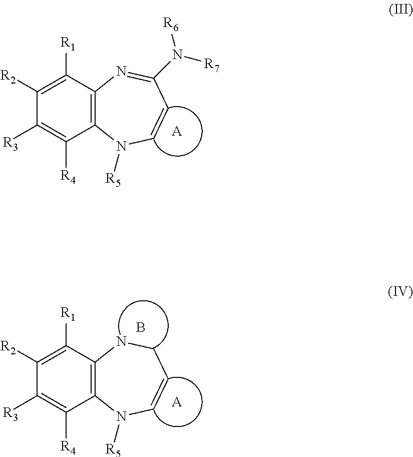

wherein R₅ is selected from —C(R₈)(R₉)—OC(O)OR₁₀, —C(R₈)(R₉)—OC(O)R₁₀, —C(R₈)(R₉)—OC(O)NR₁₁R₁₂, —C(R₈)(R₉)—OPO₃MY, —C(R₈)(R₉)—OP(O)(OR₁₁)(OR₁₂), —CH(R₈)(R₉)—OP(O)₂(OR₁₁)M, —[C(R₈)(R₉)O]ₙ—C(O)OR₁₀, —[C(R₈)(R₉)O]ₙ—C(O)R₁₀, —[C(R₈)(R₉)O]ₙ—C(O)NR₁₁R₁₂, —[C(R₈)(R₉)O]ₙ—PO₃MY, —[C(R₈)(R₉)O]ₙ—P(O)₂(OR₁₁)M and —[C(R₈)(R₉)O]ₙ—P(O)(OR₁₁)(OR₁₂); n is 2 or 3; Ring B is a fused heteroaryl ring; and R₁-R₄, R₆, R₇, R₈, R₉, R₁₀, R₁₁, R₁₂ and Ring A have the identities given for these variables in Formula I.

In another embodiment, the invention relates to pharmaceutical compositions comprising a compound of Formula I, Formula II, Formula III or Formula IV and a pharmaceutically acceptable carrier.

In one embodiment, the prodrug compounds of the invention are formulated with a biocompatible sustained release delivery system for delivery of the prodrug wherein the system is preferably capable of minimizing accelerated hydrolytic cleavage of the prodrug by minimizing exposure of the prodrug to water. Preferred delivery systems include biocompatible polymeric matrix delivery systems capable of minimizing the diffusion of water into the matrix having the prodrug dispersed therein.

In yet another embodiment, the invention relates to a method of treating a psychiatric disorder in a subject in need thereof, comprising administering to the subject a therapeutically effective amount of a compound of Formula I, Formula II, Formula III or Formula IV.

### BRIEF DESCRIPTION OF THE DRAWINGS

The FIGURE is a graph illustrating the pH dependence of the solubility of olanzapine free base, olanzapine pamoate salt and Compound 18.

### DETAILED DESCRIPTION OF THE INVENTION

In one embodiment, the compounds of the invention include compounds which are derivatives of a parent diaryldiazepine drug compound and have lower solubility than the parent compound under physiological conditions, such as physiological pH and ionic strength. Preferably a compound of the invention converts to its parent compound in vivo. In some embodiments, this conversion involves an enzymatic process. In other embodiments, the conversion does not involve an enzymatic process.

In one embodiment, the invention provides compounds of Formulas I to IV in which Ring A is benzo or a fused heteroaromatic ring, such as a thiophene, pyridine, pyrazole, diazole, triazole, oxazole, or thiazole ring. In preferred embodiments, Ring A is benzo or thiophene.

In one embodiment, the invention provides compounds of Formulas I to IV in which each of R₁, R₂, R₃ and R₄ is hydrogen. In another embodiment R₁, R₃ and R₄ are each hydrogen, and R₂ is chlorine.

In a preferred embodiment, R₉ is hydrogen and R₈ is selected from the group consisting of hydrogen; C₁-C₃-alkyl, preferably methyl or isopropyl; —C(O)H, —CH(OH)CH₂OH, —C(O)OH or —C(O)OEt. In a particularly preferred embodiment, both R₈ and R₉ are hydrogen.

In one embodiment, the invention provides compounds of Formulas I to IV in which R₅ is selected from —CH(R₈)—OC(O)OR₁₀, —CH(R₈)—OC(O)R₁₀ and —CH(R₈)—OC(O)NR₁₁R₁₂. In another embodiment, R₅ is selected from —CH(R₈)—OPO₃MY and —CH(R₈)—OP(O)(OR₁₁)(OR₁₂).

In one embodiment, R₁₀, or at least one of R₁₁ and R₁₂, is optionally substituted C₇-C₂₄-alkyl, C₇-C₂₄-alkenyl, or C₇-C₂₄-alkynyl. In another embodiment, R₁₀, or at least one of R₁₁ and R₁₂, is branched C₃-C₂₄-alkyl, -alkenyl or -alkynyl, preferably branched C₇-C₂₄ alkyl, -alkenyl or -alkynyl. In one embodiment, R₁₀, or at least one of R₁₁ and R₁₂, is a secondary or tertiary C₃-C₂₄- or C₇-C₂₄-alkyl, -alkenyl or -alkynyl group, such as C₃-C₁₂-cycloalkyl, 1-methyl-C₃-C₁₂-cycloalkyl, isopropyl, sec-butyl, t-butyl, pent-2-yl, hex-2-yl, hept-2-yl, cyclopentyl, neopentyl, 3-methylpent-3-yl, 3-ethylpent-3-yl; 2,3-dimethylbut-2-yl; 1-methylcyclopentyl, 1-methylcyclohexyl, 1-ethylcyclohexyl or a branched alkyl group corresponding to one of formulas (i)-(v) below.

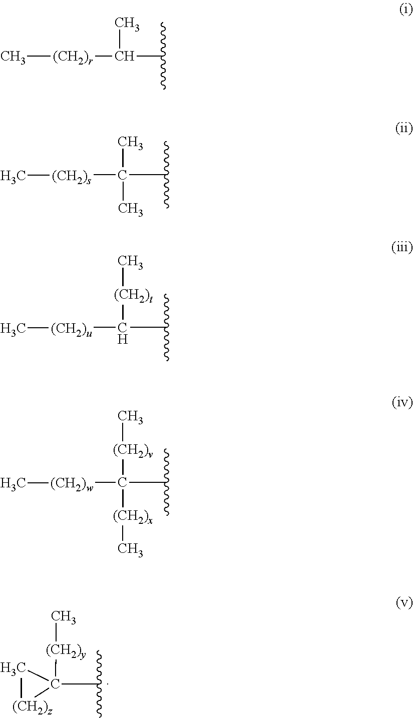

In these groups, r is 0 to 21 and s is 0 to 20. Each of t and u is independently 0 to 21, provided that the sum of t and u is from 0 to 21. Each of v, w and x is independently 0 to 20, provided that the sum of v, w and x is from 0 to 20. z is an integer from 1 to 10 and y is an integer from 0 to 20, provided that the sum of z and y is from 1 to 21. Preferably, r is an integer from 5 to 21; s is an integer from 2 to 20; the sum of t and u is from 5 to 21; the sum of v, w and x is from 4 to 20; and the sum of y and z is from 5 to 21. R₁₀ can also be an alkenyl or alkynyl group derived from one of the alkyl groups of formulas (i) to (v), by replacement of one or more carbon-carbon single bonds with a carbon-carbon double bond or a carbon-carbon triple bond.

In another embodiment, R₁₀, or at least one of R₁₁ and R₁₂, is an optionally substituted β-branched C₄-C₂₄-alkyl, C₄-C₂₄-alkenyl or C₄-C₂₄-alkynyl, preferably optionally substituted β-branched C₇-C₂₄-alkyl, C₇-C₂₄-alkenyl or C₇-C₂₄-alkynyl. Suitable examples of β-branched alkyl groups include 2-methyl-C₃-C₂₃-alkyl and 2,2-dimethyl-C₃-C₂₂-alkyl, including 2-methylpropyl; 2,2-dimethylpropyl; 2-methylbutyl; 2,2-dimethylbutyl; 2-methylpentyl; 2,2-dimethylpentyl; and 2-ethyl-2-methylbutyl.

In another embodiment, R₁₀, or at least one of R₁₁ and R₁₂, is an optionally substituted aryl-C₁-C₂₄-alkyl group, such as a phenyl-C₁-C₂₄-alkyl group. In another embodiment, R₁₀, or at least one of R₁₁ and R₁₂, is optionally substituted C₁-C₂₄-alkylaryl, optionally substituted C₁-C₂₄-alkenylaryl or optionally substituted C₁-C₂₄-alkynylaryl.

In one embodiment, the invention provides compounds of Formulas I to IV in which R₅ is —CH(R₈)—OPO₃MY, where M and Y are each independently a monovalent cation, such as H⁺, Na⁺, K⁺, NH₄⁺, Cs⁺, or an organic cation such as an organic ammonium ion or a guanadinium ion. M and Y can also together represent a divalent cation, such as a Zn²⁺, Fe²⁺, Ca²⁺, or Mg²⁺. Preferably, M and Y together are Ca²⁺.

In certain embodiments, R₅ is a group defined by one of the structures set forth below.

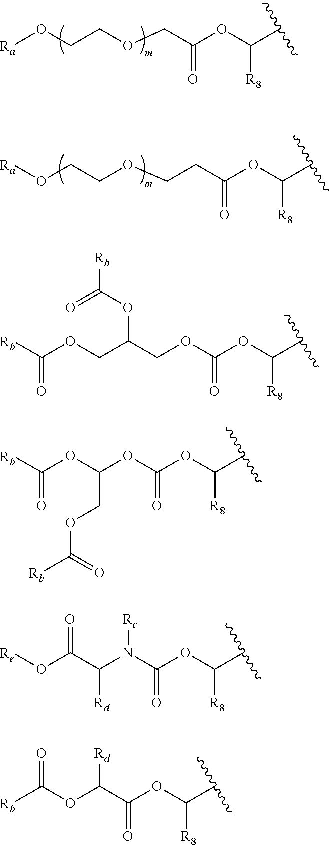

wherein m is 1 to about 1000, preferably 1 to about 100; Rₐ, R_{b} and Rₑ are each independently C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, C₃-C₁₂-cycloalkenyl, substituted C₃-C₁₂-cycloalkenyl, aryl or substituted aryl; R_{c} is H or substituted or unsubstituted C₁-C₆-alkyl; R_{d} is H, substituted or unsubstituted C₁-C₆-alkyl, substituted or unsubstituted aryl-C₁-C₆-alkyl or substituted or unsubstituted heteroaryl-C₁-C₆-alkyl; and R₈ is as defined above and is preferably hydrogen. Preferably Rₐ, R_{b} and Rₑ are each C₁-C₂₄-alkyl. Preferably R_{d} is the side chain of one of the twenty naturally occurring amino acids, more preferably a neutral or hydrophobic side chain, such as hydrogen, methyl, isopropyl, isobutyl, benzyl, indolylmethyl, and sec-butyl. R_{c} and R_{d} can also, together with the carbon and nitrogen atoms to which they are attached, form a heterocycloalkyl group, preferably a pyrrolidine group.

In one embodiment, the compounds of the invention are represented by Formula V,

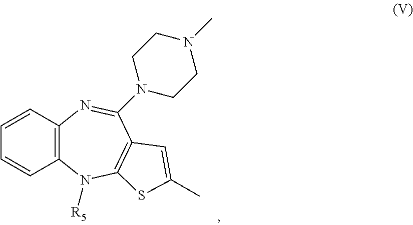

or a pharmaceutically acceptable salt thereof, wherein R₅ is as previously defined.

In one embodiment, the compound of the invention is represented by Formula VI,

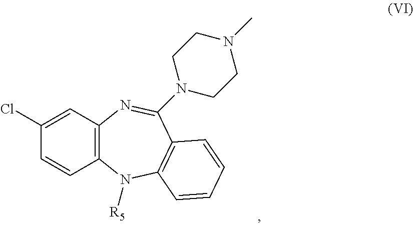

or a pharmaceutically acceptable salt thereof, wherein R₅ is as previously defined.

In another embodiment, the invention provides a compound of Formula VII, VIII, IX or X:

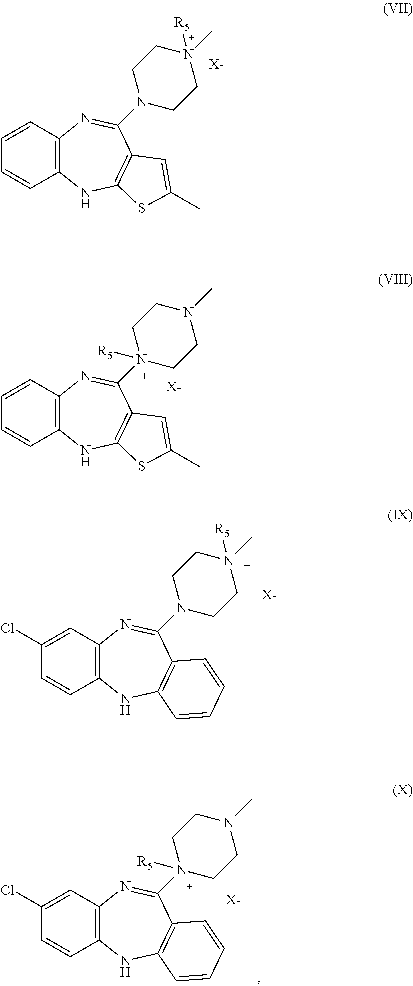

wherein R₅ and X⁻ are as previously defined.

The compounds of the invention are prodrugs of parent drug compounds, such as olanzapine, clozapine and compounds represented by formulas XI, XII and XIII:

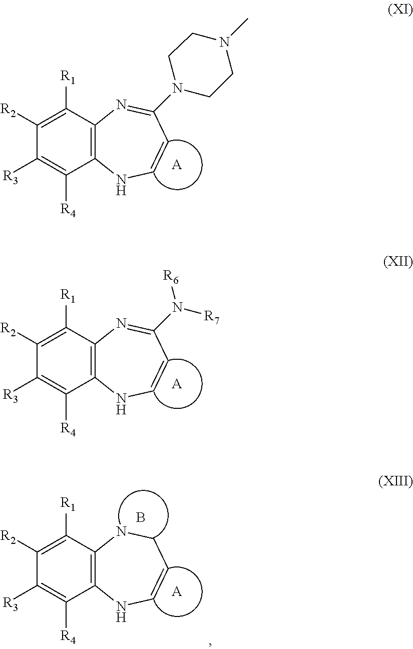

where Rings A and B, R₁-R₄, R₆ and R₇ have the meanings given above.

In one embodiment, variable R₅ in any of Formulas I-X is selected from the group set forth in Table 1 below.

TABLE 1

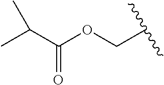

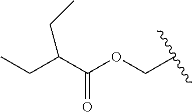

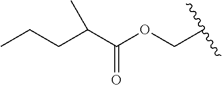

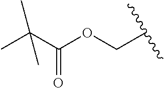

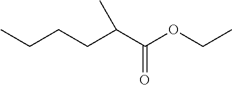

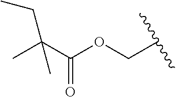

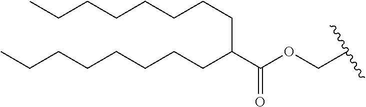

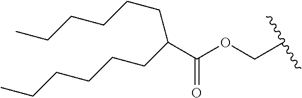

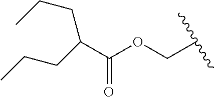

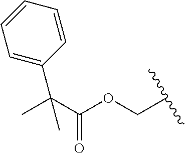

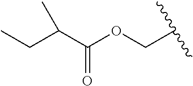

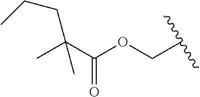

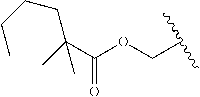

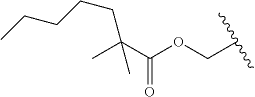

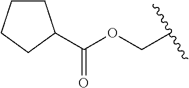

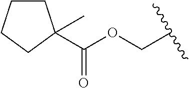

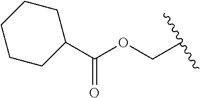

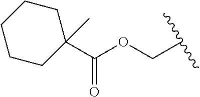

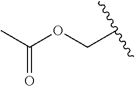

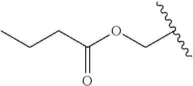

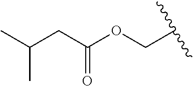

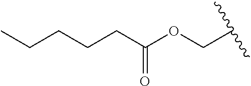

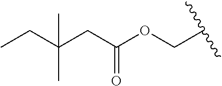

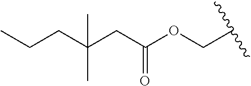

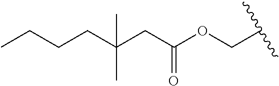

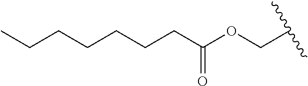

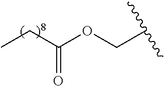

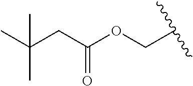

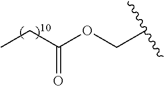

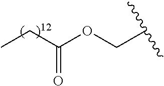

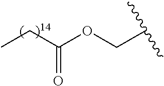

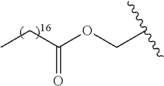

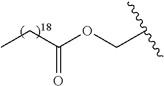

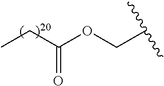

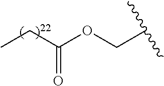

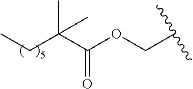

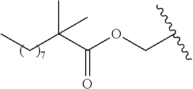

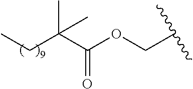

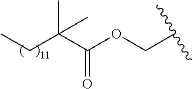

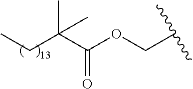

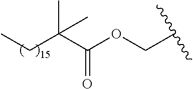

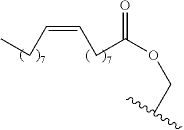

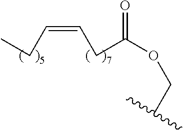

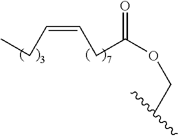

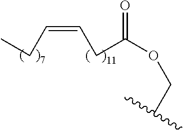

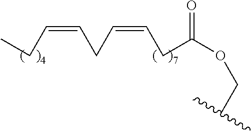

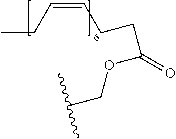

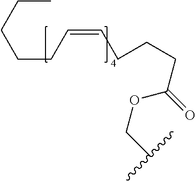

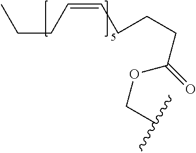

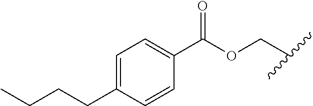

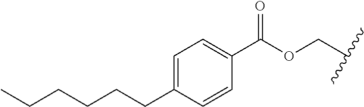

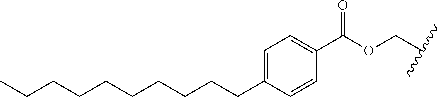

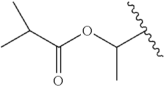

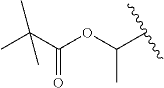

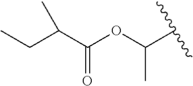

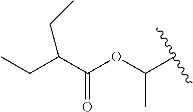

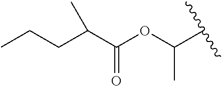

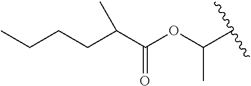

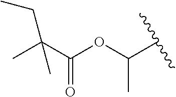

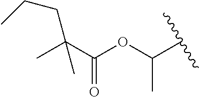

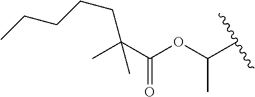

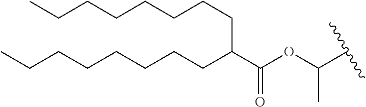

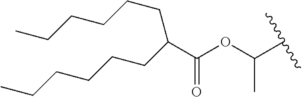

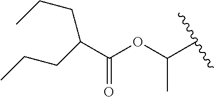

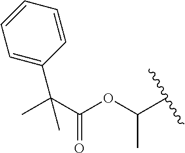

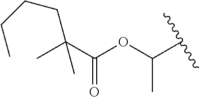

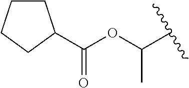

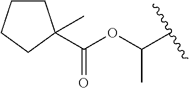

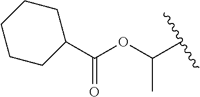

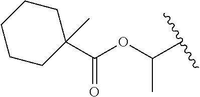

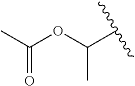

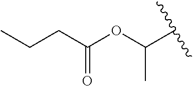

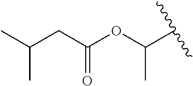

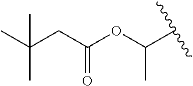

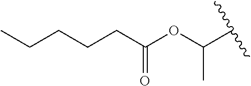

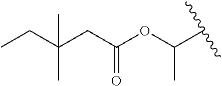

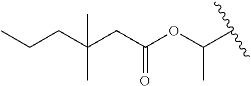

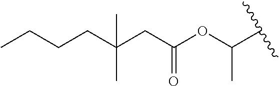

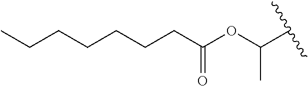

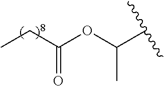

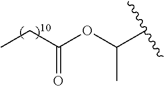

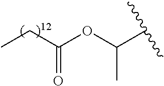

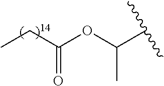

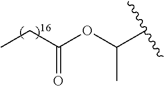

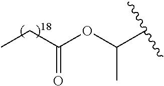

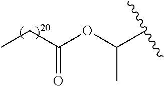

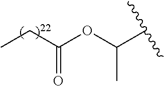

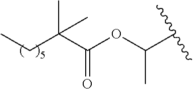

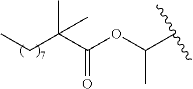

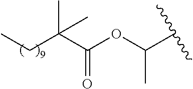

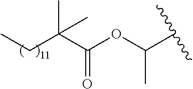

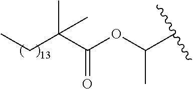

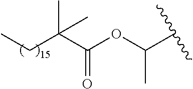

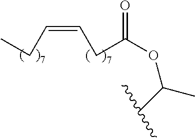

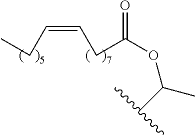

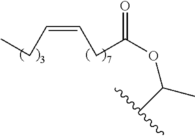

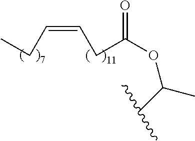

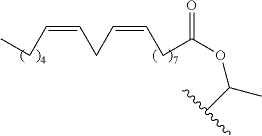

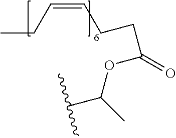

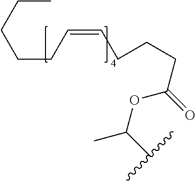

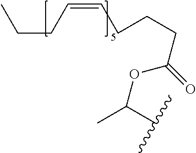

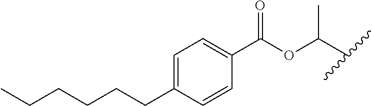

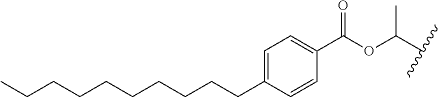

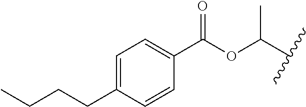

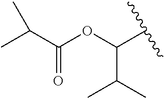

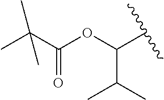

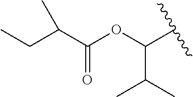

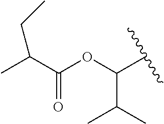

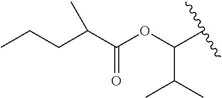

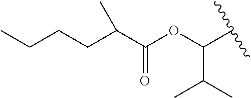

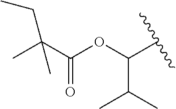

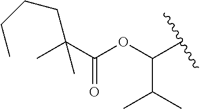

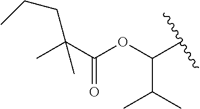

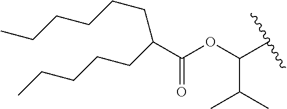

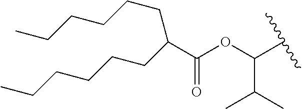

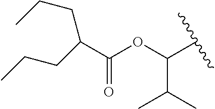

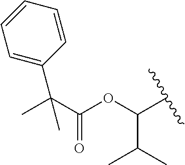

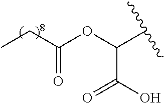

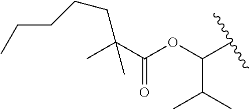

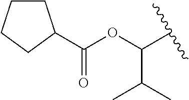

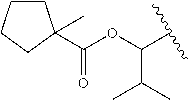

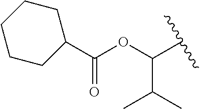

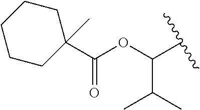

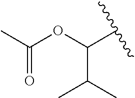

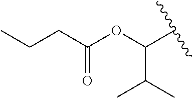

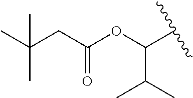

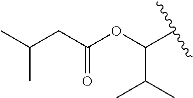

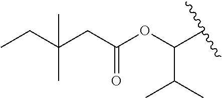

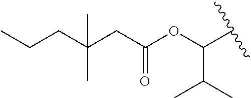

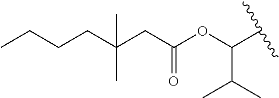

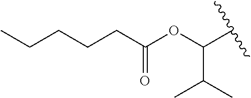

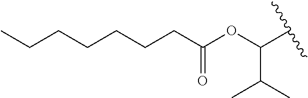

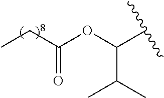

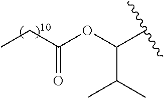

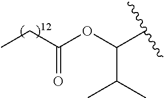

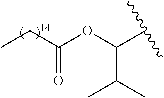

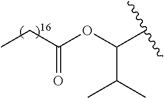

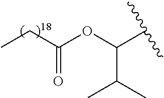

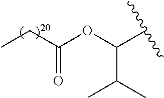

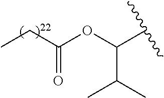

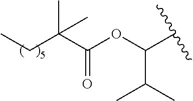

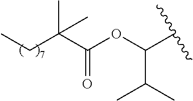

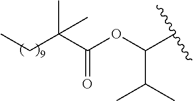

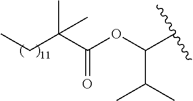

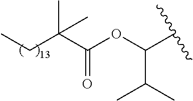

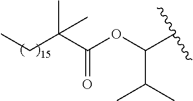

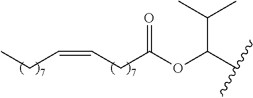

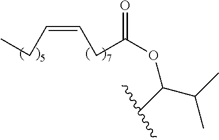

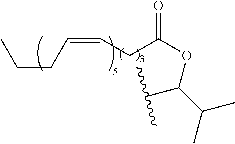

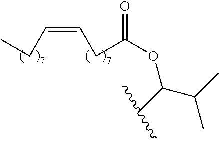

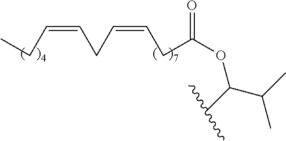

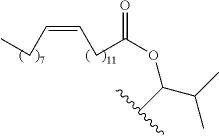

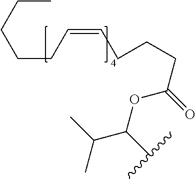

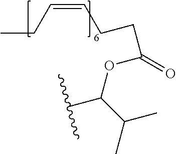

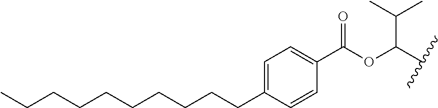

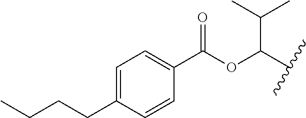

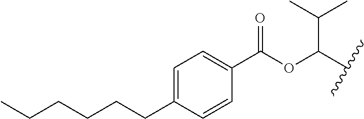

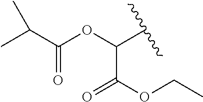

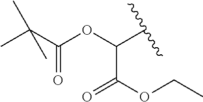

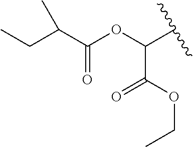

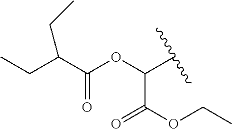

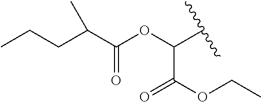

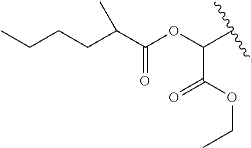

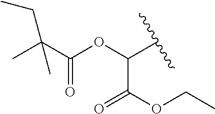

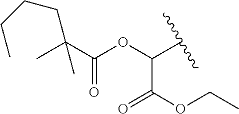

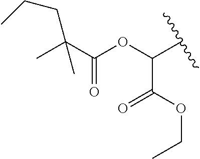

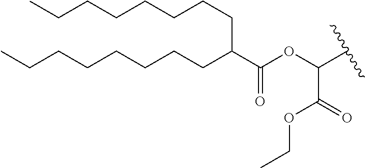

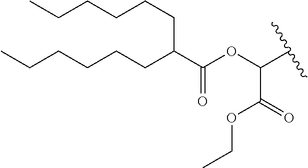

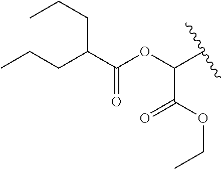

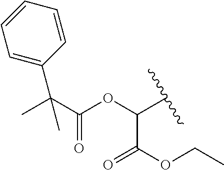

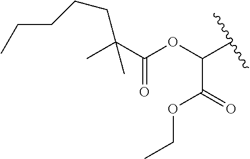

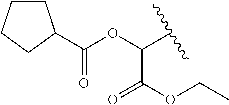

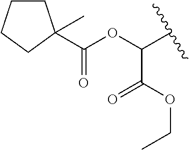

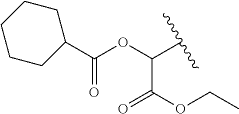

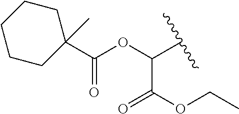

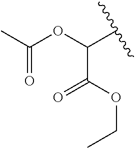

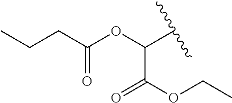

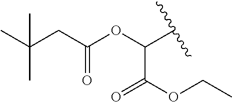

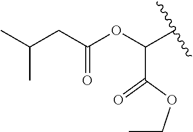

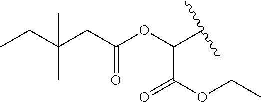

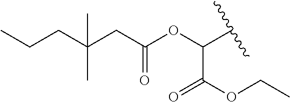

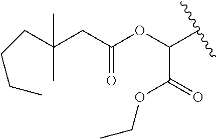

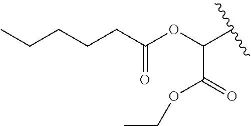

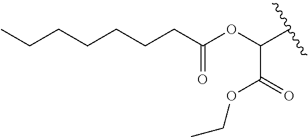

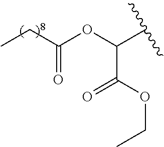

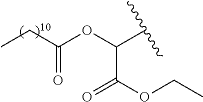

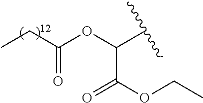

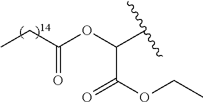

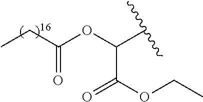

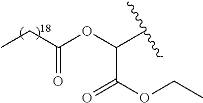

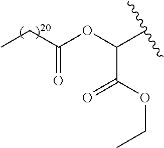

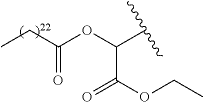

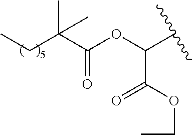

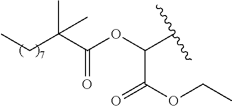

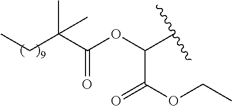

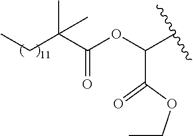

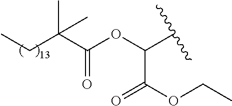

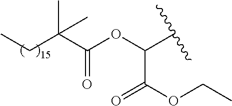

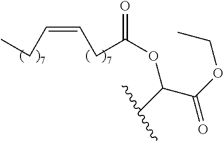

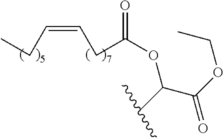

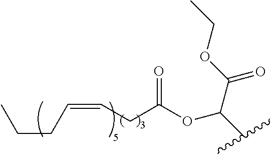

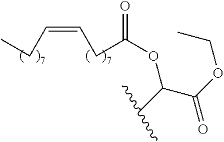

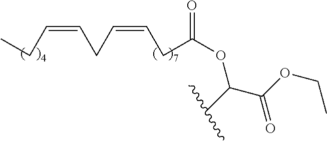

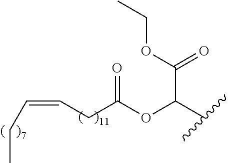

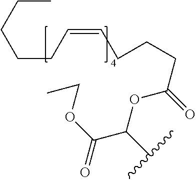

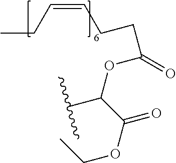

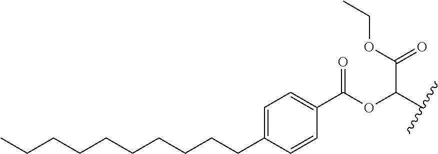

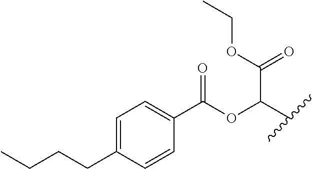

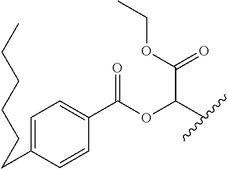

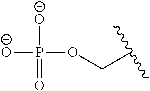

MY 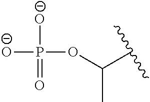
MY 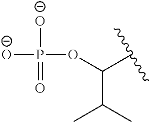
MY 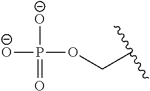
Ca²⁺ 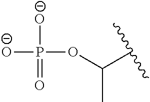
Ca²⁺ 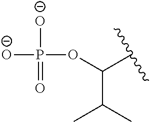
Ca²⁺

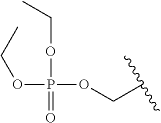

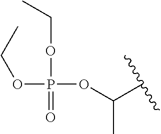

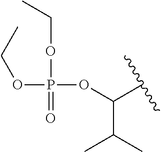

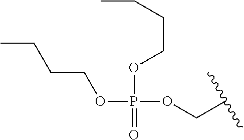

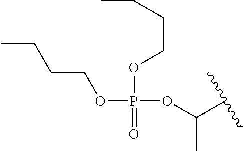

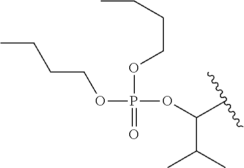

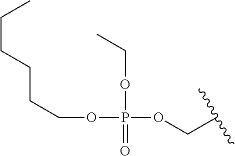

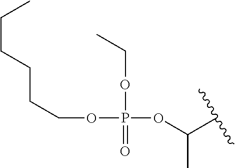

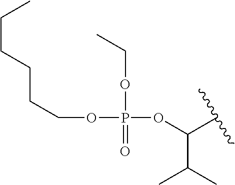

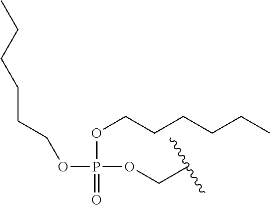

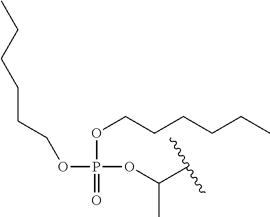

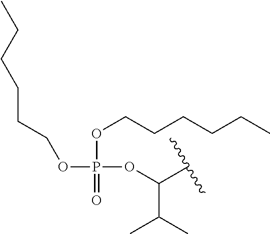

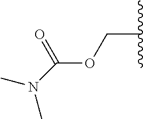

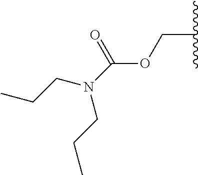

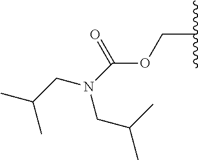

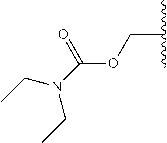

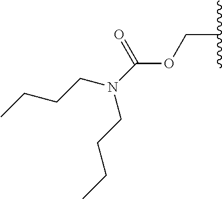

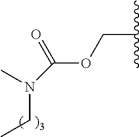

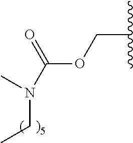

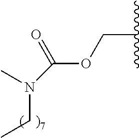

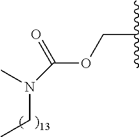

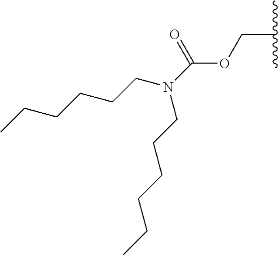

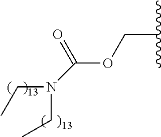

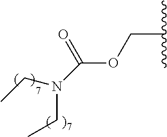

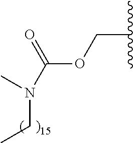

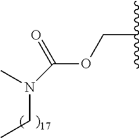

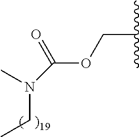

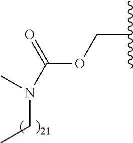

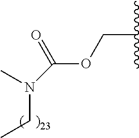

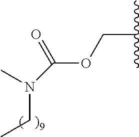

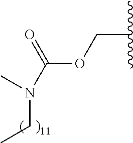

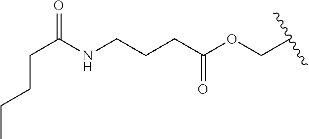

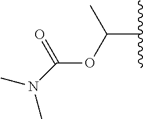

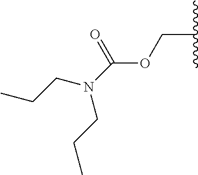

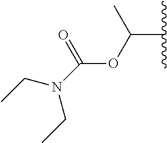

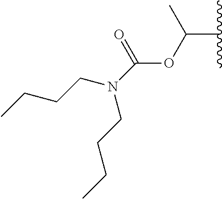

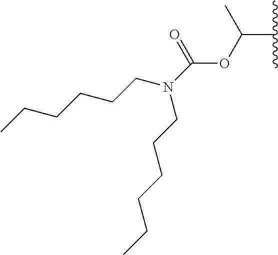

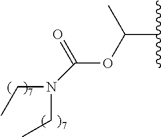

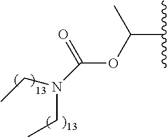

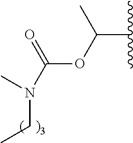

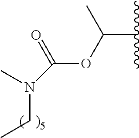

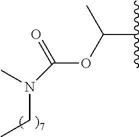

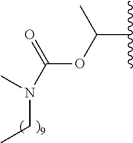

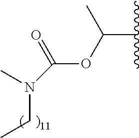

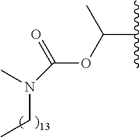

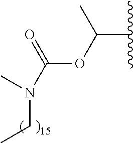

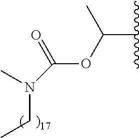

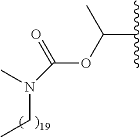

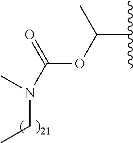

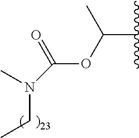

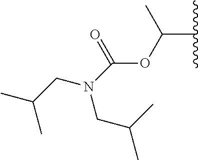

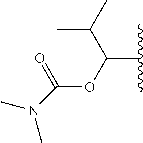

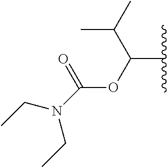

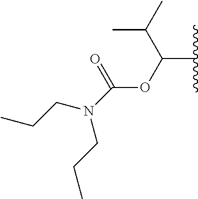

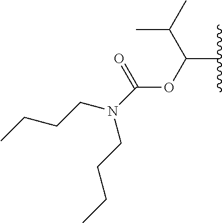

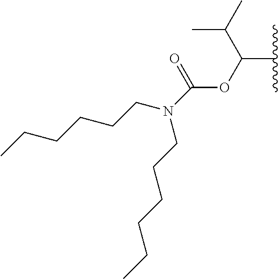

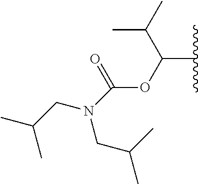

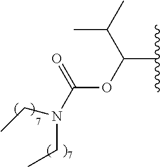

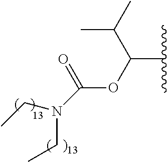

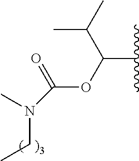

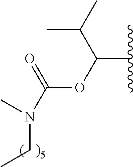

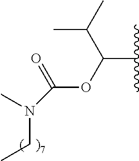

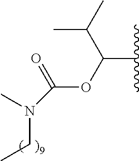

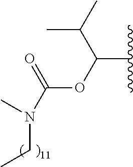

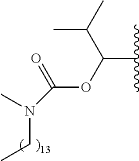

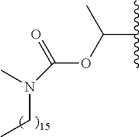

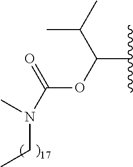

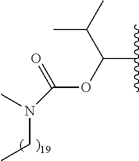

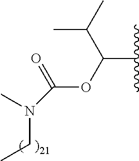

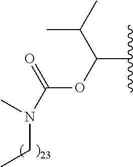

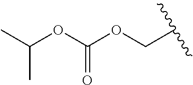

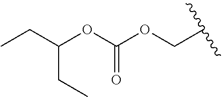

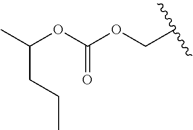

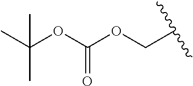

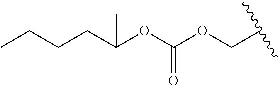

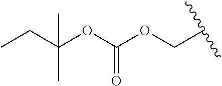

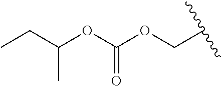

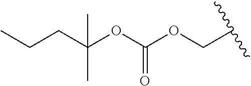

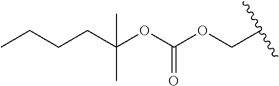

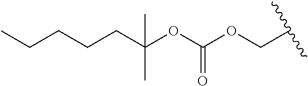

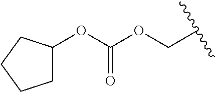

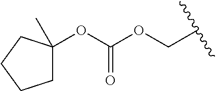

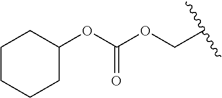

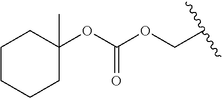

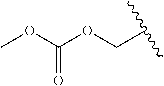

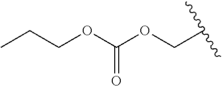

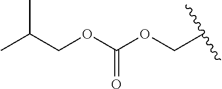

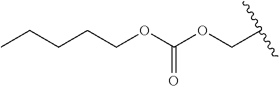

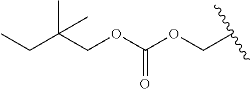

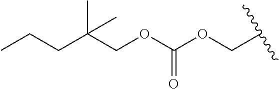

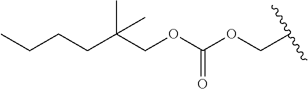

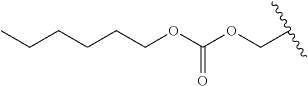

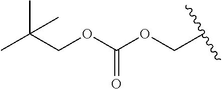

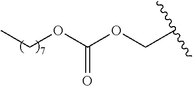

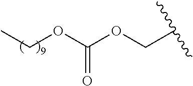

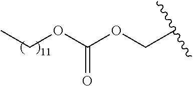

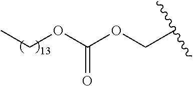

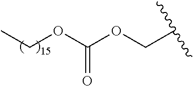

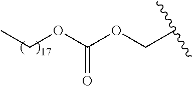

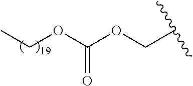

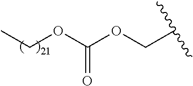

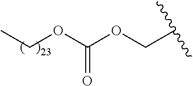

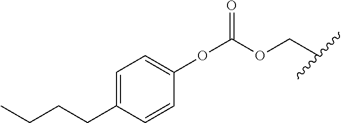

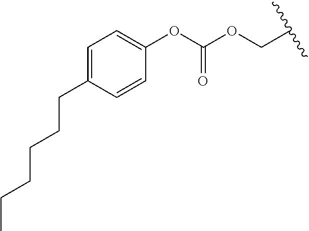

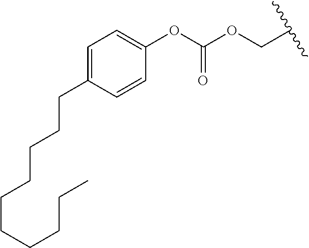

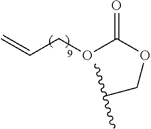

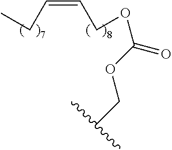

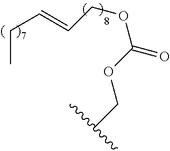

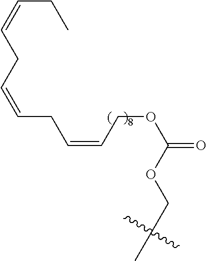

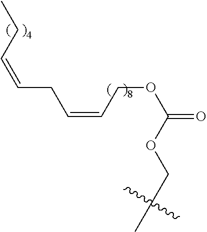

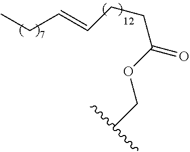

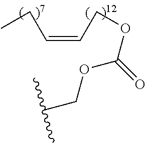

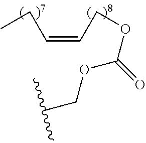

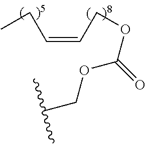

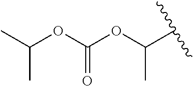

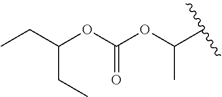

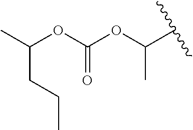

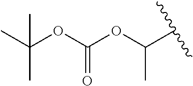

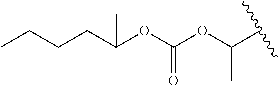

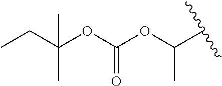

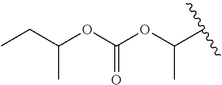

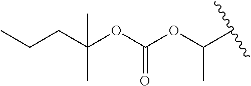

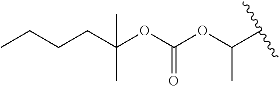

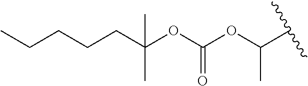

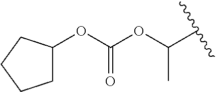

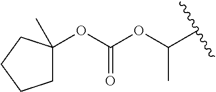

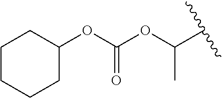

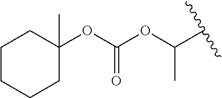

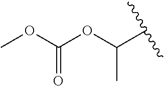

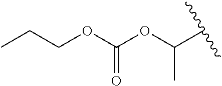

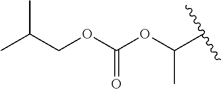

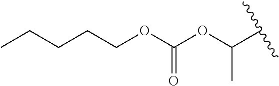

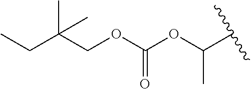

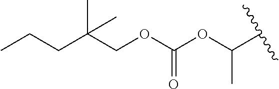

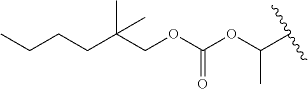

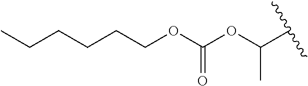

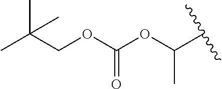

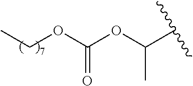

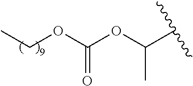

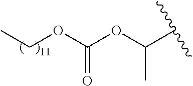

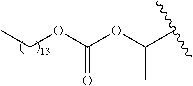

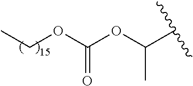

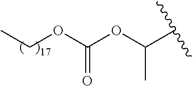

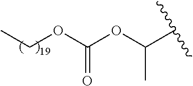

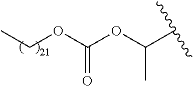

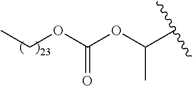

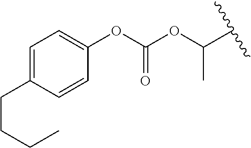

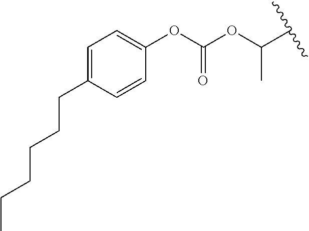

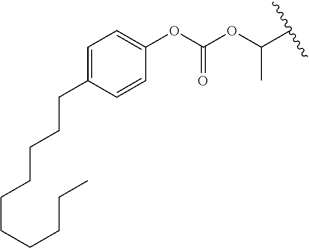

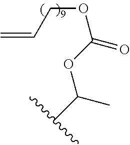

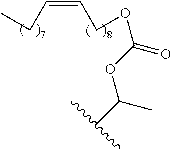

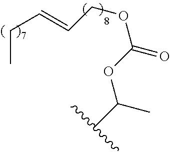

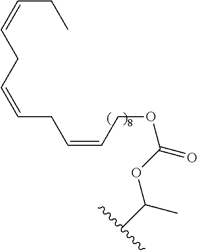

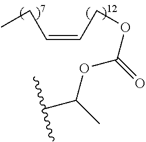

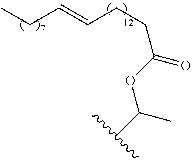

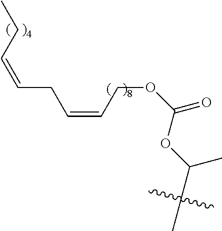

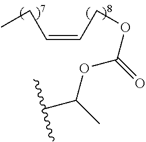

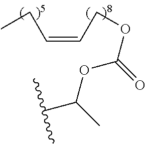

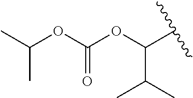

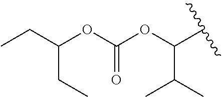

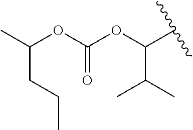

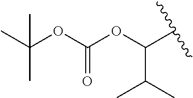

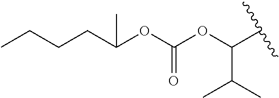

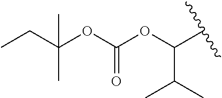

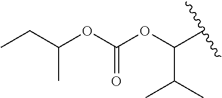

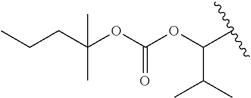

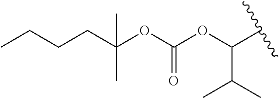

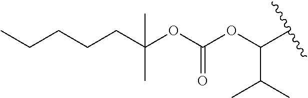

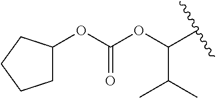

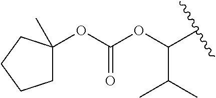

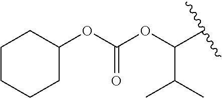

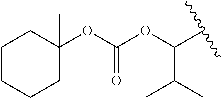

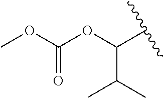

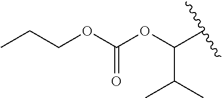

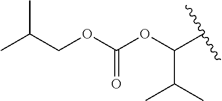

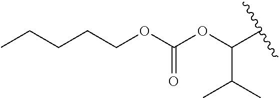

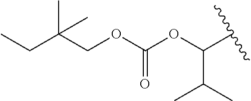

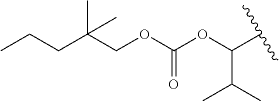

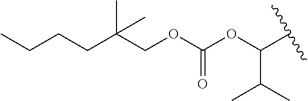

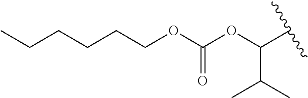

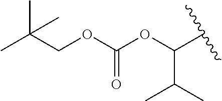

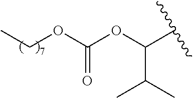

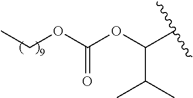

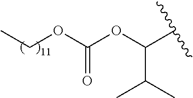

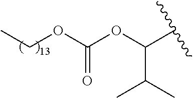

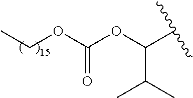

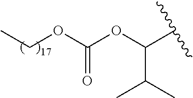

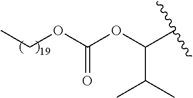

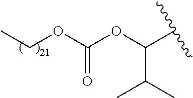

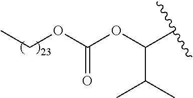

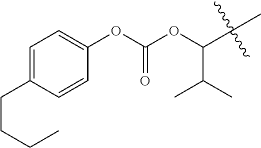

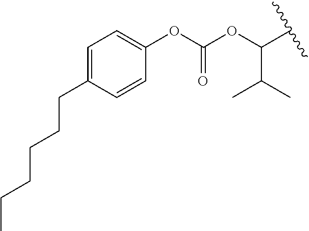

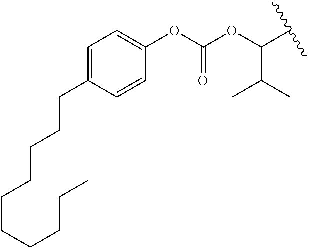

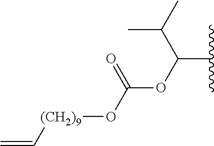

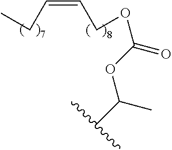

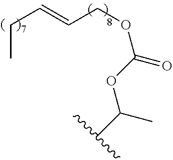

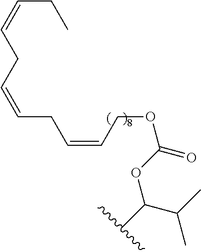

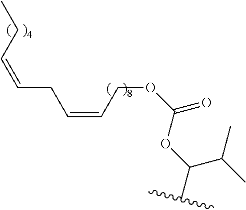

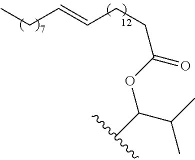

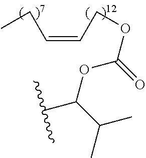

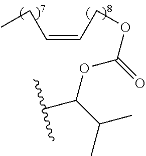

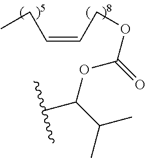

In preferred embodiments, variable R₅ in any of Formulas I-X is selected from the group set forth in Tables 2-5 below.

TABLE 2

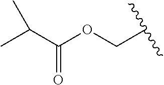

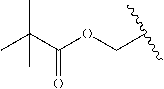

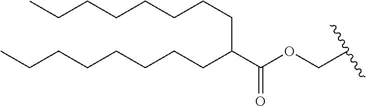

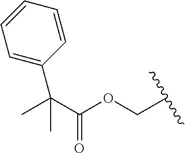

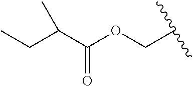

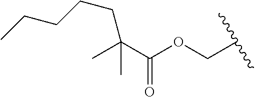

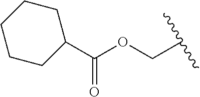

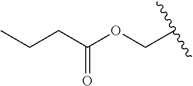

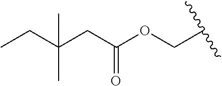

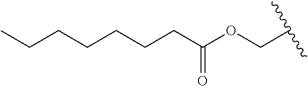

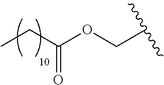

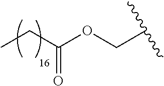

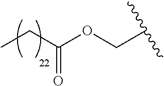

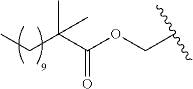

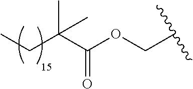

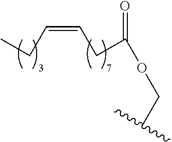

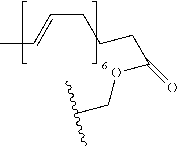

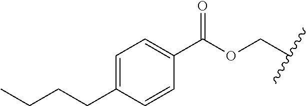

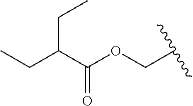

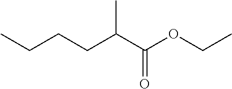

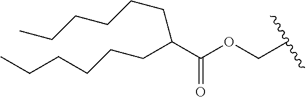

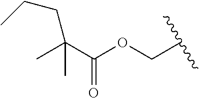

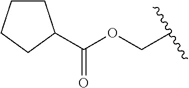

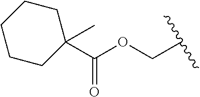

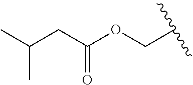

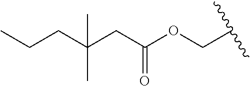

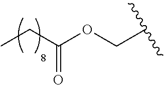

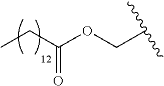

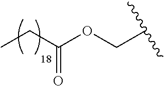

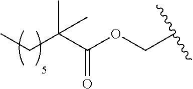

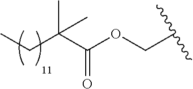

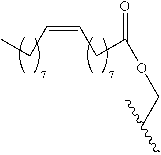

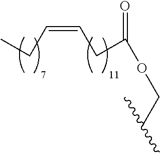

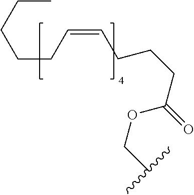

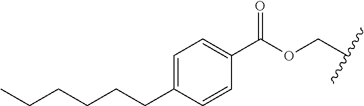

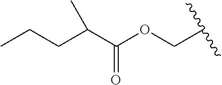

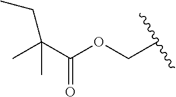

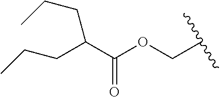

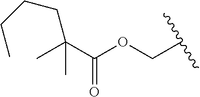

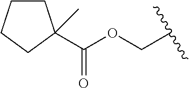

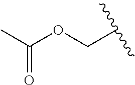

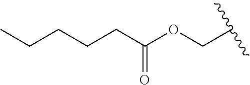

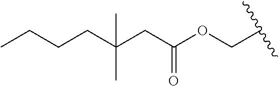

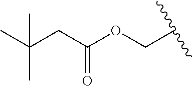

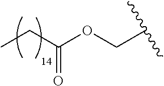

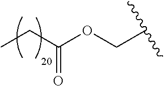

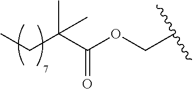

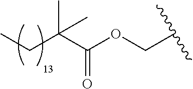

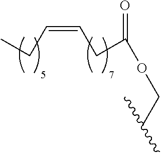

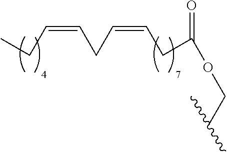

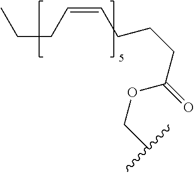

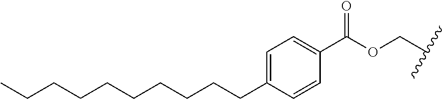

TABLE 3

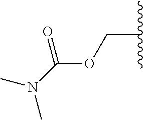

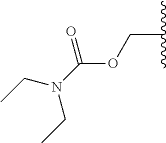

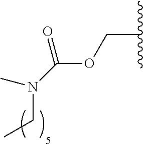

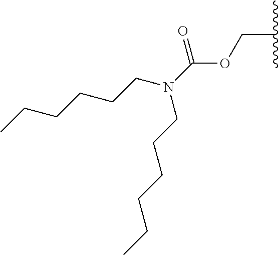

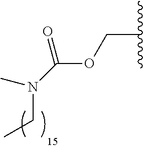

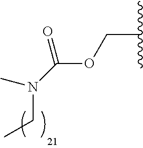

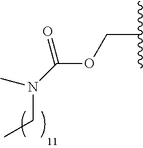

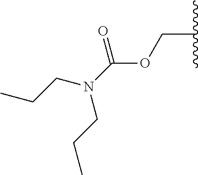

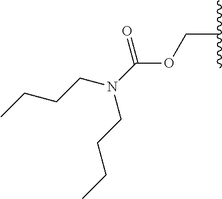

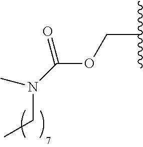

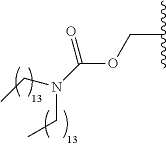

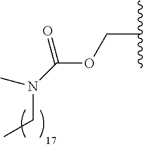

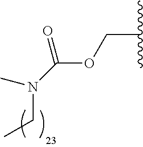

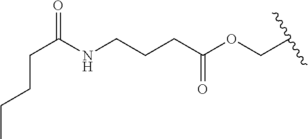

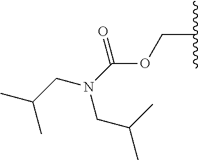

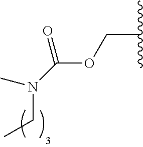

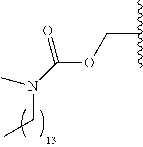

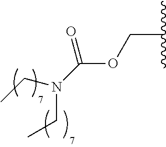

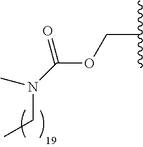

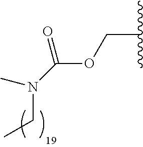

TABLE 4

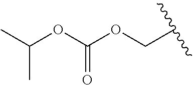

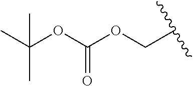

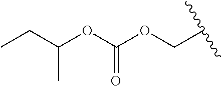

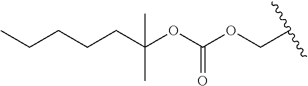

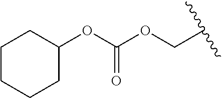

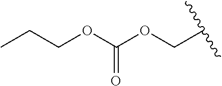

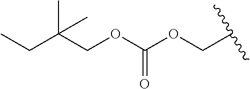

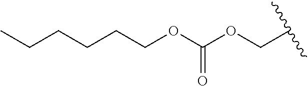

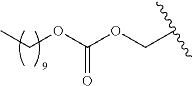

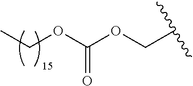

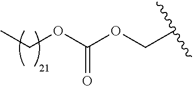

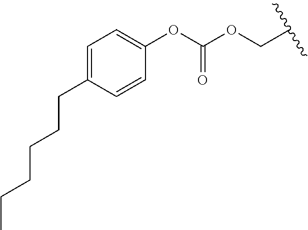

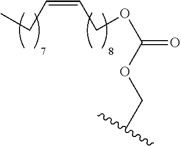

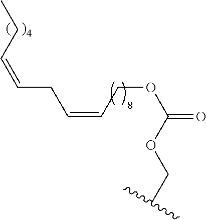

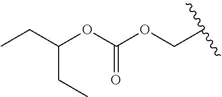

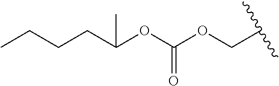

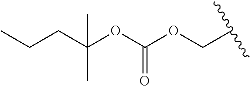

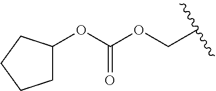

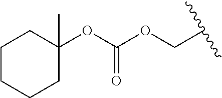

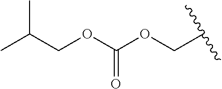

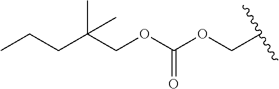

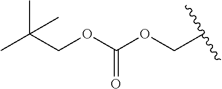

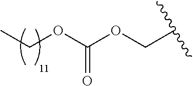

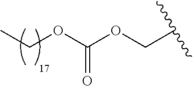

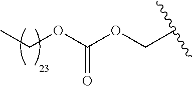

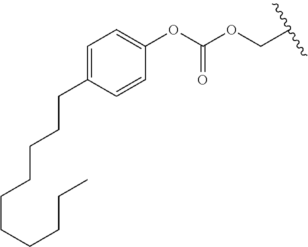

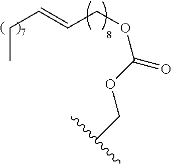

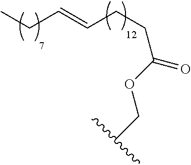

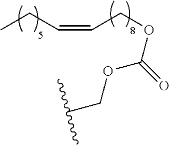

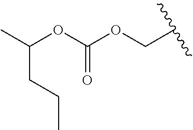

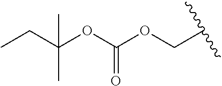

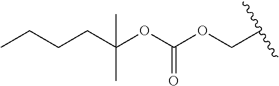

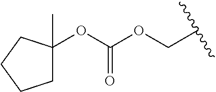

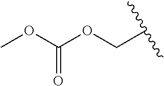

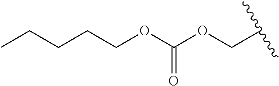

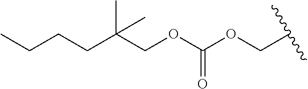

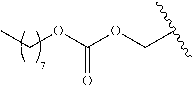

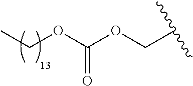

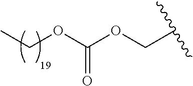

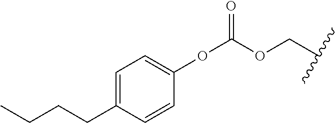

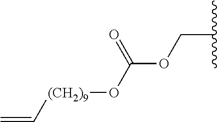

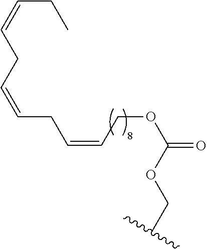

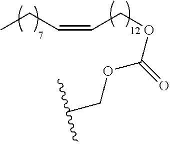

TABLE 5

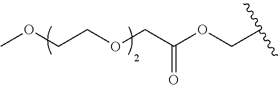

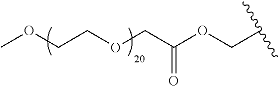

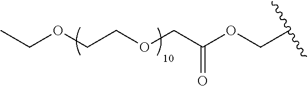

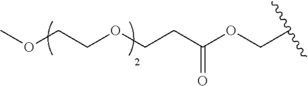

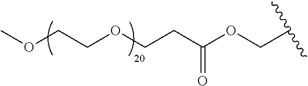

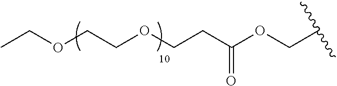

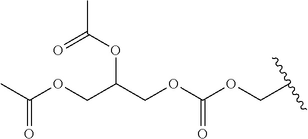

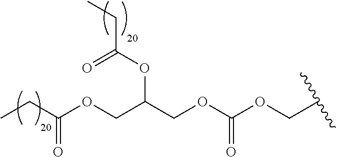

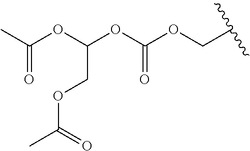

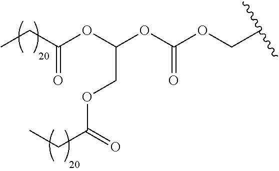

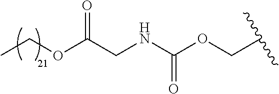

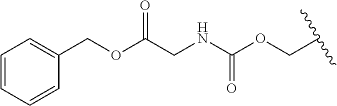

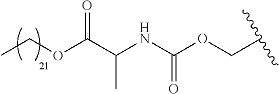

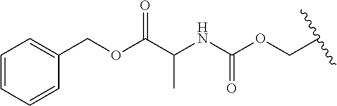

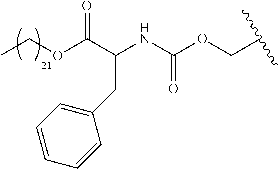

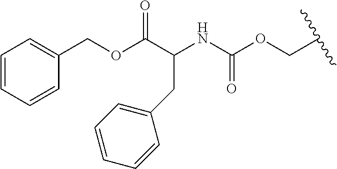

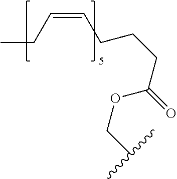

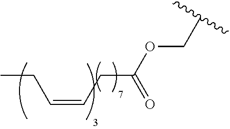

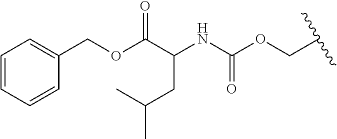

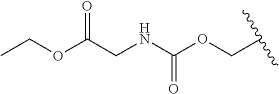

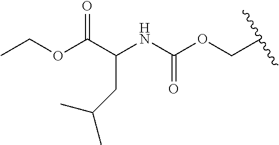

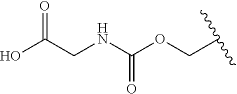

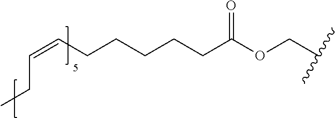

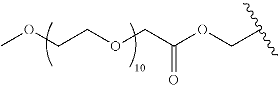

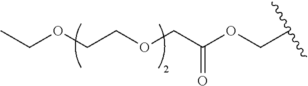

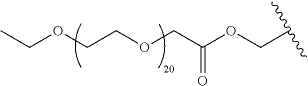

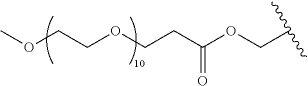

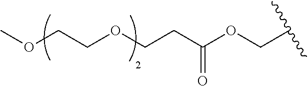

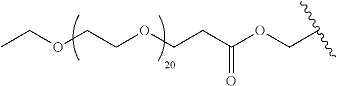

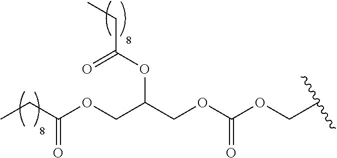

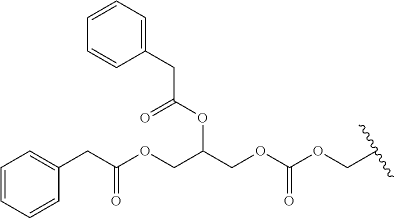

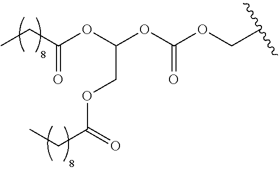

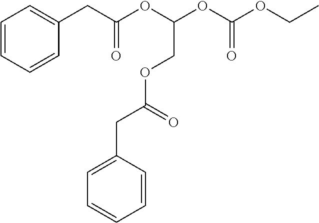

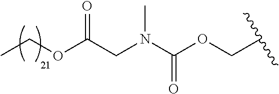

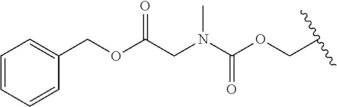

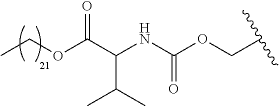

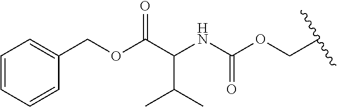

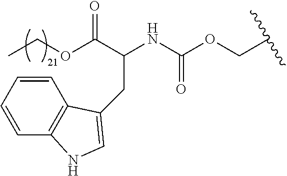

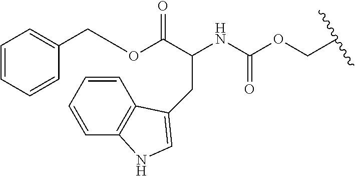

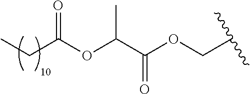

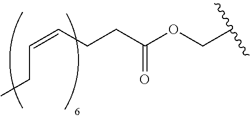

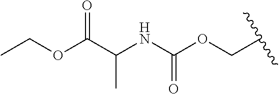

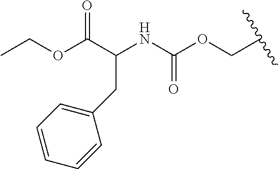

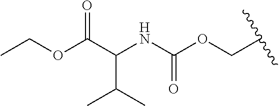

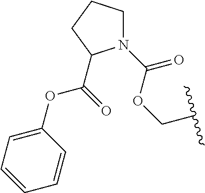

In a preferred embodiment, a compound of the Formulas III, IV, V and VI is less soluble under physiological conditions than the parent drug. In one embodiment, a compound of Formulas III, IV, V and VI of the invention has a solubility of less than about 0.1 mg/mL, 0.05 mg/mL, 0.01 mg/mL, 0.005 mg/mL, 0.001 mg/mL, 0.0005 mg/mL, 0.0001 mg/mL, 0.00005 mg/mL or 0.00001 mg/mL at room temperature in aqueous phosphate buffer at pH 7.4.

In another embodiment, the compounds of the invention that are quaternary amine containing salts such as compounds of Formulas I, II, VII, VIII, IX and X are less soluble at a reference pH than the parent drug from which they were derived. As used herein the term “reference pH” refers to the pH at which the aqueous solubility of a prodrug of the invention is compared to the aqueous solubility of the parent drug (not in prodrug form). Generally the reference pH is the pH at which the parent drug is essentially fully protonated (i.e., at least about 99% protonated). Typically, the reference pH is about 5 and is preferably in the range of 4-7. Preferably, the aqueous solubility of a quaternary amine-containing prodrug compound of the invention at the reference pH is at least an order of magnitude lower than the aqueous solubility of the parent drug.

In one embodiment, a quaternary amine-containing prodrug of the invention (i.e. a compound of Formula I, II, VII, VIII, IX or X) has a solubility in an aqueous phosphate buffer at room temperature of less than about 0.1 mg/mL, 0.05 mg/mL, 0.01 mg/mL, 0.005 mg/mL, 0.001 mg/mL, 0.0005 mg/mL, 0.0001 mg/mL, 0.00005 mg/mL or 0.00001 mg/mL at a pH of about 6.

Other embodiments of the invention exploit the pH-independent aqueous solubility of the quaternary ammonium-containing prodrugs of the invention. A key advantage of the prodrugs of formulas I, II and VII-X over their parent, tertiary amine-containing drugs, is that the prodrug solubility remains essentially unchanged between pH 3 and 8, while the solubility of the tertiary amine parent drugs commonly increases by more than 100-fold over this pH range. The extent of solubilization accompanying pH reduction across this range depends on drug base solubility, pKa of the conjugate acid and counterions in the medium forming the ammonium salt. It is known in the art that biological tissues can become inflamed in response to injections, and that the pH of the inflamed tissue typically decreases from 7.1-7.4 down to pH 6.4 (See: A Dominant Role of Acid pH in Inflammatory Excitation and Sensitization of Nociceptors in Rat Skin, in vitro. Steen, K. H.; Steen, A. E.; Reeh, P. W. The Journal of Neuroscience, (1995), 15: pp. 3982-3989). Transient pH in inflamed tissue can sometimes be as low as pH 4.7. Exercise alone can bring about a pH drop of about 0.5 units for up to 30 minutes (see: Continuous intramuscular pH measurement during the recovery from brief, maximal exercise in man. Allsop P; Cheetham M; Brooks S; Hall G M; Williams C. European journal of applied physiology and occupational physiology (1990), 59(6), pp. 465-70). It has also been demonstrated that release of drug from sustained release formulations can become rapid with reduced pH from subcutaneous space (see: Effect and interaction of pH and lidocaine on epinephrine absorption. Ueda, Wasa; Hirakawa, Masahisa; Mori, Koreaki, Anesthesiology (1988), 68(3), pp. 459-62), leading to a “burst” or “dumping” effect if the local pH drops at the injection site. It is hypothesized that this apparent failure of the formulations is caused by the high solubility of the drug at the lower pH. Therefore, even if the solubility of a given prodrug is similar to that of the corresponding parent tertiary amine at pH 7, the pH-independent solubility profile of the prodrug means that solubility is controlled by the formulation without concern over dose-dumping in response to injection site irritation or, more generally, by pH fluctuations caused by patient activities, therapeutic interventions or illness.

Sustained release drug formulations often contain higher amounts of drugs than immediate release formulations. Functionality and safety of a sustained release formulation are based on a reliable and controlled rate of drug release from the formulation over an extended period of time after administration. The drug release profile of a formulation often depends on the chemical environment of the sustained release formulation, for example, on pH, ionic strength and presence of solvents such as ethanol.

The relatively high amount of drug that is present in a sustained release formulation can, in some instances, harm a patient if the formulation releases the drug at a rate that is faster than the intended controlled release rate. If the formulation releases the drug at a rate that is slower than the intended controlled release rate, the therapeutic efficacy of the drug can be reduced.

In most cases, partial or total failure of a sustained release formulation results in a rapid release of the drug into the bloodstream. This rapid release is generally faster than the intended sustained release of the drug from the formulation, and is sometimes referred to as “dose dumping.”

Dose dumping can create severe consequences for a patient, including permanent harm and even death. Examples of drugs that can be fatal if the therapeutically beneficial dose is exceeded, e.g., by dose dumping, include pain medications such as opioids, as well as other agents active in the central nervous system. In those situations where dose dumping may not be fatal, dose dumping may at least be responsible for the side effect of sedation or coma in the patient.

The present invention solves the problem of dose dumping in a sustained release formulation, including, for example, sedation or coma, by providing prodrugs, including quaternary-amine containing prodrugs, that maintain their reduced solubility and sustained release action in a manner which is independent of the pH of the environment in which the prodrug is administered. The pH-independent solubility of the quaternary amine-containing prodrugs of the invention is an important feature for drugs that are administered both orally and by injection. During oral administration, the prodrugs of the invention are exposed to a variety of pHs including very low pHs in the stomach (e.g. pH 2.0) and then increased pH when crossing the intestinal walls into the bloodstream. During injection it has been observed that the pH at the injection site may also be lowered (e.g. below pH 6.0). Poster #242, Controlled Release Society (CRS) Annual Meeting, Copenhagen, Denmark (July 2009); and Steen, K. H.; Steen, A. E.; Reeh, P. W. The Journal of Neuroscience, (1995), 15: pp. 3982-3989. The pH of an injection site may be lowered for a short amount of time (1-2 hours), but the perturbation may be sufficient to dissolve a basic drug having pH-dependent solubility. In accordance with the invention, the reduced solubility of the prodrugs of the invention remains independent of any change in pH. In one preferred embodiment the reduced solubility of the prodrugs of the invention remains independent over a pH range of pH 4 to pH 9. More preferably the reduced solubility of the prodrugs of the invention remains independent over a pH range of pH 3 to pH 10. Most preferably, the reduced solubility of the prodrugs of the invention remains independent over a pH range of 1.0 to 11.

In addition, it is known that the stability of carboxyl ester linkages, such as those contemplated in certain prodrugs of the invention, is dependent on pH with optimum stability occurring at around pH 4-5. If injection site pH fluctuates to a value lower than neutral pH of 7.4, then the stability of the prodrug is increased relative to neutral pH. This stability increase further reduces the risk of early release of active drug from the compound, and thus avoids dose dumping by way of accelerated chemical cleavage of the prodrug.

Therefore the present invention further provides methods of pH-independent sustained release delivery of a parent drug to a subject, comprising administering to the subject a prodrug of the invention to a patient, such as a prodrug of any of Formulas I-X, preferably, Formulas I, II, VII, VIII, IX or X, to the patient.

The invention also provides methods for reducing sedation or coma in a patient as compared to the parent drug comprising administering to a patient a therapeutically effective amount of a compound of Formulas I-X to the patient.

In a preferred embodiment, a compound of the invention provides sustained delivery of the parent drug over hours, days, weeks or months when administered, for example, orally or parenterally, to a subject. For example, the compounds can provide sustained delivery of the parent drug for up to 7, 15, 30, 60, 75 or 90 days or longer. Without being bound by theory, it is believed that the compounds of the invention form an insoluble depot upon parenteral administration, for example subcutaneous, intramuscular or intraperitoneal injection.

The term “labile” as used herein refers to the capacity of the prodrug of the invention to undergo enzymatic and/or chemical cleavage in vivo thereby forming the parent diaryldiazepine parent drug. As used herein the term “prodrug” means a compounds as disclosed herein which is a labile derivative compound of a diaryldiazepine parent drug which when administered to a patient in vivo becomes cleaved by chemical and/or enzymatic hydrolysis thereby forming the parent drug such that a sufficient amount of the compound intended to be delivered to the patient is available for its intended therapeutic use in a sustained release manner. As used herein the term “diaryldiazepine parent drug” means any diaryldiazepine compound that is used in the prevention, diagnosis, treatment, or cure of disease, for the relief of pain or to control or improve the underlying cause or symptoms associated with any physiological or pathological disorder in humans or animals.

The compounds of the invention can be prepared as acid addition salts. Preferably, the acid is a pharmaceutically acceptable acid. Such acids are described in Stahl, P. H. and Wermuth, C. G. (eds.), Handbook of Pharmaceutical Salts: Properties, Selection and Use, Wiley VCH (2008). Pharmaceutically acceptable acids include acetic acid, dichloroacetic acid, adipic acid, alginic acid, L-ascorbic acid, L-aspartic acid, benzenesulfonic acid, 4-acetamidobenzoic acid, benzoic acid, p-bromophenylsulfonic acid; (+)-camphoric acid, (+)-camphor-10-sulfonic acid, capric acid, caproic acid, caprylic acid, carbonic acid, cinnamic acid, cyclamic acid, dodecylsulfuric acid, ethane-1,2-disulfonic acid, ethanesulfonic acid, 2-hydroxyethanesulfonic acid, sulfuric acid, boric acid, citric acid, formic acid, fumaric acid, galactaric acid, gentisic acid, D-glucoheptonic acid, D-gluconic acid, D-glucuronic acid, glutamic acid, glutaric acid, 2-oxoglutaric acid, glycerophosphoric acid, glycolic acid, hippuric acid, hydrochloric acid, hydrobromic acid, hydroiodic acid, isobutyric acid, DL-lactic acid, lactobionic acid, lauric acid, maleic acid, (−)-L-malic acid, malonic acid, DL-mandelic acid, methanesulfonic acid, naphthalene-1,5-disulfonic acid, naphthalene-2-sulfonic acid, 1-hydroxy-2-naphthoic acid, nicotinic acid, nitric acid, oleic acid, orotic acid, oxalic acid, palmitic acid, pamoic acid, phosphoric acid, propionic acid, (−)-L-pyroglutamic acid, salicyclic acid, 4-aminosalicyclic acid, sebacic acid, stearic acid, succininc acid, (+)-L-tartaric acid, thiocyanic acid, p-toluenesulfonic acid, and undecylenic acid.

The term “pharmaceutically acceptable anion” as used herein, refers to the conjugate base of a pharmaceutically acceptable acid. Such anions include the conjugate base of any the acids set forth above. Preferred pharmaceutically acceptable anions include acetate, bromide, camsylate, chloride, formate, fumarate, maleate, mesylate, nitrate, oxalate, phosphate, sulfate, tartrate, thiocyanate, iodide and tosylate.

### Methods of Treating a Psychiatric or Neurological Disorder

In one embodiment, the present invention provides a method of treating a neurological or psychiatric disorder in a patient in need thereof. The method comprises administering to the subject a therapeutically effective amount of a compound of the invention or a pharmaceutically acceptable salt thereof.

The term “neurological or psychiatric disorder”, as this term is used herein, is a disease or disorder of the central nervous system that is manifested in mood and/or behavioral abnormalities. Examples of neurological or psychiatric disorders include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD)), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), attention deficit/hyperactivity disorder, and conduct disorder.

The term “treatment” refers to any process, action, application, therapy, or the like, wherein a mammal, including a human being, is subject to medical aid with the object of improving the mammal's condition, directly or indirectly.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha- (α), beta- (β) and gamma- (γ) cyclodextrins; starches such as corn starch and potato starch; cellulose and its derivatives such as sodium carboxymethylcellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir. In a preferred embodiment, administration is parenteral administration by injection.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, acetamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Intralipid® is an intravenous fat emulsion containing 10-30% soybean oil, 1-10% egg yolk phospholipids, 1-10% glycerin and water. Liposyn® is also an intravenous fat emulsion containing 2-15% safflower oil, 2-15% soybean oil, 0.5-5% egg phosphatides 1-10% glycerin and water. Omegaven® is an emulsion for infusion containing about 5-25% fish oil, 0.5-10% egg phosphatides, 1-10% glycerin and water. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Additional sustained release in accordance with the invention may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

In one preferred embodiment, the formulation provides a sustained release delivery system that is capable of minimizing the exposure of the prodrug to water. This can be accomplished by formulating the prodrug with a sustained release delivery system that is a polymeric matrix capable of minimizing the diffusion of water into the matrix. Suitable polymers comprising the matrix include poly(lactide) (PLA) polymers and the lactide/(glycolide) (PLGA) copolymers as described earlier.

Alternatively, the sustained release delivery system may comprise poly-anionic molecules or resins that are suitable for injection or oral delivery. Suitable polyanionic molecules include cyclodextrins and polysulfonates formulated to form a poorly soluble mass that minimizes exposure of the prodrug to water and from which the prodrug slowly leaves. Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference). A discussion of pulmonary delivery of antibiotics is also found in U.S. Pat. No. 6,014,969, incorporated herein by reference.

In preferred embodiments, the compounds of the invention, or pharmaceutical compositions comprising one or more compounds of the invention, are administered parenterally, for example, by intramuscular, subcutaneous or intraperitoneal injection. Without being bound by theory, it is believed that upon injection, compounds of the invention form an insoluble or sparingly soluble depot from which prodrug molecules are released over time.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, the possibility of co-usage with other agents and the duration of release of the parent drug. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 5 mg to about 1000 mg of the compound(s) of this invention per day in single or multiple doses.

The compounds of the invention can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about once per day to about once per week, once every two weeks, once per month or less frequently. Such administration can be used as a chronic or acute therapy. The amount of active ingredient that may be combined with pharmaceutically acceptable excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Preferred compounds of the invention exhibit sustained activity following dosing compared to dosing with the parent drug. For example, when administered by the same route in the same amount (as measured by equivalents of parent drug), the compounds of the invention provide sustained therapeutic serum levels of parent drug for a significantly longer time than the parent drug. Such administration can be oral, with sustained delivery over hours, or parenteral, with sustained delivery over days, weeks or months.

Representative compounds of the invention include the compounds set forth in Table 6 below. Although these compounds are depicted in the table as salts with a particular counterion, these compounds are not limited to these particular salts. Although certain compounds of the invention can be conveniently prepared as the iodide salt, the iodide anion can be exchanged for another anion, as is known in the art. The compounds listed in the table can, therefore, be prepared as salts with any suitable anion or combination of anions, such as a pharmaceutically acceptable anion, including chloride, bromide, acetate, citrate or phosphate. Similarly, Compound 56 is shown with an ammonium cation, but can be prepared with any suitable cation, preferably a pharmaceutically acceptable cation. Thus, the depiction of the compounds in the table is intended to include salts with any suitable counterion.

TABLE 6

| Compound | |
|---|---|
| Number | |
| 1 | 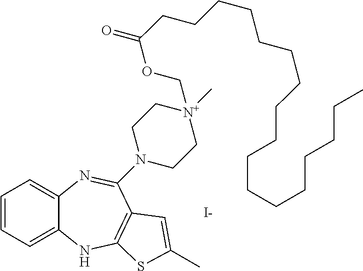 |
| 2 | 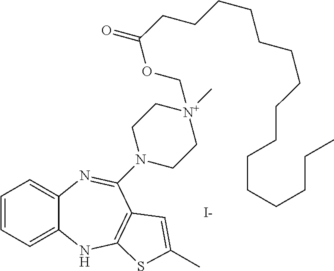 |
| 3 | 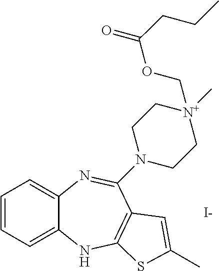 |
| 4 | 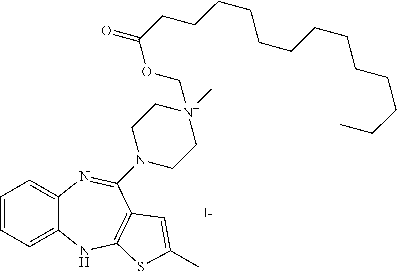 |
| 5 | 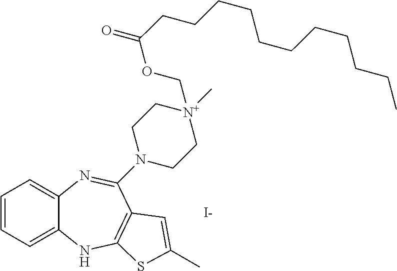 |
| 6 | 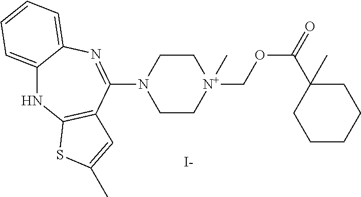 |
| 7 | 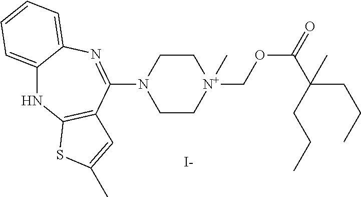 |
| 8 | 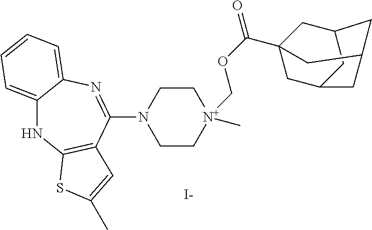 |
| 9 | 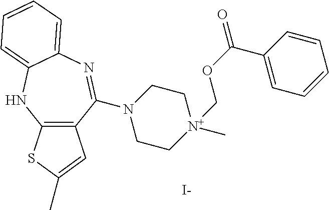 |
| 10 | 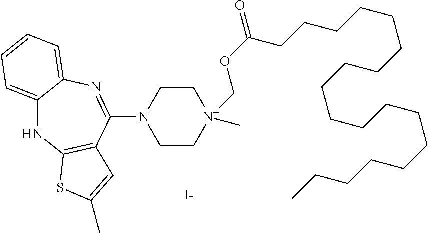 |
| 11 | 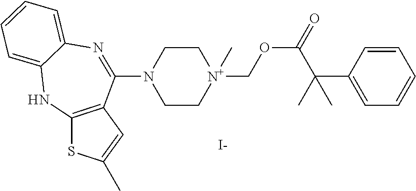 |
| 12 | 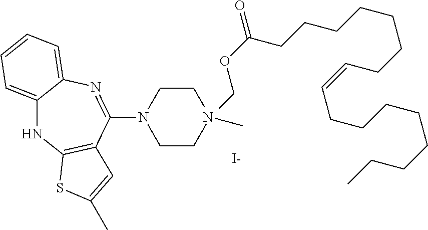 |
| 13 | 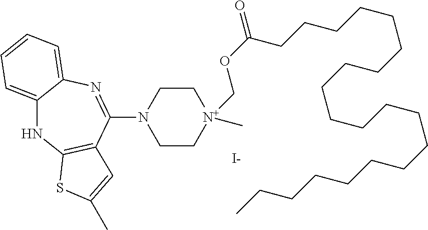 |
| 14 | 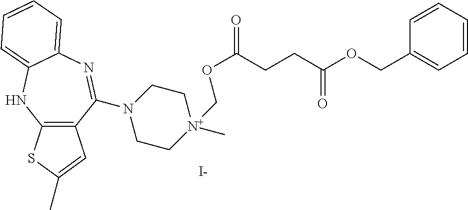 |
| 15 | 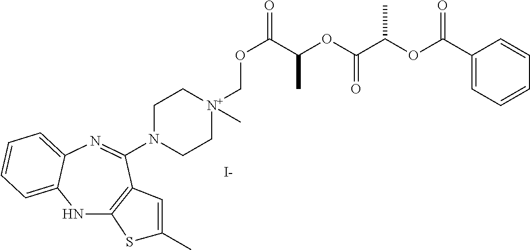 |
| 16 | 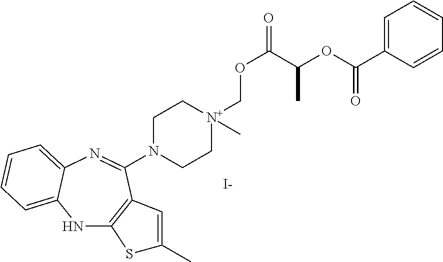 |
| 17 | 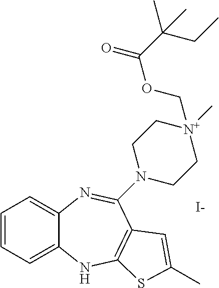 |
| 18 | 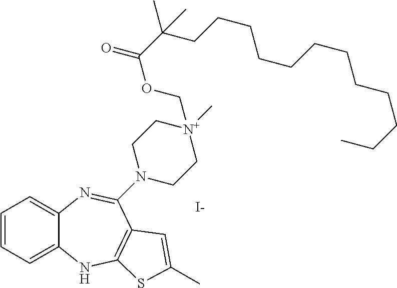 |
| 19 | 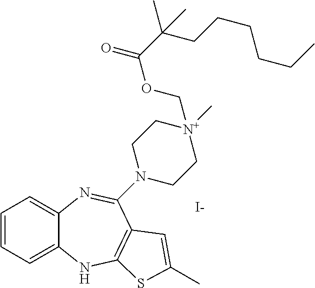 |
| 20 | 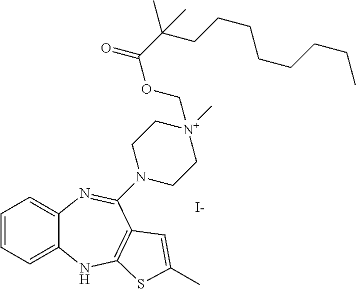 |
| 21 | 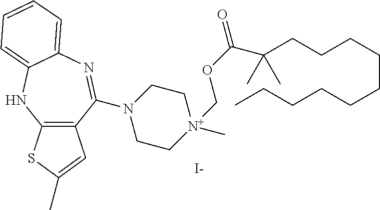 |
| 22 | 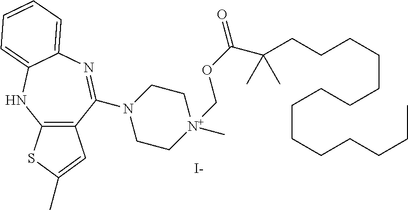 |
| 23 | 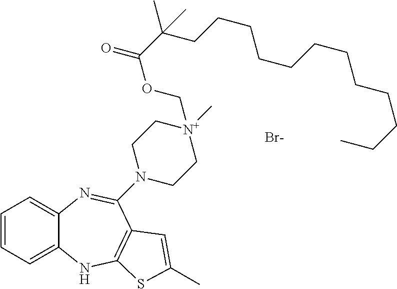 |
| 24 | 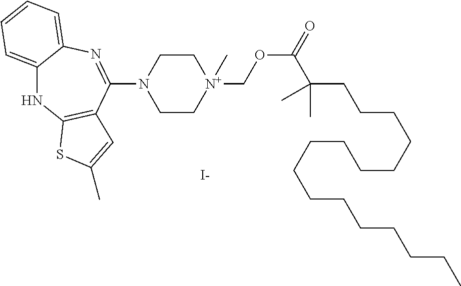 |
| 25 | 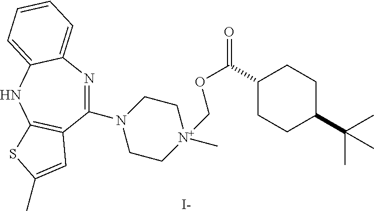 |
| 26 | 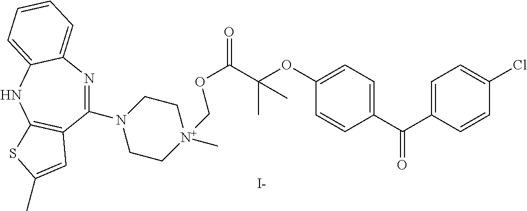 |
| 27 | 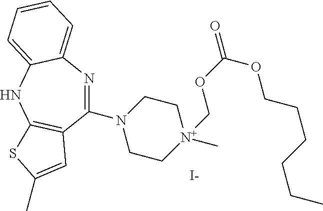 |
| 28 | 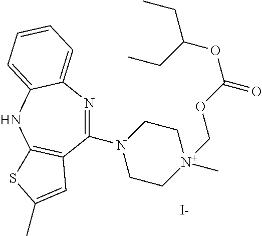 |
| 29 | 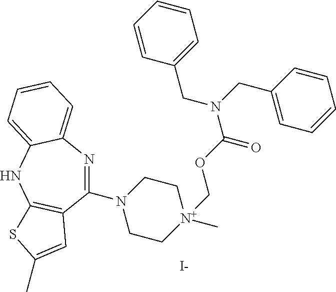 |
| 30 | 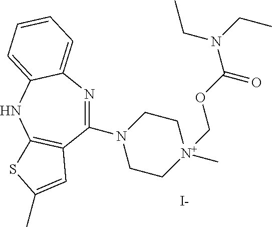 |
| 31 | 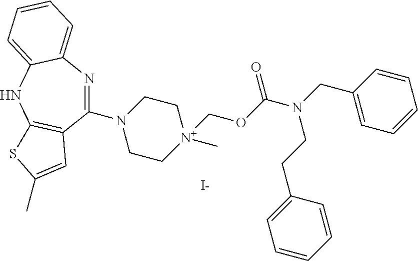 |
| 32 | 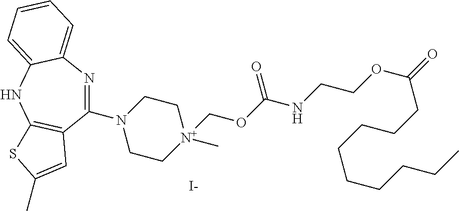 |
| 33 | 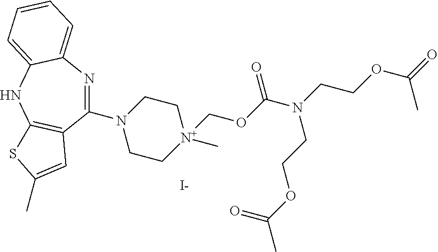 |
| 34 | 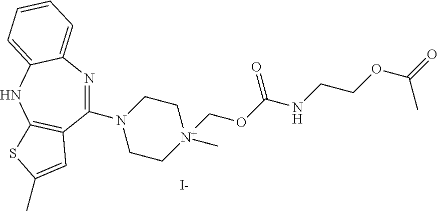 |
| 35 | 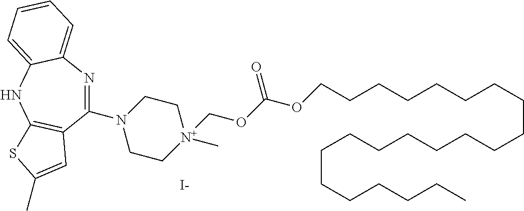 |
| 36 | 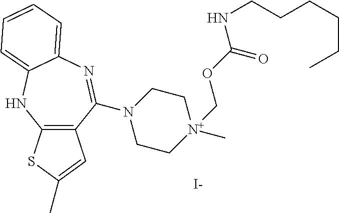 |
| 37 | 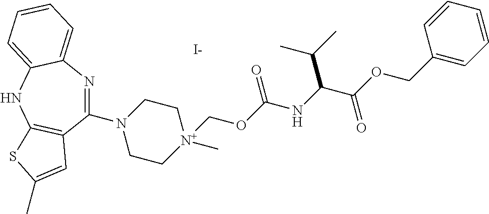 |
| 38 | 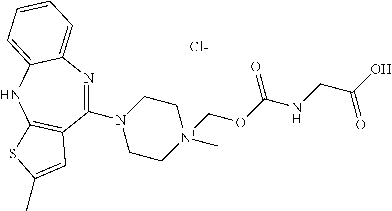 |
| 39 | 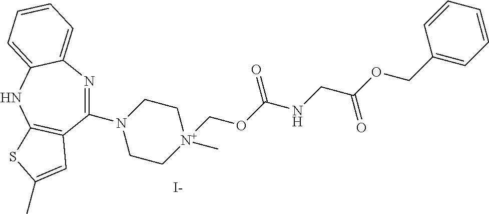 |
| 40 | 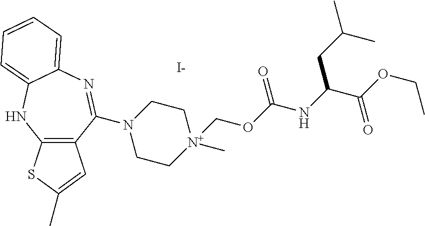 |
| 41 | 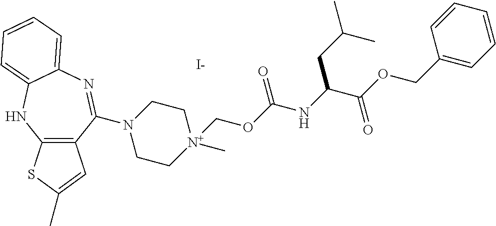 |
| 42 | 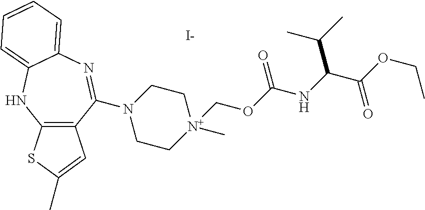 |
| 43 | 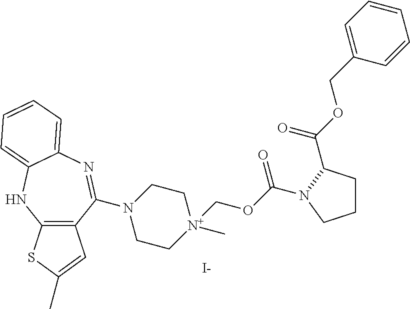 |
| 44 | 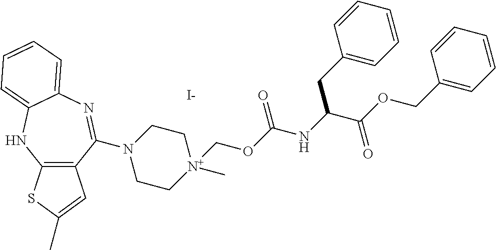 |
| 45 | 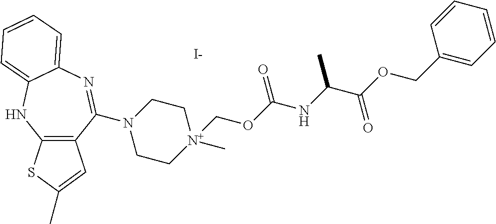 |
| 46 | 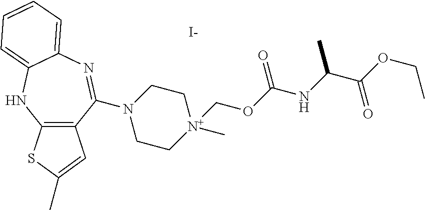 |
| 47 | 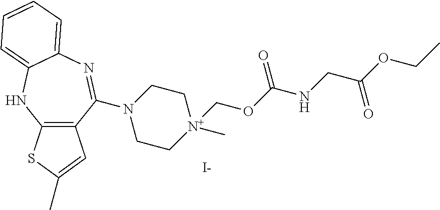 |
| 48 | 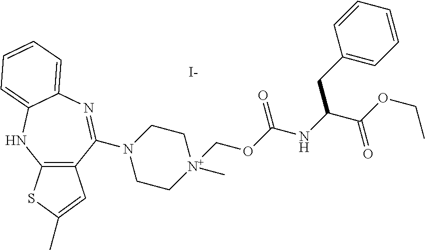 |
| 49 | 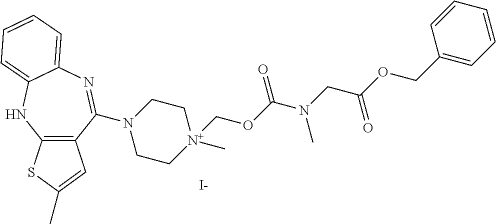 |
| 50 | 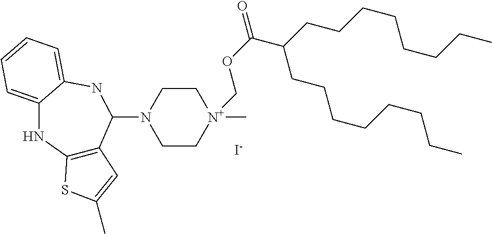 |
| 51 | 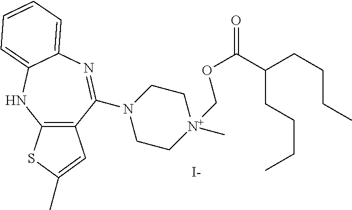 |
| 52 | 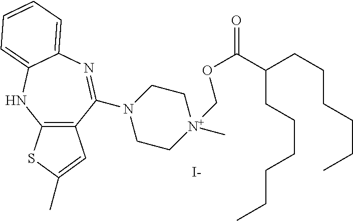 |
| 53 | 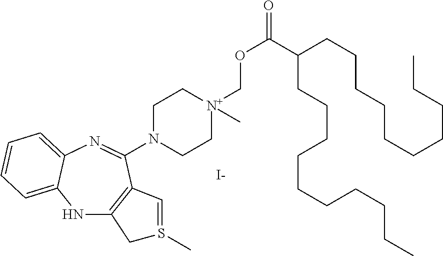 |
| 54 | 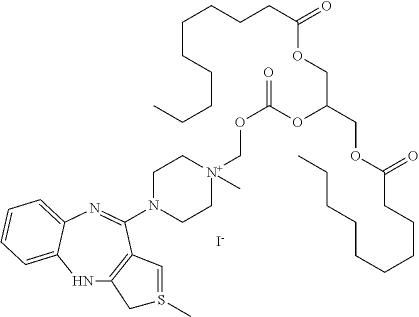 |
| 55 | 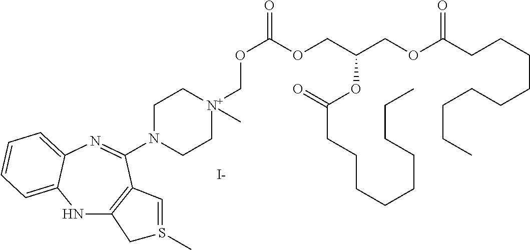 |
| 56 | 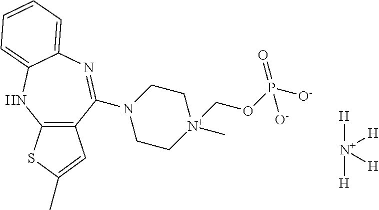 |
| 57 | 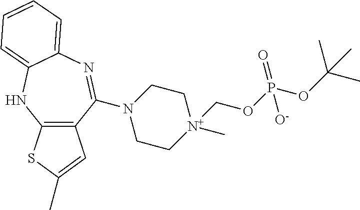 |
| 58 | 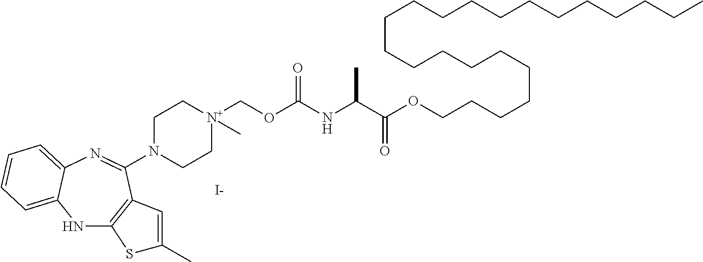 |

### DEFINITIONS

Listed below are definitions of various terms used to describe this invention. These definitions apply to the terms as they are used throughout this specification and claims, unless otherwise limited in specific instances, either individually or as part of a larger group.

The term “aliphatic group” or “aliphatic” refers to a non-aromatic moiety that may be saturated (e.g. single bond) or contain one or more units of unsaturation, e.g., double and/or triple bonds. An aliphatic group may be straight chained, branched or cyclic, contain carbon, hydrogen or, optionally, one or more heteroatoms and may be substituted or unsubstituted. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may include alkyl, substituted alkyl, alkenyl, substituted alkenyl, alkynyl, substituted alkynyl, and substituted or unsubstituted cycloalkyl groups as described herein.

The term “acyl” refers to a carbonyl substituted with hydrogen, alkyl, partially saturated or fully saturated cycloalkyl, partially saturated or fully saturated heterocycle, aryl, or heteroaryl. For example, acyl includes groups such as (C₁-C₆) alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” is intended to include both branched and straight chain, substituted or unsubstituted, saturated aliphatic hydrocarbon radicals/groups having the specified number of carbons. Preferred alkyl groups comprise about 1 to about 24 carbon atoms (“C₁-C₂₄”) preferably about 7 to about 24 carbon atoms (“C₇-C₂₄”), preferably about 8 to about 24 carbon atoms (“C₈-C₂₄”), preferably about 9 to about 24 carbon atoms (“C₉-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl” refers to heterocyclo-substituted alkyl radicals. More preferred heterocycloalkyl radicals are “lower heterocycloalkyl” radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are “lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms “aralkyl” or “arylalkyl” refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals. Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The terms “halogen” or “halo” as used herein, refers to an atom selected from fluorine, chlorine, bromine and iodine.

The terms “compound”, “drug”, and “prodrug” as used herein all include the compounds, drugs and prodrugs having the formulas disclosed herein. The compounds of the invention can occur in forms including pharmaceutically acceptable salts, solvates, hydrates, crystalline forms, amorphous forms, polymorphs, enantiomers, diastereoisomers, racemates and the like.

As used herein, the term “effective amount of the subject compounds,” with respect to the subject method of treatment, refers to an amount of the subject compound which, when delivered as part of desired dose regimen, brings about management of the disease or disorder to clinically acceptable standards.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

The following non-limiting examples are illustrative of the invention.

### Synthesis of Compounds

The compounds of the invention can be synthesized by the two methods set forth in Schemes 1 and 2 respectively. While these schemes illustrate the synthesis of compounds of Formula I and Formula III, the same synthetic methods can be applied to the synthesis of compounds of Formula II and Formula IV, respectively.

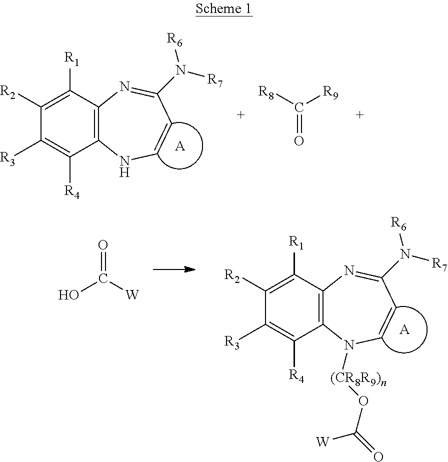

Scheme 1 illustrates the synthesis of a compound of Formula I by condensation of the parent drug compound with an aldehyde or ketone and a carboxylic, carbamic, or carbonic acid. W is R₁₀, —N(R₁₁)R₁₂ or —OR₁₀.

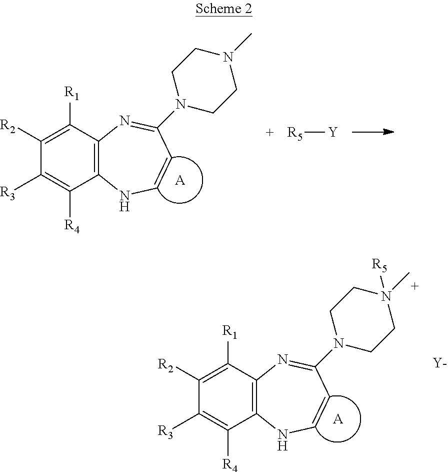

Scheme 2 illustrates the synthesis of a compound of Formula III by direct alkylation of the parent drug compound in the presence of a base. R₅ has the meaning previously given for this variable and Y is a suitable leaving group, for example, halide, such as chloride, bromide, iodide, or a sulfonate, such as triflate, methanesulfonate, mesylate, tosylate, p-bromophenylsulfonate and others as are known in the art. The process can further include anion exchange to replace Y⁻ with a desired pharmaceutically acceptable anion, X⁻.

### EXAMPLES

### Example 1

### 1-methyl-4-(2-methyl-10H-benzo[b]thieno[2,3-e][1,4]diazepin-4-yl)-1-((stearoyloxy)methyl)piperazin-1-ium iodide (Compound 1)

### A. Formation of Acid Chloride

To a stirred suspension of stearic acid (20 g, 70.3 mmol) in dichloromethane (100 mL) was added oxalyl chloride (8.92 mL, 105.5 mmol). 1 drop dimethylformamide was added and the reaction stirred at room temperature for 3 hours. The solvent was removed in vacuo and the resulting product used in the next step without further purification.

¹H-NMR (CDCl₃) δ 2.87 (t, 2H), 1.65-1.70 (m, 2H), 1.20-1.40 (m, 28H), 0.87 (3H, t).

### B. Formation of Chloromethyl Alkyl Ester

Paraformaldehyde (2.11 g, 70.3 mmol) and zinc chloride (258 mg) were added to the acid chloride prepared above and the reaction mixture was heated at 65° C. for 16 hours and then allowed to cool to room temperature. Dichloromethane (200 mL) and saturated aqueous NaHCO₃ (70 mL) were added. The aqueous emulsion was extracted with dichloromethane (2×50 mL) and the combined organic extracts washed with saturated aqueous NaHCO₃ (70 mL), brine (70 mL), and dried over MgSO₄. After filtration, the volatiles were removed and the residue purified by silica chromatography eluting with heptane to 12% dichloromethane/heptane to give a yellow solid (12.64 g, 54% yield over two steps).

¹H-NMR (CDCl₃) δ 5.70 (s, 2H), 2.37 (t, 2H), 1.55-1.70 (m, 2H), 1.20-1.40 (m, 28H) 0.86 (t, 3H).

### C. Formation of Iodomethyl Alkyl Ester

To a solution of the chloromethyl alkyl ester (12.64 g, 37.96 mmol) in acetonitrile (150 mL) and dichloromethane (75 mL) was added sodium iodide (17.07 g, 113.9 mmol). The flask was covered in tin foil to exclude light and stirred at room temperature for 70 hours and then at 25° C. for 24 hours. The reaction mixture was partitioned between dichloromethane (200 mL) and water (150 mL). The aqueous layer was extracted with dichloromethane (2×150 mL). The combined organics were washed with aq satd NaHCO₃ (200 mL), 5% aq sodium sulfite solution (200 mL) and brine (2×100 mL), then dried (MgSO₄) and concentrated to give the product as a yellow solid (14.53 g, 90% yield) which was not further purified.

¹H-NMR (CDCl₃) δ 5.90 (s, 2H), 2.32 (t, 2H), 1.55-1.70 (m, 2H), 1.20-1.35 (m, 28H), 0.87 (t, 3H).

### D. Quaternization Reaction

To a stirred solution of olanzapine (1 g, 3.20 mmol) in ethyl acetate (70 mL) was added a suspension of the iodomethyl alkyl ester (1.426 g, 3.361 mmol) in ethyl acetate (30 mL). The resultant solution was stirred at 25° C. overnight. The precipitate was collected by filtration, washed with ethyl acetate (3×10 mL), hexane (2×10 mL) and dried under vacuum to give Compound 1 (1.76 g, endotherm peak in the DSC at 160.7° C.) as a yellow solid.

¹H-NMR (CDCl₃) δ 7.02-6.89 (3H, m), 6.72 (1H, d), 6.37 (1H, s), 5.80 (2H, s), 5.54 (1H, s), 4.02-3.90 (2H, m), 3.83-3.63 (6H, m), 3.53 (3H, s), 2.51 (2H, t), 2.31 (3H, s), 1.69-1.56 (2H, m), 1.31-1.22 (28H, m), 0.87 (3H, t).

The compounds of Examples 2-9 were synthesized according to the general method of Example 1 using the appropriate acid (starting from step A) or acid chloride (starting from Step B) in place of stearic acid or stearyl chloride

### Example 2

### 1-methyl-4-(2-methyl-10H-benzo[b]thieno[2,3-e][1,4]diazepin-4-yl)-1-((palmitoyloxy)methyl)piperazin-1-ium iodide (Compound 2)

This compound was synthesized employing palmitoyl chloride. The product precipitated from the reaction mixture to give Compound 2 (1.23 g, endotherm peak in the DSC at 164° C.).

¹H-NMR (CDCl₃) δ 7.02-6.89 (3H, m), 6.67 (1H, dd), 6.35 (1H, s), 5.83 (2H, s), 5.32 (1H, s), 4.03-3.96 (2H, m), 3.79-3.71 (6H, m), 3.56 (3H, s), 2.52 (2H, t), 2.31 (3H, s), 1.64 (2H, t), 1.39-1.21 (24H, m), 0.87 (3H, t).

### Example 3

### 1-((butyryloxy)methyl)-1-methyl-4-(2-methyl-10H-benzo[b]thieno[2,3-e][1,4]diazepin-4-yl)piperazin-1-ium iodide (Compound 3)

This compound was synthesized employing butyryl chloride instead of stearyl chloride. The final precipitated from the reaction mixture to give Compound 3 (1.8 g, endotherm peak in the DSC at 203.2° C.).

¹H-NMR (d₆-DMSO) δ 6.77-6.88 (m, 3H), 6.65-6.69 (m, 1H), 6.37 (s, 1H), 5.42 (s, 2H), 3.78-3.89 (m, 2H), 3.45-3.60 (m, 6H), 3.16 (s, 3H), 2.51 (t, 2H), 2.25 (s, 3H), 1.57 (st, 2H), 0.89 (t, 3H).

### Example 4

### 1-methyl-4-(2-methyl-10H-benzo[b]thieno[2,3-e][1,4]diazepin-4-yl)-1-((tetradecanoyloxy)methyl)piperazin-1-ium iodide (Compound 4)

This compound was synthesized employing myristoyl chloride. The final product precipitated from the reaction mixture to give Compound 4 (2.83 g, 93%, endotherm peak in the DSC at 153° C.).

¹H-NMR (CDCl₃) δ 7.00-6.92 (3H, m), 6.73 (1H, d), 6.37 (1H, s), 5.80 (2H, s), 5.62 (NH), 4.01-3.93 (2H, m), 3.82-3.69 (6H, m), 3.53 (3H, s), 2.51 (2H, t), 2.31 (3H, s), 1.75-1.58 (2H, m), 1.32-1.20 (22H, m), 0.87 (3H, t).

### Example 5

### 1-((dodecanoyloxy)methyl)-1-methyl-4-(2-methyl-10H-benzo[b]thieno[2,3-e][1,4]diazepin-4-yl)piperazin-1-ium iodide (Compound 5)

This compound was synthesized employing lauroyl chloride. The final product precipitated from the reaction mixture to give Compound 5 (1.12 g, 67%, endotherm peak in the DSC at 151° C.).

¹H-NMR (300 MHz, d₆-DMSO) δ 7.73 (1H, s), 6.86-6.77 (3H, m), 6.69-6.65 91H, m), 6.37 (1H, s), 5.41 (2H, s), 3.85-3.76 (2H, m), 3.56-3.45 (6H, m), 3.16 (3H, s), 2.53 (2H, t), 2.25 (3H, s), 1.58-1.52 (2H, m), 1.29-1.18 (10H, m), 0.82 (3H, t).

### Example 6

### 1-methyl-4-(2-methyl-10H-benzo[b]thieno[2,3-e][1,4]diazepin-4-yl)-1-(((1-methylcyclohexanecarbonyl)oxy)methyl)piperazin-1-ium iodide (Compound 6)

This was compound was synthesized employing 1-methylcyclohexanecarbonyl chloride. The final product precipitated from the reaction mixture to give Compound 6 (2.56 g, 96%, endotherm peak in the DSC at 168.2° C.).

¹H-NMR (CDCl₃) δ 7.00-6.90 (3H, m), 6.69 (1H, d), 6.37 (1H, s), 5.80 (2H, s), 5.47 (NH), 4.07-3.96 (2H, m), 3.83-3.72 (6H, m), 3.57 (3H, s), 2.31 (3H, s), 2.01-1.92 (2H, m), 1.78-1.50 (6H, m), 1.50-1.20 (9H, m).

### Example 7

### 1-Methyl-4-(2-methyl-10H-benzo[b]thieno[2,3-e][1,4]diazepin-4-yl)-1-(((2-methyl-2-propylpentanoyl)oxy)methyl)piperazin-1-ium iodide (Compound 7)

This compound was synthesized employing 2-methyl-2-propylpentanoic acid. The final product precipitated from the reaction mixture to give Compound 7 (2.12 g, 78%, endotherm peak in the DSC at 189° C.).

¹H-NMR (DMSO-d₆) δ 6.88-6.69 (3H, m), 6.70-6.65 (1H, m), 6.34 (1H, s), 5.43 (2H, s), 3.90-3.80 (2H, m), 3.60-3.49 (6H, m), 3.18 (3H, s), 2.25 (3H, s), 1.59 (2H, dt), 1.43 (2H, dt), 1.30-1.05 (7H, m), 0.83 (6H, t).

### Example 8

### 1-((((3 r, 5 r, 7 r)-Adamantane-1-carbonyl)oxy)methyl)-1-methyl-4-(2-methyl-10H-benzo[b]thieno[2,3-e][1,4]diazepin-4-yl)piperazin-1-ium iodide (Compound 8)

This compound was synthesized employing 1-adamantane carboxylic acid. The final product precipitated from the reaction mixture to give Compound 8 (2.12 g, 78%, endotherm peak in the DSC at 189° C.).

¹H-NMR (300 MHz, d₆-DMSO) δ 7.75 (NH), 6.88-6.78 (3H, m), 6.68-6.65 (1H, m), 6.35 (2H, s), 3.90-3.78 (2H, m), 3.60-3.42 (6H, m), 3.18 (3H, s), 2.25 (3H, s), 1.97-1.90 (3H, m), 1.90-1.85 (6H, m), 1.68-1.58 (6H, m).

### Example 9

### 1-((Benzoyloxy)methyl)-1-methyl-4-(2-methyl-10H-benzo[b]thieno[2,3-e][1,4]diazepin-4-yl)piperazin-1-ium iodide (Compound 9)

This compound was synthesized employing benzoyl chloride. The final product precipitated from the reaction mixture to give Compound 9 (2.97 g, 85%, endotherm peak in the DSC at 164.7° C.).

¹H-NMR (300 MHz, d₆-DMSO) δ 8.12 (2H, d), 7.75 (2H, t), 7.57 (2H, t), 6.87-6.76 (3H, m), 6.71-6.64 (1H, m), 6.40 (1H, s), 5.66 (2H, s), 3.92-3.83 (2H, m), 3.76-3.51 (6H, m), 3.30 (3H, s), 2.26 (3H, s).

### Example 10

### 1-((eicosanoyloxy)methyl)-1-methyl-4-(2-methyl-10H-benzo[b]thieno[2,3-e][1,4]diazepin-4-yl)piperazin-1-ium iodide (Compound 10)

To a suspension of arachidic acid (8 g, 25.6 mmol) in water (80 mL) was added Na₂CO₃ (10.9 g, 102.4 mmol). After 20 minutes, the reaction was cooled to 0° C. and nBu₄NHSO₄ (1.74 g, 5.12 mmol), dichloromethane (160 mL) and chloromethyl chlorosulfate (3.4 mL, 33.3 mmol) were added. The reaction was allowed to warm to 25° C. and stirred overnight. The reaction mixture was separated and the aqueous extracted with dichloromethane (2×200 mL). The combined organics were dried (MgSO₄) and concentrated in vacuo. The product was purified by column chromatography eluting with heptane to 10% dichloromethane/heptane to give the product (6.54 g, 71%).

The iodomethyl ester formation and quaternisation reactions were then carried out as described in steps C and D of Example 1 using arachidoyl chloride instead of stearoyl chloride. The final product precipitated from the reaction mixture to give Compound 10 (2.77 g, 87%, endotherm peak in the DSC at 149° C.).

¹H-NMR (300 MHz, CDCl₃) δ 7.01-6.89 (3H, m), 6.70 (1H, d), 6.37 (1H, s), 5.81 (2H, s), 5.48 (1H, s), 4.03-3.91 (2H, m), 3.79-3.70 (6H, m), 3.54 (3H, s), 2.51 (2H, t), 2.31 (3H, s), 1.69-1.61 (2H, m), 1.35-1.19 (35H, m), 0.87 (3H, t).

The compounds of Examples 11-18 were prepared using the general method of Example 10 employing the appropriate acid.

### Example 11

### 1-Methyl-4-(2-methyl-10H-benzo[b]thieno[2,3-e][1,4]diazepin-4-yl)-1-(((2-methyl-2-phenylpropanoyl)oxy)methyl)piperazin-1-ium iodide (Compound 11)

This compound was synthesized according to the general procedure of Example 10 employing 2-methyl-2-phenylpropanoic acid. The product precipitated from the reaction to give Compound 11 (2.29 g, 66%, endotherm peak in the DSC at 193.8° C.).

¹H-NMR (300 MHz, d₆-DMSO) δ 7.74 (NH), 7.42-7.31 (4H, m), 7.30-7.22 (1H, m), 7.88-7.78 (3H, m), 6.70-6.65 (1H, m), 6.31 (1H, s), 5.42 (2H, s), 3.80-3.72 (2H, m), 3.54-3.30 (6H, m), 3.01 (3H, s), 2.26 (3H, s), 1.60 (6H, s).

### Example 12

### (Z)-1-Methyl-4-(2-methyl-10H-benzo[b]thieno[2,3-e][1,4]diazepin-4-yl)-1-((oleoyloxy)methyl)piperazin-1-ium iodide (Compound 12)

This compound was synthesized according to the general procedure of Example 10 employing oleic acid. The final product precipitated from the reaction to give Compound 12 (2.28 g, 65%, endotherm peak in the DSC at 164.7° C.).

¹H-NMR (300 MHz, CDCl₃) δ 7.00-6.90 (3H, m), 6.71-6.66 (1H, m), 6.36 (1H, s), 5.82 (2H, s), 5.45-5.30 (3H, m), 4.04-3.93 (2H, m), 3.80-3.68 (6H, m), 3.54 (3H, s), 2.52 (2H, t), 2.31 (3H, s), 2.05-1.95 (4H, m), 1.69-1.58 (2H, m), 1.35-1.20 (12H, m), 0.87 (3H, t).

### Example 13

### 1-((docosanoyloxy)methyl)-1-methyl-4-(2-methyl-10H-benzo[b]thieno[2,3-e][1,4]diazepin-4-yl)piperazin-1-ium iodide (Compound 13)

This compound was synthesized according to the general procedure of Example 10 employing docosanoic acid. The product precipitated from the reaction to give Compound 13 (4.21 g, 84%, endotherm peak in the DSC at 146° C.).

¹H-NMR (300 MHz, CDCl₃) δ 7.00-6.92 (3H, m), 6.71-6.66 (1H, m), 6.36 (1H, s), 5.82 (2H, s), 5.43 (NH), 4.01-3.93 (2H, m), 3.82-3.68 (6H, m), 3.54 (3H, s), 2.51 (2H, t), 2.31 (3H, s), 1.67-1.60 (2H, m), 1.32-1.22 (36H, m), 0.87 (3H, t).

### Example 14

### 1-(((4-(benzyloxy)-4-oxobutanoyl)oxy)methyl)-1-methyl-4-(2-methyl-10H-benzo[b]thieno[2,3-e][1,4]diazepin-4-yl)piperazin-1-ium iodide (Compound 14)

### Synthesis of 4-(benzyloxy)-4-oxobutanoic acid

Succinic anhydride (7 g, 70.0 mmol) and benzyl alcohol (8.7 mL, 83.9 mmol) were combined in dichloromethane (350 mL) at 0° C. and DMAP (0.85 g, 7.0 mmol) was added portion-wise. The reaction was allowed to gradually warm to 25° C. and stirred for 4 days. The reaction mixture was washed with 1M HCl (3×200 mL) then water (300 mL). The organic phases were then extracted with aq saturated NaHCO₃ (3×300 mL). This was then acidified with conc HCl until pH 1 resulting in a solid precipitating which was filtered then dissolved in dichloromethane. The dichloromethane was dried (MgSO₄) and concentrated in vacuo to give 4-(benzyloxy)-4-oxobutanoic acid (10.36 g, 71%).

¹H-NMR (300 MHz, CDCl₃) δ 7.41-7.29 (5H, m), 5.15 (2H, s), 2.74-2.63 (4H, m).

Compound 14 was synthesized according to the general procedure of Example 10 employing 4-(benzyloxy)-4-oxobutanoic acid. The product precipitated from the reaction to give Compound 14 (1.80 g, 85%).

¹H-NMR (300 MHz, CDCl₃) δ 7.39-7.27 (5H, m), 7.02-6.95 (3H, m), 6.72 (1H, d), 6.38 (1H, s), 5.89 (2H, s), 5.12 (2H, s), 4.02-3.64 (8H, m), 3.40 (3H, s), 2.79 (4H, s), 2.32 (3H, s).

### Example 15

### 1-((((S)-2-(((S)-2-(benzoyloxy)propanoyl)oxy)propanoyl)oxy)methyl)-1-methyl-4-(2-methyl-10H-benzo[b]thieno[2,3-e][1,4]diazepin-4-yl)piperazin-1-ium iodide (Compound 15)

This compound was synthesized according to the general procedure of Example 10 employing (S)-1-(S)-1-(1-oxopropan-2-yloxy)-1-oxopropan-2-yl benzoic acid. The final product precipitated from the reaction to give Compound 15 (0.28 g, 15%).

¹H-NMR (300 MHz, CDCl₃) δ 8.04 (2H, d), 8.60 (1H, t), 7.45 (2H, t), 6.93-7.08 (3H, m), 6.75-6.80 (m, 1H), 6.43 (1H, s), 6.01 (1H, d), 5.90 (1H, d), 5.28 (1H, q), 5.06 (1H, q), 3.95-4.15 (4H, m), 3.70-3.95 (4H, m), 3.47 (3H, s), 3.08 (NH), 2.26 (3H, s), 1.69 (3H, d), 1.61 (3H, d).

### Example 16

### (S)-1-(((2-(benzoyloxy)propanoyl)oxy)methyl)-1-methyl-4-(2-methyl-10H-benzo[b]thieno[2,3-e][1,4]diazepin-4-yl)piperazin-1-ium iodide (Compound 16)

This compound was synthesized according to the general procedure of Example 10 employing (S)-1-(1-oxopropan-2-yl)benzoic acid. The final product precipitated from the reaction to give Example 16 (0.9 g, 36%).

¹H-NMR (300 MHz, CDCl₃) δ 8.03 (2H, d), 7.62 (1H, t), 7.47 (2H, t), 6.91-7.05 (3H, m), 6.79 (1H, d), 6.40 (1H, s), 6.01 (2H, dd), 5.15 (1H, q), 3.72-4.05 (8H, m), 3.46 (3H, s), 2.30 (3H, s), 1.72 (3H, d).

### Example 17

### 1-(((2,2-Dimethylbutanoyl)oxy)methyl)-1-methyl-4-(2-methyl-10H-benzo[b]thieno[2,3-e][1,4]diazepin-4-yl)piperazin-1-ium iodide (Compound 17)

This compound was synthesized according to the general procedure of Example 10 employing 2,2-dimethylbutyryl chloride. The product precipitated from the reaction mixture to give Compound 17 (2.27 g, endotherm peak in the DSC at 197.2° C.).

¹H-NMR (CDCl₃) δ 7.02-6.93 (3H, m), 6.67 (1H, d), 6.35 (1H, s), 5.81 (2H, s), 5.27 (1H, s), 4.03-3.95 (2H, m), 3.83-3.72 (6H, m), 3.58 (3H, s), 2.32 (3H, s), 1.65-1.61 (2H, m), 1.21 (9H, s), 0.83 (3H, t).

### Example 18

### 1-(((2,2-dimethyltetradecanoyl)oxy)methyl)-1-methyl-4-(2-methyl-10H-benzo[b]thieno[2,3-e][1,4]diazepin-4-yl)piperazin-1-ium iodide (Compound 18)

### A. Synthesis of methyl 2,2-dimethyltetradecanoate

To a stirred solution of diisopropylamine (6.90 mL, 49.0 mmol) in THF (50 mL) under Ar (g) at −7° C. was added n-BuLi (2.3M in hexanes, 21.3 mL, 49.0 mmol) dropwise via a dropping funnel keeping the temp. between 0° C. and 5° C. The reaction was stirred at −7° C. for 30 mins. and then cooled to −78° C. Methyl isobutyrate (5.61 mL, 49.0 mmol) was added and the reaction stirred at −78° C. for 1.5 hours. 1-iodododecane (13.05 g, 44.1 mmol) in THF (10 mL) was added dropwise via a dropping funnel keeping the temperature below −70° C. A further 40 mL THF was added over 5 mins. to aid stirring. After complete addition the reaction was stirred at −78° C. for approx. 2 hours and then allowed to slowly warm to room temperature overnight. The reaction was quenched with sat. aq. NH₄Cl (100 mL) and diluted with ethyl acetate (100 mL). The aqueous layer was extracted with ethyl acetate (2×50 mL) and the combined organics washed with brine (50 mL) and dried over MgSO₄. After filtration, the volatiles were removed. The reaction was repeated in a similar manner using methyl isobutyrate (15.05 mL, 131.27 mmol). The two crude batches were combined and purified by silica chromatography eluting heptane to 50% dichloromethane/heptane to give methyl 2,2-dimethyl myristate (31.7 g).

### B. Synthesis of 2,2-dimethyltetradecanoic acid

To a stirred solution of methyl 2,2-dimethyltetradecanoate (31.7 g, 117.2 mmol) in ethanol (234 mL) was added 2M NaOH (117 mL, 234.4 mmol). The reaction was stirred at room temperature overnight. NaOH (4.69 g, 117 mmol) was added and the reaction heated at 50° C. for 24 hours. NaOH (4.69 g, 117 mmol) was added and the reaction heated to 100° C. for 4 hours and then cooled to room temperature. 140 mL 4M HCl was added to acidify. Ethyl acetate (200 mL) was added and the layers separated. The aqueous was extracted with ethyl acetate (2×100 mL) and the combined organics concentrated in vacuo. The residue was partitioned between ethyl acetate (200 mL) and brine (100 mL). The organic layer was washed with brine (50 mL) and dried over MgSO₄. After filtration, the volatiles were removed to give 2,2-dimethyltetradecanoic acid (26.9 g).

### C. Synthesis of Compound 18

This compound was synthesized employing the general procedure of Example 10 employing 2,2-dimethyltetradecanoic acid. The final product precipitated from the reaction mixture to give Compound 18 (1.84 g, endotherm peak in the DSC at 177.5° C.).

¹H-NMR (CDCl₃) δ 7.01-6.89 (3H, m), 6.71-6.66 (1H, m), 6.37 (1H, s), 5.77 (2H, s), 5.40 (1H, s), 4.04-3.90 (2H, m), 3.84-3.67 (6H, m), 3.57 (3H, s), 2.31 (3H, s), 1.59-1.49 (2H, m), 1.31-1.10 (26H, m), 0.87 (3H, t).

The compounds of Examples 19-24 were prepared using the general method of Example 18, using the appropriate iodoalkane in place of 1-iodododecane.

### Example 19

### 1-(((2,2-dimethyloctanoyl)oxy)methyl)-1-methyl-4-(2-methyl-10H-benzo[b]thieno[2,3-e][1,4]diazepin-4-yl)piperazin-1-ium iodide (Compound 19)

This compound was synthesized via 2,2-dimethyloctanoic acid. The final product precipitated from the reaction mixture to give Compound 19 (2.5 g, 83%, endotherm peak in the DSC at 176° C.).

¹H-NMR (DMSO-d₆) δ 7.74 (NH), 6.88-6.76 (3H, m), 6.70-6.63 (1H, m), 6.35 (1H, s), 5.42 (2H, s), 3.90-3.75 (2H, m), 3.60-3.44 (6H, m), 3.17 (3H, s), 2.25 (3H, s), 1.54-1.46 (2H, m), 1.28-1.10 (14H, m), 0.81 (3H, t).

### Example 20

### 1-(((2,2-dimethyldecanoyl)oxy)methyl)-1-methyl-4-(2-methyl-10H-benzo[b]thieno[2,3-e][1,4]diazepin-4-yl)piperazin-1-ium iodide (Compound 20)

This compound was synthesized via 2,2-dimethyldecanoic acid. The final product precipitated from the reaction mixture to give Compound 20 (2.8 g, 90%, endotherm peak in the DSC at 157.2° C.).

¹H-NMR (DMSO-d₆) δ 7.74 (NH), 6.88-6.75 (3H, m), 6.70-6.63 (1H, m), 6.35 (s, 1H), 5.42 (s, 2H), 3.89-3.78 (2H, m), 3.60-3.45 (6H, m), 3.18 (3H, s), 2.25 (s, 3H), 1.56-1.48 (2H, m), 1.29-1.11 (16H, m), 0.81 (3H, t).

### Example 21

### 1-(((2,2-dimethyldodecanoyl)oxy)methyl)-1-methyl-4-(2-methyl-10H-benzo[b]thieno[2,3-e][1,4]diazepin-4-yl)piperazin-1-ium iodide (Compound 21)

This compound was synthesized via 2,2-dimethyldodecanoic acid. The final product precipitated from the reaction mixture to give Compound 21 (1.5 g, 69%, endotherm peak in the DSC at 141° C.).

¹H-NMR (CDCl₃) δ 7.00-6.90 (3H, m), 6.71-6.66 (1H, m), 6.37 (1H, s), 5.77 (2H, s), 5.40 (1H, s), 4.05-3.90 (2H, m), 3.80-3.67 (6H, m), 3.57 (3H, s), 2.31 (3H, s), 1.58-1.50 (2H, m), 1.30-1.10 (16H, m), 0.87 (3H, t).

### Example 22

### 1-(((2,2-dimethylhexadecanoyl)oxy)methyl)-1-methyl-4-(2-methyl-10H-benzo[b]thieno[2,3-e][1,4]diazepin-4-yl)piperazin-1-ium iodide (Compound 22)

This compound was synthesized via 2,2-dimethylhexadecanoic acid. The final product precipitated from the reaction mixture to give Compound 22 (1.92 g, 82%, endotherm peak in the DSC at 165.1° C.).

¹H-NMR (CDCl₃) δ 7.00-6.90 (3H, m), 6.67-6.62 (1H, m), 6.34 (1H, s), 5.80 (2H, s), 5.22 (NH), 4.02-3.95 (2H, m), 3.81-3.70 (6H, m), 3.57 (3H, s), 2.31 (3H, s), 1.52-1.60 (2H, m), 1.30-1.13 (30H, m), 0.87 (3H, t).

### Example 23

### 1-(((2,2-dimethyltetradecanoyl)oxy)methyl)-1-methyl-4-(2-methyl-10H-benzo[b]thieno[2,3-e][1,4]diazepin-4-yl)piperazin-1-ium bromide (Compound 23)

This compound was synthesized via 2,2-dimethyloctadecanoic acid. NaI was replaced with NaBr. The final product precipitated from the reaction mixture to give Compound 23 (1.28 g, 59%, endotherm peak in the DSC at 186° C.).

¹H-NMR (CDCl₃) δ 7.02-6.90 (3H, m), 6.63 (1H, d), 6.30 (1H, s), 5.89 (2H, s), 5.21 (NH), 4.03-3.95 (2H, m), 3.85-3.68 (6H, m), 3.58 (3H, s), 2.31 (3H, s), 1.60-1.52 (2H, m), 1.32-1.14 (26H, m), 0.87 (3H, t).

### Example 24

### 1-(((2,2-dimethyloctadecanoyl)oxy)methyl)-1-methyl-4-(2-methyl-10H-benzo[b]thieno[2,3-e][1,4]diazepin-4-yl)piperazin-1-ium iodide (Compound 24)

This compound was synthesized via 2,2-dimethyloctadecanoic acid. The final product precipitated from the reaction mixture to give Compound 24 (2.91 g, 92%, endotherm peak in the DSC at 136.7° C.).

¹H-NMR (CDCl₃) δ 7.00-6.92 (3H, m), 6.71-6.66 (1H, m), 6.36 (1H, s), 5.78 (2H, s), 5.44 (NH), 4.06-3.95 (2H, m), 3.81-3.70 (6H, m), 3.55 (3H, s), 2.31 (3H, s), 1.58-1.50 (2H, m), 1.30-1.12 (34H, m), 0.87 (3H, t).

### Example 25

### 1-((((1 r, 4 r)-4-(tert-butyl)cyclohexanecarbonyl)oxy)methyl)-1-methyl-4-(2-methyl-10H-benzo[b]thieno[2,3-e][1,4]diazepin-4-yl)piperazin-1-ium iodide (Compound 25)

This compound was synthesized via the general method of Example 10 using 4-tert-butylcyclohexanecarboxylic acid. The final product precipitated from the reaction mixture to give Compound 25 (2.81 g, 84%, endotherm peak in the DSC at 185.3° C.).

¹H-NMR (300 MHz, CDCl₃) δ 7.01-6.89 (3H, m), 6.94 (1H, d), 6.32 (1H, s), 5.84 (2H, s), 5.12 (1H, s), 4.05-3.99 (2H, m), 3.75-3.66 (6H, m), 3.58 (3H, s), 02.41-2.33 (1H, m), 2.32 (3H, s), 2.07-2.01 (2H, m), 1.78-1.72 (2H, m), 1.43-1.33 (2H, m), 1.03-0.92 (3H, m), 0.81 (9H, s).

### Example 26

### 1-(((2-(4-(4-chlorobenzoyl)phenoxy)-2-methylpropanoyl)oxy)methyl)-1-methyl-

### 4-(2-methyl-10H-benzo[b]thieno[2,3-e][1,4]diazepin-4-yl)piperazin-1-ium iodide (Compound 26)

This compound was prepared using the general method of Example 10 employing 2-[4-(4-Chlorobenzoyl)-phenoxy]-2-methylpropionic acid. The quaternization reaction was conducted in cyclopropyl methyl ether. The final product precipitated from the reaction and was purified by dissolution in a minimum amount of dichloromethane followed by precipitation with ethyl acetate to give Compound 26 (2.08 g, 57%, endotherm peak in the DSC at 175.2° C.).

¹H-NMR (300 MHz, CDCl₃) δ 7.78 (2H, d), 7.71 (2H, d), 7.01-6.89 (5H, m), 6.81-6.62 (1H, m), 6.33 (1H, s), 6.04 (2H, s), 5.39 (1H, br s), 4.06-3.92 (2H, m), 3.79-3.59 (6H, m), 3.44 (3H, s), 2.29 (3H, s), 1.74 (6H, s).

### Example 27

### 1-((((Hexyloxy)carbonyl)oxy)methyl)-1-methyl-4-(2-methyl-10H-benzo[b]thieno[2,3-e][1,4]diazepin-4-yl)piperazin-1-ium iodide (Compound 27)

To a solution of chloromethyl chloroformate (9.6 mL, 107.7 mmol) in dichloromethane (100 mL) at 0° C. was added a solution of 1-hexanol (10 g, 97.9 mmol) and pyridine (8.7 mL, 107.7 mmol) in dichloromethane (25 mL) dropwise over 3 hours (keeping the temp at approx 0° C.). The reaction was allowed to gradually warm to 25° C. overnight. 1M HCl (50 ml) was added to the reaction mixture and separated. The organics were washed with 1M HCl (50 mL), water (100 mL), aq satd NaHCO₃ (2×100 mL), brine (100 mL) and dried (MgSO₄) to give hexyl chloromethyl carbonate (18.53 g, 97%).

Compound 27 was prepared via steps C and D of the general method of Example 1 using hexyl chloromethyl carbonate. The product precipitated from the reaction and was re-triturated by dissolving in a minimum amount of dichloromethane and precipitated with diethyl ether to give Compound 28 (2.13 g, 86%, endotherm peak in the DSC at 140° C.).

¹H-NMR (300 MHz, CDCl₃) δ 7.04-6.92 (3H, m), 6.66 (1H, d), 6.37 (1H, s), 5.92 (2H, s), 5.29 (1H, s), 4.25 (2H, t), 4.08-3.94 (2H, m), 3.88-3.69 (6H, m), 3.56 (3H, s), 2.32 (3H, s), 1.77-1.51 (4H, m), 1.43-1.26 (4H, m), 0.90 (3H, t).

The compounds of Examples 28-49 were prepared using the general method of Example 27 using the appropriate carbonate or carbamate in place of hexyl chloromethyl carbonate.

### Example 28

### 1-Methyl-4-(2-methyl-10H-benzo[b]thieno[2,3-e][1,4]diazepin-4-yl)-1-((((pentan-3-yloxy)carbonyl)oxy)methyl)piperazin-1-ium iodide (Compound 28)

This compound was synthesized via iodomethyl pentan-3-yl carbonate. The final product precipitated from the reaction mixture to give Compound 28 (2.93 g, 87%, endotherm peak in the DSC at 169.9° C.).

¹H-NMR (300 MHz, d₆-DMSO) δ 7.73 (1H, s), 6.85-6.78 (3H, m), 6.69-6.64 (1H, m), 6.37 (1H, s), 5.45 (2H, s), 4.64-4.56 (1H, m), 3.88-3.79 (2H, m), 3.56-3.48 (6H, m), 3.18 (3H, s), 2.25 (3H, s), 1.69-1.53 (4H, m), 0.85 (6H, t).

### Example 29

### 1-(((dibenzylcarbamoyl)oxy)methyl)-1-methyl-4-(2-methyl-10H-benzo[b]thieno[2,3-e][1,4]diazepin-4-yl)piperazin-1-ium iodide (Compound 29)

This compound was synthesized via iodomethyl dibenzyl carbamate. The product precipitated from the reaction and was purified by trituration with diethyl ether/dichloromethane, 1:2 to give Compound 29 (2.29 g, 79%).

¹H-NMR (300 MHz, d₆-DMSO) δ 7.73 (1H, s), 7.38-7.22 (8H, m), 6.87-6.78 (4H, m), 6.68-6.64 (1H, m), 6.34 (1H, s), 5.44 (2H, s), 4.53 (4H, s), 3.81-3.75 (2H, m), 3.53-3.31 (6H, m), 2.99 (3H, s), 2.26 (3H, s).

### Example 30

### 1-(((Diethylcarbamoyl)oxy)methyl)-1-methyl-4-(2-methyl-10H-benzo[b]thieno[2,3-e][1,4]diazepin-4-yl)piperazin-1-ium iodide (Compound 30)

This compound was synthesized via iodomethyl diethyl carbamate. The final product precipitated from the reaction mixture to give Compound 30 (3.10 g, 95%, endotherm peak in the DSC at 205.6° C.).

¹H-NMR (300 MHz, d₆-DMSO) δ 7.73 (1H, s), 6.86-6.79 (2H, m), 6.69-6.61 (1H, m), 6.37 (1H, s), 5.39 (2H, s), 3.61-3.46 (6H, m), 3.29-3.21 (4H, m), 3.14 (3H, s), 2.25 (3H, s), 1.14-1.01 (6H, m).

### Example 31

### 1-(((Benzyl(phenethyl)carbamoyl)oxy)methyl)-1-methyl-4-(2-methyl-10H-benzo[b]thieno[2,3-e][1,4]diazepin-4-yl)piperazin-1-ium iodide (Compound 31)

This compound was synthesized via iodomethyl benzyl(phenethyl)carbamate. The final product precipitated from the reaction mixture to give Compound 31 (2.04 g, 93%, endotherm peak in the DSC at 153.7° C.) as a 1:1 mixture of diastereoisomers.

¹H-NMR (300 MHz, CDCl₃) δ 7.39-7.11 (20H, m), 7.04-6.88 (6H, m), 6.69 (1H, d), 6.61 (1H, d), 6.30 (2H, d), 5.77 (2H, s), 5.69 (2H, s), 5.37 (1H, s), 5.16 (1H, s), 4.53 (2H, s), 4.43 (2H, s), 3.97-3.33 (20H, m), 3.13 (3H, s), 3.03 (3H, s), 2.90 (2H, t), 2.79 (2H, t), 2.31 (6H, s).

### Example 32

### 1-((((2-(Decanoyloxy)ethyl)carbamoyl)oxy)methyl)-1-methyl-4-(2-methyl-10H-benzo[b]thieno[2,3-e][1,4]diazepin-4-yl)piperazin-1-ium iodide (Compound 32)

This compound was synthesized via 2-((iodomethoxy)carbonylamino)ethyl decanoate. The final product precipitated from the reaction mixture and was re-triturated from Et₂O to give Compound 32 (0.79 g, 63%, endotherm peak in the DSC at 155° C.).

¹H-NMR (400 MHz, CDCl₃) δ 7.01-6.90 (3H, m), 6.71 (1H, d), 6.33 (1H, s), 5.64 (2H, s), 5.52 (NH), 4.18 (2H, dd), 4.00-3.92 (2H, m), 3.71-3.62 (6H, m), 3.51 (3H, s), 3.42 (2H, dd), 2.36-2.29 (5H, m), 1.62-1.52 (2H, m), 1.31-1.18 (m, 12H), 0.86 (3H, t).

### Example 33

### 1-(((bis(2-acetoxyethyl)carbamoyl)oxy)methyl)-1-methyl-4-(2-methyl-10H-benzo[b]thieno[2,3-e][1,4]diazepin-4-yl)piperazin-1-ium iodide (Compound 33)

This compound was synthesized via 2,2′-((iodomethoxy)carbonylazanediyl)bis(ethane-2,1-diyl) diacetate. The final product precipitated from the reaction mixture to give Compound 33 (1.46 g, 89%, endotherm peak in the DSC at 139.8° C.).

¹H-NMR (300 MHz, DMSO-d₆) δ 7.74 (NH), 6.86-6.77 (3H, m), 6.70-6.65 (1H, m), 6.38 (1H, s), 5.41 (2H, s), 4.08-4.01 (4H, m), 4.87-4.79 (2H, m), 4.61-4.46 (10H, m), 3.17 (3H, s), 2.25 (3H, s), 2.00 (3H, s), 1.96 (3H, s).

### Example 34

### 1-((((2-Acetoxyethyl)carbamoyl)oxy)methyl)-1-methyl-4-(2-methyl-10H-benzo[b]thieno[2,3-e][1,4]diazepin-4-yl)piperazin-1-ium iodide (Compound 34)

This compound was synthesized via 2-((iodomethoxy)carbonylamino)ethyl acetate. The final product precipitated from the reaction mixture to give Compound 34 (1.40 g, 97%, endotherm peak in the DSC at 141.9° C.).

¹H-NMR (300 MHz, DMSO-d₆) δ 8.11 (NH, t), 7.73 (NH, s), 6.88-6.78 (3H, m), 6.70-6.64 (1H, m), 6.38 (1H, s), 5.38 (2H, s), 4.05 (2H, t), 3.85-3.78 (2H, m), 3.58-3.40 (6H, m), 3.29 (2H, t), 3.12 (3H, s), 2.25 (3H, s), 1.98 (3H, s).

### Example 35

### 1-((((Docosyloxy)carbonyl)oxy)methyl)-1-methyl-4-(2-methyl-10H-benzo[b]thieno[2,3-e][1,4]diazepin-4-yl)piperazin-1-ium iodide (Compound 35)

This compound was synthesized via 1-(((docosyloxy)carbonyl)oxy)methyl iodide. The final product precipitated from the reaction mixture to give Compound 35 (2.56 g, 65%).

¹H-NMR (300 MHz, CDCl₃) δ 6.91-7.01 (3H, m), 6.65 (1H, d), 6.37 (1H, s), 5.91 (2H, s), 5.29 (NH), 4.25 (2H, t), 3.95-4.03 (2H, m), 3.81-3.89 (4H, m), 3.68-3.71 (2H, m), 3.56 (3H, s), 2.32 (3H, s), 1.65-1.71 (2H, m), 1.20-1.38 (36H, m), 0.87 (3H, t).

### Example 36

### 1-(((Hexylcarbamoyl)oxy)methyl)-1-methyl-4-(2-methyl-10H-benzo[b]thieno[2,3-e][1,4]diazepin-4-yl)piperazin-1-ium iodide (Compound 36)

This compound was synthesized via iodomethyl hexylcarbamate with CHCl₃/Et₂O as solvent for the quaternization reaction. The final product precipitated from the reaction mixture to give Compound 36 (1.85 g, 65%, endotherm peak in the DSC at 151.6° C.).

¹H-NMR (300 MHz, CDCl₃) δ 7.02-6.90 (3H, m), 6.72-6.60 (2H, m), 6.33 (1H, s), 5.63 (2H, s), 5.41 (NH), 4.02-3.90 (2H, m), 6.78-6.63 (6H, m), 3.52 (3H, s), 3.16 (2H, q), 2.32 (3H, s), 1.57-1.50 (2H, m), 1.32-1.20 (6H, m), 0.87 (3H, t).

### Example 37

### (S)-1-((((1-(benzyloxy)-3-methyl-1-oxobutan-2-yl)carbamoyl)oxy)methyl)-1-methyl-4-(2-methyl-10H-benzo[b]thieno[2,3-e][1,4]diazepin-4-yl)piperazin-1-ium iodide (Compound 37)

This compound was synthesized via (S)-benzyl 2-((iodomethoxy)carbonylamino)-3-methylbutanoate. The quaternization reaction was carried out in ethyl acetate using (S)-benzyl 2-((iodomethoxy)carbonylamino)-3-methylbutanoate and after 4 hours the solvent was decanted from the reaction. The remaining gummy solid was purified by dissolving in a minimum amount of dichloromethane and adding to 10% ethyl acetate/Et₂O to give Compound 37 (1.47 g, 45%).

¹H-NMR (300 MHz, CDCl₃) δ 7.36-7.28 (5H, m), 6.98-6.91 (4H, m), 6.70 (2H, dd), 6.38 (1H, s), 5.72 (2H, s), 5.16 (2H, dd), 4.24-4.19 (1H, m), 4.01-3.84 (2H, m), 3.76-3.53 (6H, m), 3.51 (3H, s), 2.28 (3H, s), 1.27-1.21 (1H, m), 0.96 (6H, t).

### Example 38

### 1-((((carboxymethyl)carbamoyl)oxy)methyl)-1-methyl-4-(2-methyl-10H-benzo[b]thieno[2,3-e][1,4]diazepin-4-yl)piperazin-1-ium chloride (Compound 38)

This compound was synthesized via tert-butyl 2-((iodomethoxy)carbonylamino)acetate. The quaternization reaction was carried out in ethyl acetate, and after 4 hours the reaction mixture was filtered and dried to give Compound 38 as the iodide salt (300 mg, 60%).

¹H-NMR (300 MHz, CDCl₃) δ 7.30 (NH), 7.01-6.93 (3H, m), 6.82-6.76 (1H, m), 6.37 (1H, s), 5.68 (2H, s), 4.03-3.90 (2H, m), 3.88-3.62 (8H, m), 3.48 (3H, s), 2.31 (3H,$), 1.45 (9H, s).

To a solution of 142-tert-butoxy-2-oxoethylcarbamoyloxy)methyl)-1-methyl-4-(2-methyl-10H-benzo[b]thieno[2,3-e][1,4]diazepin-4-yl)piperazin-1-ium iodide (250 mg, 0.40 mmol) in dichloromethane (20 mL) was added 2M HCl/Et₂O (20 mL), a solid began to precipitate instantly. The reaction was stirred for 30 minutes then the reaction mixture left to settle. The solvent was then decanted and further dichloromethane (10 mL) was added and the remaining solid triturated. The solvent was decanted and the remaining solid dried under a stream of argon gas. The solid was then purified by dissolving in a minimum amount of dimethyl formamide (˜2 mL) and then adding dichloromethane (−30 mL). A solid precipitated and the solvent decanted. The remaining solid was then triturated a further 3 times with dichloromethane. The remaining solid was then suspended in dichloromethane and dried using a Genevac (after each 24 hour period the solid was re-suspended in dichloromethane) for 3 days to remove the last of the dimethyl formamide to give Compound 38 as the chloride salt. (208 mg, 23%, contains 5% olanzapine and 1.5% DMF).

¹H-NMR (300 MHz, d₆-DMSO) δ 8.38 (1H, t), 7.92 (2H, s), 7.31-6.88 (4H, m), 6.59 (1H, br s), 5.45 (2H, s), 4.29-2.96 (13H, m), 2.29 (3H, s).

### Example 39

### 1-((((2-(Benzyloxy)-2-oxoethyl)carbamoyl)oxy)methyl)-1-methyl-4-(2-methyl-10H-benzo[b]thieno[2,3-e][1,4]diazepin-4-yl)piperazin-1-ium iodide (Compound 39)

This compound was synthesized via benzyl 2-((iodomethoxy)carbonylamino)acetate. The product precipitated from the reaction and was further purified by dissolving in industrial methylated spirits and dichloromethane (3:1) and precipitating with diethyl ether to give Compound 39 (2.15 g, 69%).

¹H-NMR (400 MHz, CDCl₃) δ 8.46 (1H, m), 7.73 (1H, s), 7.33 (5H, m), 6.81 (2H, m), 6.67 (1H, m), 6.37 (1H, s), 5.41 (2H, s), 5.13 (2H, s), 3.94 (2H, d), 3.78 (2H, m), 3.30-3.53 (6H, m), 3.11 (3H, s), 2.46 (1H, s), 2.25 (3H, s).

### Example 40

### (S)-1-((((1-(ethoxy)-4-methyl-1-oxopentan-2-yl)carbamoyl)oxy)methyl)-1-methyl-4-(2-methyl-10H-benzo[b]thieno[2,3-e][1,4]diazepin-4-yl)piperazin-1-ium iodide (Compound 40)

This compound was synthesized via (S)-ethyl 2-((iodomethoxy)carbonylamino)-4-methylpentanoate. The product precipitated from the reaction and was further purified by dissolving in the minimum volume of dichloromethane followed by precipitation with diethyl ether to give Compound 40 (1.89 g, 60%).

¹H-NMR (300 MHz, CDCl₃) δ 6.98 (3H, m), 6.84 (1H, d), 6.69 (1H, d), 6.35 (1H, s), 5.75 (2H, s), 5.41 (1H, s), 4.15-4.31 (3H, m), 3.96 (2H, m), 3.72 (6H, m), 3.55 (3H, s), 2.31 (3H, s), 1.60-1.81 (3H, m), 1.27 (3H, t), 0.94 (6H, t).

### Example 41

### (S)-1-((((1-(benzyloxy)-4-methyl-1-oxopentan-2-yl)carbamoyl)oxy)methyl)-1-methyl-4-(2-methyl-10H-benzo[b]thieno[2,3-e][1,4]diazepin-4-yl)piperazin-1-ium iodide (Compound 41)

This compound was synthesized via (S)-benzyl 2-((iodomethoxy)carbonylamino)-4-methylpentanoate. The product precipitated from the reaction mixture upon completion by the addition of diethyl ether and was further purified by dissolving in the minimum volume of dichloromethane and precipitating with diethyl ether/ethyl acetate (1:1) to give Compound 41 (0.81 g, 61%).

¹H-NMR (300 MHz, CDCl₃) δ 7.38 (5H, m), 6.91-7.05 (4H, m), 6.69 (1H, d), 6.36 (1H, s), 5.70 (2H, m), 5.46 (1H, br s), 5.14 (2H, s), 4.32 (1H, m), 3.89 (2H, m), 3.56-3.78 (6H, m), 3.47 (3H, s), 2.31 (3H, s), 1.63-1.87 (3H, m), 0.91 (6H, m).

### Example 42

### (S)-1-((((1-ethoxy-3-methyl-1-oxobutan-2-yl)carbamoyl)oxy)methyl)-1-methyl-4-(2-methyl-10H-benzo[b]thieno[2,3-e][1,4]diazepin-4-yl)piperazin-1-ium iodide (Compound 42)

This compound was synthesized via (S)-ethyl 2-((iodomethoxy)carbonylamino)-3-methylbutanoate. The product precipitated from the reaction mixture and was further purified by dissolving in the minimum volume of dichloromethane and precipitating with diethyl ether/ethyl acetate (8:2) to give Compound 42 (1.56 g, 50%).

¹H-NMR (300 MHz, CDCl₃) δ 6.98 (3H, m), 6.73 (1H, d), 6.47 (1H, d), 6.38 (1H, s), 5.75 (2H, m), 5.61 (1H, br s), 4.21 (3H, m), 3.98 (2H, m), 3.64-3.85 (6H, m), 3.55 (3H, s), 2.23-2.18 (4H, m), 1.28 (3H, t), 0.98 (6H, t).

### Example 43

### (S)-1-(((2-((benzyloxy)carbonyl)pyrrolidine-1-carbonyl)oxy)methyl)-1-methyl-4-(2-methyl-10H-benzo[b]thieno[2,3-e][1,4]diazepin-4-yl)piperazin-1-ium iodide (Compound 43)

This compound was synthesized via (S)-2-benzyl 1-iodomethylpyrrolidine-1,2-dicarboxylate The product precipitated from the reaction and was further purified by dissolving in the minimum volume of dichloromethane/acetonitrile (1:1) and precipitating with ethyl acetate to give Compound 43 (0.78 g, 59%). The product exists as a mixture of conformers (3:1) by ¹H-NMR.

¹H-NMR (300 MHz, CDCl₃) δ 7.39 (5H, m), 6.97 (3H, m), 6.65 (1H, t), 6.31 (1H, s), 5.96 (1H, d), 5.67 (1H, d), 5.19 (3H, m), 4.58 (0.75H, dd), 4.44 (0.25H, dd), 3.27-4.04 (10.75H, m), 2.98 (2.25H, s), 2.31-2.38 (4H, m), 1.80-2.23 (3H, m).

### Example 44

### (S)-1-((((1-(benzyloxy)-1-oxo-3-phenylpropan-2-yl)carbamoyl)oxy)methyl)-1-methyl-4-(2-methyl-10H-benzo[b]thieno[2,3-e][1,4]diazepin-4-yl)piperazin-1-ium iodide (Compound 44)

This compound was synthesized via (S)-benzyl 2-((iodomethoxy)carbonylamino)-3-phenylpropanoate employing general procedure IV and the product precipitated from the reaction. This was further purified by dissolving in the minimum volume of dichloromethane/acetonitrile (1:1) and precipitating with diethyl ether/ethyl acetate to give Compound 44 (0.43 g, 20%).

¹H-NMR (300 MHz, DMSO-d₆) δ 8.63 (NH, d), 7.75 (NH, bs), 7.15-7.38 (10H, m), 6.80-6.93 (3H, m), 6.65-6.75 (1H, m), 5.28-5.39 (2H, m), 5.13 (2H, s), 4.40-4.50 (1H, m), 3.40-3.80 (6H, m), 3.16 (1H, dd), 3.01 (3H, s), 2.83-2.93 (1H, m), 2.27 (3H, s).

### Example 45

### (S)-1-((((1-(benzyloxy)-1-oxopropan-2-yl)carbamoyl)oxy)methyl)-1-methyl-4-(2-methyl-10H-benzo[b]thieno[2,3-e][1,4]diazepin-4-yl)piperazin-1-ium iodide (Compound 45)

This compound was synthesized via (S)-benzyl 2-((iodomethoxy)carbonylamino)propanoate. The product precipitated from the reaction and was further purified by dissolving in the minimum volume of dichloromethane and precipitating with diethyl ether/ethyl acetate to give Compound 45 (1.75 g, 59%).

¹H-NMR (300 MHz, DMSO-d₆) δ 8.53 (NH, d), 7.74 (NH, s), 7.30-7.36 (5H, m), 6.79-6.89 (3H, m), 6.65-6.70 (1H, m), 6.37 (1H, s), 5.35-5.43 (2H, m), 5.13 (2H, s), 4.16-4.23 (1H, m), 3.72-3.83 (2H, m), 3.35-3.55 (6H, m), 3.10 (3H, s), 2.23 (3H, s), 1.34 (3H, d).

### Compound 46

### (S)-1-((((1-(ethoxy)-1-oxopropan-2-yl)carbamoyl)oxy)methyl)-1-methyl-4-(2-methyl-10H-benzo[b]thieno[2,3-e][1,4]diazepin-4-yl)piperazin-1-ium iodide (Compound 46)

This compound was synthesized via (S)-ethyl 2-((iodomethoxy)carbonylamino)propanoate. The product precipitated from the reaction and was further purified by dissolving in the minimum volume of dichloromethane and precipitating with diethyl ether/ethyl acetate to give Compound 46 (1.33 g, 48%).

¹H-NMR (300 MHz, DMSO-d₆) δ 8.49 (NH, d), 7.74 (1H, s), 6.79-6.86 (3H, m), 6.65-6.70 (1H, m), 6.37 (1H, s), 5.39-5.42 (2H, m), 4.10 (2H, q), 3.75-3.90 (2H, m), 3.40-3.60 (6H, m), 3.13 (3H, s), 2.26 (3H, s), 1.31 (3H, d), 1.16 (3H, t).

### Example 47

### 1-((((2-ethoxy-2-oxoethyl)carbamoyl)oxy)methyl)-1-methyl-4-(2-methyl-10H-benzo[b]thieno[2,3-e][1,4]diazepin-4-yl)piperazin-1-ium iodide (Compound 47)

This compound was synthesized via ethyl 2-((iodomethoxy)carbonylamino)acetate. The product precipitated from the reaction and was further purified by dissolving in the minimum volume of acetonitrile and precipitating with diethyl ether to give Compound 47 (1.62 g, 56%).

¹H-NMR (300 MHz, DMSO-d₆) δ 8.43 (NH, t), 7.74 (NH, s), 6.79-6.89 (3H, m), 6.65-6.71 (1H, m), 6.38 (2H, s), 4.10 (2H, q), 7.78-7.89 (4H, m), 3.42-3.60 (6H, m), 3.13 (3H, s), 2.25 (3H, s), 1.17 (3H, t).

### Example 48

### (S)-1-((((1-ethoxy-1-oxo-3-phenylpropan-2-yl)carbamoyl)oxy)methyl)-1-methyl-4-(2-methyl-10H-benzo[b]thieno[2,3-e][1,4]diazepin-4-yl)piperazin-1-ium iodide (Compound 48)

This compound was synthesized via (S)-ethyl 2-((iodomethoxy)carbonylamino)-3-phenylpropanoate. The product precipitated from the reaction and was further purified by dissolving in the minimum volume of acetonitrile and precipitating with ethyl acetate to give Compound 48 (1.73 g, 52%).

¹H-NMR (300 MHz, DMSO-d₆) δ 8.57 (NH, d), 7.75 (NH, 1H), 7.20-7.28 (4H, m), 7.13-7.20 (1H, m), 7.80-7.88 (3H, m), 6.65-7.00 (1H, m), 6.36 (1H, s), 5.28-5.38 (2H, m), 4.31-4.40 (1H, m), 4.09 (2H, q), 3.65-3.83 (2H, m), 3.25-3.55 (6H, m), 3.12 (1H, dd), 3.02 (3H, s), 2.88 (1H, dd), 2.27 (3H, s), 1.13 (3H, t).

### Example 49

### 1-((((2-(benzyloxy)-2-oxoethyl)(methyl)carbamoyl)oxy)methyl)-1-methyl-4-(2-methyl-10H-benzo[b]thieno[2,3-e][1,4]diazepin-4-yl)piperazin-1-ium iodide (Compound 49)

This compound was synthesized via benzyl 2-(((iodomethoxy)carbonyl)(methyl)amino)acetate. The product precipitated from the reaction to give Compound 49 (0.28 g, 50%).

¹H-NMR (300 MHz, CDCl₃) δ 7.42-7.31 (5H, m), 7.03-6.90 (3H, m), 6.72-6.66 (1H, m), 6.33 (1H, s), 5.86 (2H, s), 5.17 (2H, s), 4.09 (2H, s), 4.00-3.85 (2H, m), 3.78-3.45 (6H, m), 3.05 (3H, s), 2.31 (3H, s).

### Example 50

### 1-methyl-4-(2-methyl-10H-benzo[b]thieno[2,3-e][1,4]diazepin-4-yl)-1-(((2-octyldecanoyl)oxy)methyl)piperazin-1-ium iodide (Compound 50)

### Step A—Synthesis of Diethyl 2,2-dioctylmalonate

To a solution of diethylmalonate (20 g, 0.125 mol) in tetrahydrofuran (500 mL) was added octyl bromide (47 mL, 0.275 mol), followed by sodium hydride (60% in mineral oil, 11 g, 0.275 mol) over 1 h. The reaction mixture was stirred at 25° C. for 3 days. A second portion of sodium hydride (5 g, 0.125 mol) and octyl bromide (15 mL, 0.086) were added and the mixture heated at reflux for 5 h. The reaction was cooled, carefully quenched with water and then diluted with 2M HCl. The reaction mixture was extracted with ethyl acetate, dried over MgSO₄ and evaporated. The residue was further purified by flash column chromatography eluting with 1:1 heptane/toluene to toluene to give diethyl 2,2-dioctylmalonate (41.4 g, 86%) as a pale yellow oil.

¹H-NMR (300 MHz, CDCl₃) δ 3.98 (4H, q), 1.70-1.60 (4H, m), 1.15-0.88 (30H, m), 0.69 (6H, t).

### Step B—Synthesis of 2-Octyldecanoic acid

To diethyl 2,2-dioctylmalonate (41.4 g, 0.108 mol) was added industrial methylated spirit (50 mL), followed by a solution of KOH (40 g, 0.714 mol) in water (500 mL). The reaction mixture was heated at reflux for 20 h, poured into ice/water and made acidic with 2M HCl. The mixture was then extracted with ethyl acetate and the organic phase dried over MgSO₄ before evaporation of the volatiles. The residue was then heated neat at 170° C. until gas evolution had ceased (−5 h) and on cooling 2-octyldecanoic acid (26.4 g, 86%) was obtained as a yellow solid.

¹H-NMR (300 MHz, CDCl₃) δ2.40-2.26 (1H, m), 1.66-1.52 (2H, m), 1.51-1.39 (2H, m), 1.35-1.18 (24H, m), 0.87 (3H, t).

### Step C—Synthesis of Chloromethyl 2-octyldecanoate

To a mixture of 2-octyldecanoic acid (12.2 g, 42.9 mmol) and water (90 mL) was added Na₂CO₃ (17.7 g, 108 mmol), tetrabutylammonium hydrogensulfate (2.8 g, 8.2 mmol), dichloromethane (180 mL) and then chloromethyl chlorosulfate (5.5 mL, 54.3 mmol). The reaction mixture was stirred for 18 h and then diluted with water (300 mL) and dichloromethane (300 mL). The organic phase was separated, dried over MgSO₄ and evaporated. The residue was purified on silica eluting with heptane/dichloromethane (8:1) to give chloromethyl 2-octyldecanoate (12.0 g, 84%) as a colorless oil.

¹H-NMR (300 MHz, CDCl₃) δ 5.72 (2H, s), 2.43-2.33 (1H, m), 1.67-1.52 (2H, m), 1.51-1.40 (2H, m), 1.33-1.18 (24H, m), 0.86 (3H, t).

### Step D—Synthesis of Iodomethyl 2-octyldecanoate

A mixture of chloromethyl 2-octyldecanoate (12.0 g, 0.036 mol), sodium iodide (27 g, 0.18 mol) and acetonitrile (300 mL) was stirred for 48 h. The reaction was concentrated, diluted with water (250 mL) and extracted with ethyl acetate (250 mL). The organic phase was washed with water (200 mL), dried over MgSO₄ and evaporated to give iodomethyl 2-octyldecanoate (13.5 g, 88%) as a light brown oil.

¹H-NMR (300 MHz, CDCl₃) δ 5.91 (2H, s), 2.35-2.29 (1H, m), 1.64-1.52 (2H, m), 1.50-1.38 (2H, m), 1.30-1.18 (24H, m), 0.87 (3H, t).

### Step E—Synthesis of Compound 50

To a solution of olanzapine (5.0 g, 0.016 mol) in ethyl acetate (150 mL) was added iodomethyl 2-octyldecanoate (7.13 g, 0.016 mol) and the mixture stirred for 20 h. The reaction mixture was then filtered, washed with ethyl acetate and dried under vacuum at 40° C. to give Compound 50 (10.2 g, 87%, endotherm peak in the DSC at 165.9° C.) as a yellow solid.

¹H-NMR (300 MHz, CDCl₃) δ 6.99-6.89 (3H, m), 6.82-6.78 (1H, m), 6.38 (1H, s), 5.78 (2H, s), 5.47 (NH), 3.99-3.87 (2H, m), 3.82-3.70 (6H, m), 3.55 (3H, s), 2.50 (1H, q), 2.30 (3H, s), 1.68-1.42 (4H, m), 1.31-1.18 (24H, m), 0.87 (3H, t).

### Example 51

### 1-(((2-butylhexanoyl)oxy)methyl)-1-methyl-4-(2-methyl-10H-benzo[b]thieno[2,3-e][1,4]diazepin-4-yl)piperazin-1-ium iodide (Compound 52)

This compound was synthesized according to the general method of Example 50 via iodomethyl 2-butylhexanoate. The product precipitated from the reaction to give Compound 51 (1.44 g, 72%, endotherm peak in the DSC at 193° C.).

¹H-NMR (300 MHz, CDCl₃) δ 6.96 (3H, m), 6.66 (1H, d), 6.36 (1H, s), 5.81 (2H, s), 5.30 (1H, s), 3.98 (2H, m), 3.78 (6H, m), 3.56 (3H, s), 2.50 (1H, m), 2.31 (3H, s), 1.49-1.72 (4H, m), 1.27-1.35 (8H, m), 0.87 (6H, t).

### Example 52

### 1-(((2-hexyloctanoyl)oxy)methyl)-1-methyl-4-(2-methyl-10H-benzo[b]thieno[2,3-e][1,4]diazepin-4-yl)piperazin-1-ium iodide (Compound 52)

This compound was synthesized according to the general method of Example 50 via iodomethyl 2-hexyloctanoate. The product precipitated from the reaction to give Compound 52 (1.31 g, 75%, endotherm peak in the DSC at 177.5° C.).

¹H-NMR (300 MHz, CDCl₃) δ 6.97 (3H, m), 6.63 (1H, d), 6.35 (1H, s), 5.84 (2H, s), 5.18 (1H, s), 3.97 (2H, m), 3.79 (6H, m), 3.56 (3H, s), 2.52 (1H, m), 2.32 (3H, s), 1.60 (4H, m), 1.25 (16H, m), 0.88 (6H, t).

### Example 53

### 1-(((2-decyldodecanoyl)oxy)methyl)-1-methyl-4-(2-methyl-10H-benzo[b]thieno[2,3-e][1,4]diazepin-4-yl)piperazin-1-ium iodide (Compound 53)

This compound was synthesized according to the general method of Example 51 via iodomethyl 2-decyldodecanoate. The product precipitated from the reaction to give Compound 53 (2.33 g, 61%).

¹H-NMR (300 MHz, DMSO-d₆) δ 7.73 (NH, s), 6.78-6.85 (3H, m), 6.65-6.70 (1H, m), 5.44 (2H, s), 3.79-3.88 (2H, m), 3.48-3.60 (6H, m), 3.18 (3H, s), 2.49-2.55 (1H, m), 2.25 (3H, s), 1.40-1.61 (4H, m), 1.12-1.28 (32H, m), 0.81 (6H, t).

### Example 54

### 1-(((((1,3-bis(decanoyloxy)propan-2-yl)oxy)carbonyl)oxy)methyl)-1-methyl-4-(2-methyl-10H-benzo[b]thieno[2,3-e][1,4]diazepin-4-yl)piperazin-1-ium iodide (Compound 54)

### Synthesis of 2-Hydroxypropane-1,3-diyl bis(decanoate)

To a solution of glycerol (2.5 g, 27.14 mmol) in pyridine (50 mL) was added decanoyl chloride (10.6 mL, 51.57 mmol) at 0° C. The reaction was allowed to warm to 25° C. overnight. The reaction was quenched with MeOH (3 mL) before diluting with 2M HCl (50 mL). The reaction was extracted with ethyl acetate (150 mL). The organics were washed with 2M HCl (2×30 mL), brine (30 mL), dried over MgSO₄ and concentrated. A portion of the crude material (2.2 g) was purified by column chromatography eluting with heptane to 40% ethyl acetate in heptane to give 2-hydroxypropane-1,3-diylbis(decanoate) (1.19 g, 10%).

¹H-NMR (400 MHz, CDCl₃) δ 4.14 (5H, m), 2.43 (1H, s), 2.34 (4H, t), 1.52-1.68 (4H, m), 1.27 (24H, m), 0.87 (6H, t).

### Synthesis of 2-((Chloromethoxy)carbonyloxy)propane-1,3-diyl bis(decanoate)

To a solution of 2-hydroxypropane-1,3-diyl bis(decanoate) (1.19 g, 2.97 mmol) in dichloromethane (20 mL) was added pyridine (0.72 mL, 8.91 mmol). The reaction was cooled to 0° C. and chloromethyl chloroformate (0.29 mL, 3.26 mmol) was added slowly. The reaction was allowed to warm to 25° C. after 30 minutes and left overnight. The reaction was incomplete so a catalytic amount of dimethylaminopyridine was added with a further equivalent of chloromethyl chloroformate (0.26 mL, 2.97 mmol) and the reaction left for 24 hours. The reaction was quenched with aqueous sodium hydrogen carbonate solution (20 mL) and extracted with dichloromethane (3×20 mL). The organic phases were washed with aqueous sodium hydrogen carbonate solution (20 mL), 2M HCl (20 mL), brine, dried over MgSO₄ and concentrated. The material was purified by column chromatography and eluted with heptane to 20% ethyl acetate in heptane to give 2-((chloromethoxy)carbonyloxy)propane-1,3-diyl bis(decanoate) (0.543 g, 37%). The product contains 15% of isomer 3-((chloromethoxy)carbonyloxy)propane-1,2-diylbis(decanoate). This could not be removed by chromatography and was carried through to the final product. This was then converted to 2-((iodomethoxy)carbonyloxy)propane-1,3-diyl bis(decanoate) using the general method of Example 1, step C.

¹H-NMR (300 MHz, CDCl₃) δ 5.73 (2H, s), 5.18 (1H, m), 4.36 (2H, dd), 4.18 (2H, dd), 2.32 (4H, t), 1.56-1.62 (4H, m), 1.25 (24H, m), 0.87 (6H, t).

To a solution of olanzapine (0.22 g, 0.70 mmol) in a mixture of ethyl acetate (5 mL) and diethyl ether (2 mL) was added 2-((iodomethoxy)carbonyloxy)propane-1,3-diyl bis(decanoate) (0.45 g, 0.77 mmol). The reaction was stirred at 25° C. for 2 days before addition of a further 0.1 equivalents of 2-((iodomethoxy)carbonyloxy)propane-1,3-diyl bis(decanoate) (0.033 g) to the reaction. The reaction was left for a further 6 days before the product was isolated by filtration. The product was washed with diethyl ether and dried under vacuum to give Compound 54 (0.179 g, 30%). Contains 5% of Compound 55 by ¹H NMR.

¹H-NMR (300 MHz, CDCl₃) δ 6.97 (3H, m), 6.65 (1H, d), 6.36 (1H, s), 6.01 (2H, s), 5.24 (1H, s), 5.01 (1H, m), 4.57 (2H, dd), 4.10 (2H, dd), 3.71-4.07 (8H, m), 3.53 (3H, s), 2.34 (7H, m), 1.59 (4H, m), 1.25 (24H, m), 0.86 (6H, t).

### Example 55

### (S)-1-((((2,3-bis(decanoyloxy)propoxy)carbonyl)oxy)methyl)-1-methyl-4-(2-methyl-10H-benzo[b]thieno[2,3-e][1,4]diazepin-4-yl)piperazin-1-ium iodide (Compound 55)

### Synthesis of (S)-4-(benzyloxymethyl)-2,2-dimethyl-1,3-dioxolane

To a suspension of sodium hydride (4.54 g, 113.5 mmol) in tetrahydrofuran (100 mL) and dimethyl formamide (20 mL) at 0° C. was added a solution of (S)-(+)-2,3-O-isopropylideneglycerol (10 g, 75.7 mmol) in tetrahydrofuran (10 mL) and dimethyl formamide (10 mL) dropwise over 30 minutes. Stirring ceased after addition therefore additional tetrahydrofuran (50 mL) and dimethyl formamide (10 mL) was added. After 1 hour, benzyl bromide (10 mL, 83.2 mmol) was added dropwise over 10 minutes. The reaction was then warmed to 25° C. After 4 hours the reaction was quenched with aq satd NH₄Cl (100 mL) and extracted with ethyl acetate (2×100 mL). The combined organic phases were washed with water (5×100 mL) then brine (100 mL) then dried (MgSO₄) and concentrated to give (S)-4-(benzyloxymethyl)-2,2-dimethyl-1,3-dioxolane (23.6 g) which was used without further purification.

¹H-NMR (300 MHz, CDCl₃) δ 7.43-7.26 (5H, m), 4.57 (2H, dd), 4.35-4.27 (1H, m), 4.09-4.01 (1H, m), 3.76-3.69 (1H, m), 3.55 (1H, dd), 3.45 (1H, dd), 1.42 (3H, s), 1.36 (3H, s).

### Synthesis of (R)-3-(benzyloxy)propane-1,2-diol

(S)-4-(benzyloxymethyl)-2,2-dimethyl-1,3-dioxolane (23.6 g, 106.2 mmol) was stirred in methanol (100 mL) and 2 M HCl (50 mL) and heated to a gentle reflux. After 4 hours the reaction was cooled to 25° C. then aq satd NaHCO₃ was added until pH 7. This was then extracted with dichloromethane (3×250 mL). The combined organic phases were dried (MgSO₄) and concentrated. The crude product was purified using silica column chromatography eluting with dichloromethane to 10% methanol/dichloromethane to give (R)-3-(benzyloxy)propane-1,2-diol (7.81 g, 57%).

¹H-NMR (300 MHz, CDCl₃) δ 7.38-7.26 (5H, m), 4.53 (2H, s), 3.93-3.86 (1H, m), 3.73-3.51 (4H, m), 2.04 (2H, br s).

### Synthesis of (S)-3-(benzyloxy)propane-1,2-diylbis(decanoate)

To a solution of (S)-1-(benzyloxy)ethane-1,2-diol (2.6 g, 14.3 mmol) in dichloromethane (50 mL) at 0° C. was added pyridine (2.9 mL, 35.7 mmol) and decanoyl chloride (6.8 mL, 32.8 mmol). The reaction was gradually warmed to 25° C. and stirred for 5 days. The reaction was quenched with water (50 mL) then separated. The aqueous was extracted with dichloromethane (50 mL). The combined organic phases were washed with water (100 mL), 1M HCl (2×75 mL) and water (100 mL) then dried (MgSO₄) and concentrated. The crude product was purified by silica column chromatography eluting with heptane to 5% ethyl acetate/heptane to give (S)-3-(benzyloxy)propane-1,2-diylbis(decanoate) (6.81 g, 97%).

¹H-NMR (300 MHz, CDCl₃) δ 7.39-7.26 (5H, m), 5.31-5.21 (1H, m), 4.53 (2H, dd), 4.34 (1H, dd), 4.18 (1H, dd), 3.58 (2H, d), 2.37-2.25 (4H, m), 1.64-1.56 (4H, m), 1.37-1.16 (24H, m), 0.87 (6H, t).

### Synthesis of (S)-3-hydroxypropane-1,2-diyl bis(decanoate)

To a solution of (S)-3-(benzyloxy)propane-1,2-diylbis(decanoate) (5.75 g, 11.7 mmol) in ethyl acetate (10 mL) and methanol (10 mL) was added 20% Pd(OH)₂ (0.5 g). The reaction was then stirred at 25° C. under 1 atm of H₂ gas overnight then filtered through celite eluting with ethyl acetate. The organic phase was concentrated and the crude product purified by silica column chromatography eluting with heptane to 20% ethyl acetate/heptane to give (S)-3-hydroxypropane-1,2-diylbis(decanoate) (5.25 g). The product contained impurities but was taken onto the next step without further purification.

¹H-NMR (300 MHz, CDCl₃) δ 5.11-5.05 (1H, m), 4.27 (2H, ddd), 3.73 (2H, d), 2.39-2.29 (4H, m), 1.69-1.51 (4H, m), 1.38-1.14 (24H, m), 0.87 (6H, t).

### Synthesis of (R)-3-((chloromethoxy)carbonyloxy)propane-1,2-diylbis(decanoate)

To a solution of (S)-3-hydroxypropane-1,2-diyl bis(decanoate) (5.1 g, 12.7 mmol) in dichloromethane (100 mL) was added pyridine (3.09 mL, 38.2 mmol). The reaction was cooled to 0° C. and chloromethyl chloroformate (1.24 mL, 14.0 mmol) was added slowly. The reaction was allowed to warm to 25° C. after 30 minutes. After two hours the reaction was incomplete so a further equivalent of chloromethyl chloroformate (1.13 mL, 12.7 mmol) was added and the reaction left for a further three hours. The reaction was quenched with aqueous sodium hydrogen carbonate solution (50 mL) and extracted with dichloromethane (100 mL). The organic phases were washed with aqueous sodium hydrogen carbonate solution (2×30 mL), brine (30 mL), dried over MgSO₄ and concentrated. The product was purified by column chromatography, eluting with heptane to 20% ethyl acetate/heptane to give (R)-3-((chloromethoxy)carbonyloxy)propane-1,2-diylbis(decanoate) (5.35 g, 85%). This was then converted to (R)-3-((iodomethoxy)carbonyloxy)propane-1,2-diylbis(decanoate) using the method of Example 1, step C.

¹H-NMR (400 MHz, CDCl₃) δ 5.71 (2H, m), 5.27 (1H, m), 4.43 (1H, dd), 4.30 (2H, m), 4.14 (1H, dd), 2.30 (4H, m), 1.60 (4H, m), 1.27 (24H, m), 0.86 (6H, t).

To a solution of olanzapine (0.49 g, 1.57 mmol) in a mixture of ethyl acetate (10 mL) and diethyl ether (5 mL) was added (R)-3-((iodomethoxy)carbonyloxy)propane-1,2-diyl bis(decanoate) (0.12 g, 2.03 mmol). The reaction was stirred at 25° C. for 6 days before the product was isolated by filtration. The product was washed with diethyl ether and dried under vacuum to give Compound 55 (0.673 g, 51%).

¹H-NMR (300 MHz, CDCl₃) δ 6.97 (3H, m), 6.67 (1H, s), 6.37 (1H, s), 6.03 (1H, d), 5.93 (1H, d), 5.33 (2H, m), 4.51 (1H, dd), 4.13-4.31 (3H, m), 3.68-4.11 (8H, m), 3.55 (3H, s), 2.33 (7H, m), 1.57 (4H, m), 1.25 (24H, m), 0.87 (6H, t).

### Example 56

### Ammonium (1-methyl-4-(2-methyl-10H-benzo[b]thieno[2,3-e][1,4]diazepin-4-yl)piperazin-1-ium-1-yl)methyl phosphate (Compound 56) and tert-butyl ((1-methyl-4-(2-methyl-10H-benzo[b]thieno[2,3-e][1,4]diazepin-4-yl)piperazin-1-ium-1-yl)methyl) phosphate (Compound 57)

To an ice cold solution of di-tert-butyl phosphate (7.84 g, 40.37 mmol) and KHCO₃ (2.42 g, 24.17 mmol) in H₂O (35 mL), was added KMnO₄ (4.46 g, 28.22 mmol) in three portions. The reaction was allowed to warm to 25° C. and stir for 30 minutes. To the reaction was added charcoal (0.6 g) and the reaction was heated to 60° C. for 15 minutes. The reaction was allowed to cool before filtering through a pad of celite. The celite was washed with H₂O (×3) before the filtrates were combined, stirred with charcoal (1 g) and heated to 60° C. for a further 20 minutes. The reaction was allowed to cool and filtered through a pad of celite. The filtrate was cooled to 0° C. and acidified with conc. HCl (7 mL). The resulting precipitate was isolated by filtration, washed with ice cold H₂O and dissolved in acetone (100 mL). To this was added 10% solution of NMe₄OH (4.38 g in 43 mL of H₂O) at 0° C. The resulting solution was concentrated under vacuum to give tetramethylammonium di-tert-butyl phosphate as a brown oil (6 g).

To a solution of tetramethylammonium di-tert-butyl phosphate (3.6 g, 12.74 mmol) in dimethoxyethane (70 mL) at reflux was added chloroiodomethane (10.2 mL, 140.09 mmol). The reaction was heated for 1.5 hours before allowing to cool to 25° C. The reaction was filtered and the filtrate concentrated under vacuum. The product was purified by column chromatography, eluted 0 to 30% ethyl acetate in heptane to give di-tert-butyl chloromethyl phosphate (1.24 g, 38%).

¹H-NMR (300 MHz, CDCl₃) δ 5.63 (2H, d), 1.48 (18H, s).

To a solution of olanzapine (0.710 g, 2.27 mmol) in acetonitrile (40 mL) was added sodium iodide (0.613 g, 4.09 mmol) followed by di-tert-butyl chloromethyl phosphate (0.823 g, 3.18 mmol). The flask was wrapped in tin foil to eliminate light and the reaction was stirred at room temperature for 3 days. The reaction was concentrated to remove the volatiles before diluting with dichloromethane (30 mL) and washing with H₂O (3×15 mL). The organic phases were passed through a phase separation cartridge and concentrated under vacuum. The resulting oil was stirred in diethyl ether overnight to give 1-((di-tert-butoxyphosphoryloxy)methyl)-1-methyl-4-(2-methyl-10H-benzo[b]thieno[2,3-e][1,4]diazepin-4-yl)piperazin-1-ium iodide as a fine yellow powder (1.105 g). Upon attempted purification by trituration, deprotection occurred leading to the isolation of Compound 56 (0.60 g) as a yellow solid. m/z 479 [M-I⁻].

To Compound 56 (0.383 g, 0.71 mmol) was added trifluoroacetic acid (6 mL). The reaction was stirred at 25° C. for 1.5 hours. To the reaction was added an excess of diethyl ether which resulted in the precipitation of the product. This was filtered and basified via the slow addition of NaHCO₃ solution before purifying under basic preparative HPLC conditions to give Compound 57 (0.227 g, 72%) as a yellow solid.

¹H-NMR (300 MHz, CD₃OD) δ 6.86-6.91 (3H, m), 6.64 (1H, m), 6.42 (1H, s), 4.93 (2H, d), 3.92 (2H, m), 3.64 (4H, m), 3.42 (2H, m), 3.15 (3H, s), 2.30 (3H, s).

### Example 57

### (S)-1-((((1-(docosyloxy)-1-oxopropan-2-yl)carbamoyl)oxy)methyl)-1-methyl-4-(2-methyl-10H-benzo[b]thieno[2,3-e][1,4]diazepin-4-yl)piperazin-1-ium iodide (Compound 58)

### Synthesis of (S)-docosyl 2-(tert-butoxycarbonylamino)propanoate

To a solution of N-Boc-L-alanine (2.5 g, 13.21 mmol) in THF (130 mL) was added 1,1′-carbonyldiimidazole (2.14 g, 13.21 mmol) portionwise. The reaction was heated to 40° C. for 4 hours. To the reaction was added docosanol (4.3 g, 13.21 mmol) and N,N′-dimethylaminopyridine (0.80 g, 6.60 mmol). The reaction was heated at 40° C. overnight before heating to reflux for 20 hours. The reaction was allowed to cool before quenching with saturated NaHCO₃ solution (100 mL) and extracting with ethyl acetate (3×80 mL). The organic phases were combined, washed with brine (50 mL), dried over MgSO₄ and concentrated. The residue was taken up in ethyl acetate and upon standing docosanol precipitated from the solution. This was filtered off and the filtrate concentrated. The material was purified by column chromatography eluting with 0 to 10% ethyl acetate in toluene to give (S)-docosyl 2-(tert-butoxycarbonylamino)propanoate (5.64 g, 86%).

¹H-NMR (300 MHz, CDCl₃) δ 5.05 (1H, br s), 4.31 (1H, m), 4.11 (2H, m), 1.63 (2H, m), 1.44 (9H, s), 1.37 (3H, d), 1.24 (38H, m), 0.87 (3H, t).

### Synthesis of (S)-docosyl 2-((chloromethoxy)carbonylamino)propanoate

To (S)-docosyl 2-(tert-butoxycarbonylamino)propanoate (5.60 g, 11.25 mmol) was added trifluoroacetic acid (5 mL). The reaction was stirred at 25° C. overnight before removing the volatiles under vacuum to give (S)-1-(docosyloxy)-1-oxopropan-2-aminium 2,2,2-trifluoroacetate (4.55 g, 79%).

To a suspension of (S)-1-(docosyloxy)-1-oxopropan-2-aminium 2,2,2-trifluoroacetate (4.35 g, 8.50 mmol) in dichloromethane (70 mL) at 0° C. was added chloromethylchloroformate (1.51 mL, 17.00 mmol) dropwise, followed by the dropwise addition of pyridine (2.06 mL, 25.5 mmol). The reaction was allowed to warm to 25° C. over 2 hours before stirring at 25° C. overnight. The reaction was quenched with saturated NaHCO₃ solution (60 mL) and extracted with dichloromethane (3×50 mL). The organic phases were combined, washed with 2M HCl (50 mL), water (50 mL), brine (50 mL) and dried over MgSO₄ before concentrating under vacuum. A portion was purified by column chromatography eluting with 40 to 60% dichloromethane in heptane to give (S)-docosyl 2-((chloromethoxy)carbonylamino)propanoate (0.269 g) as a colourless solid.

¹H-NMR (400 MHz, CDCl₃) δ 5.76 (1H, d), 5.71 (1H, d), 5.51 (1H, m), 4.38 (1H, m), 4.13 (2H, t), 1.64 (2H, t), 1.44 (3H, d), 1.24 (38H, m), 0.86 (3H, t).

### Synthesis of (S)-docosyl 2-((iodomethoxy)carbonylamino)propanoate

To a suspension of (S)-docosyl 2-((chloromethoxy)carbonylamino)propanoate (0.269 g, 0.55 mmol) in a mixture of acetonitrile (10 mL) and dichloromethane (10 mL) was added sodium iodide (0.247 g, 1.65 mmol). The reaction was wrapped in tin foil to exclude light and the reaction stirred at 25° C. for 7 days. The reaction was concentrated to remove the volatiles. To the residue was added H₂O (30 mL) and the product was extracted with dichloromethane (3×15 mL). The organic phases were washed with 5% aq sodium sulfite solution (20 mL), water (20 mL), dried over MgSO₄ and concentrated to give (S)-docosyl 2-((iodomethoxy)carbonylamino)propanoate (0.320 g, 100%) as a white solid. The product was used in the next reaction without further purification.

¹H-NMR (300 MHz, CDCl₃) δ 5.98 (1H, d), 5.94 (1H, d), 5.46 (1H, m), 4.37 (1H, m), 4.14 (2H, t), 1.60 (2H, m), 1.43 (3H, d), 1.24 (38H, m), 0.87 (3H, t).

To a solution of olanzapine (0.14 g, 0.45 mmol) in ethyl acetate (50 mL) was added a solution of (S)-docosyl 2-((iodomethoxy)carbonylamino)propanoate (0.319 g, 0.54 mmol) in dichloromethane (10 mL). The reaction was stirred overnight at 25° C. The product precipitated from solution and was isolated by decanting off the liquors. The residue was triturated with diethyl ether to give Compound 58 as a yellow solid (0.270 g, 67%).

¹H-NMR (300 MHz, CDCl₃) δ 6.96-7.12 (3H, m), 6.83 (1H, m), 6.46 (1H, s), 5.69 (2H, s), 4.27 (1H, m), 3.71-4.19 (10H, m), 3.51 (3H, s), 2.31 (3H, s), 1.62 (2H, m), 1.51 (3H, d), 1.24 (38H, m), 0.87 (3H, t).

### Example 58

### Pharmacokinetic Evaluation of Prodrugs in Rats

Animals: Male Sprague-Dawley rats (Charles River Laboratories, Wilmington, Mass.) were obtained. Approximately 24 rats were used in each study. Rats were approximately 350-375 g at time of arrival. Rats were housed 2 per cage with ad libitum chow and water. Environmental conditions in the housing room: 64-67° F., 30% to 70% relative humidity, and 12:12-h light:dark cycle. All experiments were approved by the institutional animal care and use committee.

Test Compounds: An amount of each test compound was suspended in the vehicle indicated in Table 6 to yield a suspension comprising the equivalent of 3 mg olanzapine in 0.3 mL.

Pharmacokinetics study: Rats were dosed IM by means of a 23 gauge, 1 in. needle with 1 cc syringe. 0.3 mL suspension was withdrawn from the vial containing the test compound. The rat was injected in the muscles of the hind limb after anesthesia with isoflourane. Blood samples were collected via a lateral tail vein after brief anesthesia with Isoflurane. A 27½ G needle and 1 cc syringe without an anticoagulant were used for the blood collection. Approximately 250 μL, of whole blood was collected at each sampling time point of 6 hours, 24 hours and 2, 5, 7, 9, 12, 14 days after administration. Approximately 450 μL, of whole blood was collected at sampling time points of 21, 28 and 35 days. Once collected, whole blood was immediately transferred to tubes containing K₂ EDTA, inverted 10-15 times and immediately placed on ice. The tubes were centrifuged for 2 minutes at >14,000×g (11500 RPM using Eppendorf Centrifuge 5417C, F45-30-11 rotor) at 4-8° C. to separate plasma. Plasma samples were transferred to labeled plain tubes (Microtainer®; MFG# BD5962) and stored frozen at <−70° C.

Data Analysis: Drug concentrations in plasma samples were analyzed by liquid chromatography-mass spectroscopy using appropriate parameters for each compound. Half-life, volume of distribution, clearance, maximal concentration, and AUC were calculated by using WinNonlin software, version 5.2 (Pharsight, St. Louis, Mo.).

Results: The results are summarized in Table 7 below, which shows the mean parameters for all rats at day 7.

TABLE 7

| | AUC₀₋ₜ | | | |
|---|---|---|---|---|
| Compound | (ng*day/ | Tₘₐₓ | T_{1/2} | |
| No. | mL) | (day) | (day) | Vehicle |
| Olanzapine | 193 | 0.03 | 0.15 | Solution in 100:1 Captisol: |
| | | | | 1M HCl |
| 13 | 77.3 | 0.3 | 0.9 | 2% CMC in PBS with 0.2% |
| | | | | Tween 20. pH 6.6 |
| 10 | 151.0 | 0.3 | 1.6 | 2% CMC in PBS with 0.2% |
| | | | | Tween 20. pH 6.7 |
| 18 | 143.0 | 2.0 | 1.3 | 2% CMC in PBS with 0.2% |
| | | | | Tween 20. pH 6.8 |
| 2 | 135 | 0.3 | 0.2 | 2% CMC in PBS with 0.2% |
| | | | | Tween 20. pH 6.10 |
| 24 | 147.8 | 0.3 | 1.3 | 2% CMC, 0.2% Tween 20 |
| | | | | PBS buffer at pH 6.73 |
| 11 | 126.0 | 0.3 | 0.6 | 2% CMC, 0.2% Tween 20 |
| | | | | PBS buffer at pH 6.73 |
| 50 | 99.0 | 2.0 | 1.7 | 2% CMC, 0.2% Tween 20 |
| | | | | PBS buffer at pH 6.73 |
| 7 | 60.2 | 1.0 | 4.4 | 2% CMC, 0.2% Tween 20 |
| | | | | PBS buffer at pH 6.73 |
| 20 | 55.0 | 1.0 | 1.6 | 2% CMC, 0.2% Tween 20 |
| | | | | PBS buffer at pH 6.73 |
| 17 | 37.4 | 0.3 | 0.3 | 2% CMC, 0.2% Tween 20 |
| | | | | PBS buffer at pH 6.73 |
| 28 | 192.0 | 0.3 | 2.4 | 2% CMC, 0.2% Tween 20 |
| | | | | PBS buffer at pH 6.73 |
| 36 | 151.0 | 0.04 | 1.9 | 2% CMC, 0.2% Tween 20 |
| | | | | PBS buffer at pH 6.73 |
| 51 | 66.5 | 0.63 | 2.66 | 2% CMC, 0.2% Tween 20 |
| | | | | PBS buffer at pH 6.73 |
| 52 | 52.8 | 4.00 | 5.14 | 2% CMC, 0.2% Tween 20 |
| | | | | PBS buffer at pH 6.73 |
| 22 | 63.1 | 1.83 | 1.11 | 2% CMC, 0.2% Tween 20 |
| | | | | PBS buffer at pH 6.73 |
| 53 | 127 | 2.00 | NA | 2% CMC, 0.2% Tween 20 |
| | | | | PBS buffer at pH 6.73 |
| 30 | 7.06 | 0.20 | ND | 2% CMC, 0.2% Tween 20 |
| | | | | PBS buffer at pH 6.73 |
| 43 | 0.4 | 0.20 | ND | 2% CMC, 0.2% Tween 20 |
| | | | | PBS buffer at pH 6.8 |
| 49 | 14.2 | 0.25 | ND | 2% CMC, 0.2% Tween 20 |
| | | | | PBS buffer at pH 6.8 |

The results show that the prodrug compounds have a longer Tₘₐₓ and/or T_{1/2} than olanzapine. This indicates that these compounds provide delayed release of olanzapine to systemic circulation compared to olanzapine itself.

### Example 59

### Solubility of Olanzapine Base, Olanzapine Pamoate Salt and Compound 18 as a Function of pH

Equilibrium solubility of olanzapine free base, olanzapine pamoate and 1-(((2,2-dimethyltetradecanoyl)oxy)methyl)-1-methyl-4-(2-methyl-10H-benzo[b]thieno[2,3-e][1,4]diazepin-4-yl)piperazin-1-ium iodide (Compound 18; OLZ-DMM-I) was measured in aqueous buffers at room temperature in which the three crystalline materials were suspended and equilibrated to saturation, as evidenced by excess solid in suspension. At pH 4 and 5, 0.1 M citrate buffers were used, while for pH 6, 7 and 8 a set of 0.1M phosphate buffers were used. Each buffer also contained 0.2 M NaCl. No cosolvents or other potentially solubilizing components were included. Buffer preparations were subdivided in order to individually test the solubility of only one material in a given buffer sample. The FIGURE shows the pH dependence of the solubility of olanzapine base (triangles) illustrating a greater than a 1000-fold variation in solubility (low solubility at pH 9 to high aqueous solubility at pH 4), consistent with the drugs' basic character. The solubility of olanzapine pamoate salt (OLZ Pamoate; diamond symbols) is pH dependent with slightly more than a 10-fold variation of solubility across the pH range studied. Compound 18 of the invention (OLZ DMM-I; square symbols, solid line) shows negligible pH dependence of solubility (less than 2-fold) across the pH range of 4-9. The room temperature solubility of Compound 18 is uniformly low in water at between 0.0001 and 0.0002 ug/mL. The FIGURE also shows the concentration of olanzapine formed by decomposition of Compound 18 as a function of pH(OLZ from DMM-I; square symbols, dashed line).

### Example 60

### 1-(((2,2-dimethyltetradecanoyl)oxy)methyl)-1-methyl-4-(2-methyl-10H-benzo[b]thieno[2,3-e][1,4]diazepin-4-yl)piperazin-1-ium chloride

General Procedure for Conversion from the Iodide Salt to the Chloride

The olanzapine prodrug chloride salts were prepared from the corresponding iodide by ion exchange on a polymeric macroreticular resin containing quaternary ammonium groups. As an example, 1-(((2,2-dimethyltetradecanoyl)oxy)methyl)-1-methyl-4-(2-methyl-10H-benzo[b]thieno[2,3-e][1,4]diazepin-4-yl)piperazin-1-ium chloride was obtained by the following procedure: 8 g of Amberlyst A-26 (hydroxide form) were loaded as a suspension in methanol on a glass column and 1% HCl in methanol (50 mL) were passed to obtain the chloride form of the resin. The column was washed with methanol (50 mL), and then a methanol solution of 1-(((2,2-dimethyltetradecanoyl)oxy)methyl)-1-methyl-4-(2-methyl-10H-benzo[b]thieno[2,3-e][1,4]diazepin-4-yl)piperazin-1-ium iodide (Compound 18, 181.9 mg in 10 mL of methanol) was passed through the column and eluted with additional methanol (50 mL). The yellow fractions (˜50 mL) were combined and dried under nitrogen flow at room temperature. The solid was suspended in 2-PrOH (10% solid load) with vortexing and sonication. The suspension was stirred at room temperature for 48 hours and filtered. The collected solid was left to dry under vacuum at room temperature to provide the 1-(((2,2-dimethyltetradecanoyl)oxy)methyl)-1-methyl-4-(2-methyl-10H-benzo[b]thieno[2,3-e][1,4]diazepin-4-yl)piperazin-1-ium chloride salt characterized by the endotherm peak in the DSC at 195° C.

### Example 61

### 1-(((2,2-dimethyloctanoyl)oxy)methyl)-1-methyl-4-(2-methyl-10H-benzo[b]thieno[2,3-e][1,4]diazepin-4-yl)piperazin-1-ium chloride

This compound was prepared according to the general method of Example 60 via 1-(((2,2-dimethyloctanoyl)oxy)methyl)-1-methyl-4-(2-methyl-10H-benzo[b]thieno[2,3-e][1,4]diazepin-4-yl)piperazin-1-ium iodide (Compound 19) to give 1-(((2,2-dimethyloctanoyl)oxy)methyl)-1-methyl-4-(2-methyl-10H-benzo[b]thieno[2,3-e][1,4]diazepin-4-yl)piperazin-1-ium chloride (endotherm peak in the DSC at 201° C.).

### Example 62

### 1-(((2,2-dimethyldecanoyl)oxy)methyl)-1-methyl-4-(2-methyl-10H-benzo[b]thieno[2,3-e][1,4]diazepin-4-yl)piperazin-1-ium chloride

This compound was prepared according to the general method of Example 60 via 1-(((2,2-dimethyldecanoyl)oxy)methyl)-1-methyl-4-(2-methyl-10H-benzo[b]thieno[2,3-e][1,4]diazepin-4-yl)piperazin-1-ium iodide (Compound 20) to give 1-(((2,2-dimethyldecanoyl)oxy)methyl)-1-methyl-4-(2-methyl-10H-benzo[b]thieno[2,3-e][1,4]diazepin-4-yl)piperazin-1-ium chloride (endotherm peak in the DSC at 198° C.).

### Example 63

### 1-(((2,2-dimethyldodecanoyl)oxy)methyl)-1-methyl-4-(2-methyl-10H-benzo[b]thieno[2,3-e][1,4]diazepin-4-yl)piperazin-1-ium chloride

This compound was prepared according to the general method of Example 60 via (((2,2-dimethyldodecanoyl)oxy)methyl)-1-methyl-4-(2-methyl-10H-benzo[b]thieno[2,3-e][1,4]diazepin-4-yl)piperazin-1-ium iodide (Compound 21) to give 1-(((2,2-dimethyldodecanoyl)oxy)methyl)-1-methyl-4-(2-methyl-10H-benzo[b]thieno[2,3-e][1,4]diazepin-4-yl)piperazin-1-ium chloride (endotherm peak in the DSC at 199° C.).

### Example 64

### 1-(((2,2-dimethylhexadecanoyl)oxy)methyl)-1-methyl-4-(2-methyl-10H-benzo[b]thieno[2,3-e][1,4]diazepin-4-yl)piperazin-1-ium chloride

This compound was prepared according to the general method of Example 60 via 1-(((2,2-dimethylhexadecanoyl)oxy)methyl)-1-methyl-4-(2-methyl-10H-benzo[b]thieno[2,3-e][1,4]diazepin-4-yl)piperazin-1-ium iodide (Compound 22) to give 1-(((2,2-dimethylhexadecanoyl)oxy)methyl)-1-methyl-4-(2-methyl-10H-benzo[b]thieno[2,3-e][1,4]diazepin-4-yl)piperazin-1-ium chloride (endotherm peak in the DSC at 192° C.).

### Example 65

### 1-methyl-4-(2-methyl-10H-benzo[b]thieno[2,3-e][1,4]diazepin-4-yl)-1-((palmitoyloxy)methyl)piperazin-1-ium chloride

This compound was prepared according to the general method of Example 60 via 1-methyl-4-(2-methyl-10H-benzo[b]thieno[2,3-e][1,4]diazepin-4-yl)-1-((palmitoyloxy)methyl)piperazin-1-ium iodide (Compound 2) to give 1-methyl-4-(2-methyl-10H-benzo[b]thieno[2,3-e][1,4]diazepin-4-yl)-1-((palmitoyloxy)methyl)piperazin-1-ium chloride (endotherm peak in the DSC at 185° C.).

### Example 66

### 1-(((stearoyl)oxy)methyl)-1-methyl-4-(2-methyl-10H-benzo[b]thieno[2,3-e][1,4]diazepin-4-yl)piperazin-1-ium chloride

This compound was prepared according to the general method of Example 60 via 1-(((stearoyl)oxy)methyl)-1-methyl-4-(2-methyl-10H-benzo[b]thieno[2,3-e][1,4]diazepin-4-yl)piperazin-1-ium iodide (Compound 1) to give 1-(((stearoyl)oxy)methyl)-1-methyl-4-(2-methyl-10H-benzo[b]thieno[2,3-e][1,4]diazepin-4-yl)piperazin-1-ium chloride (endotherm peak in the DSC at 185° C.).

### Example 67

### 1-((butyryloxy)methyl)-1-methyl-4-(2-methyl-10H-benzo[b]thieno[2,3-e][1,4]diazepin-4-yl)piperazin-1-ium chloride

This compound was prepared according to the general method of Example 60 via 1-((butyryloxy)methyl)-1-methyl-4-(2-methyl-10H-benzo[b]thieno[2,3-e][1,4]diazepin-4-yl)piperazin-1-ium iodide (Compound 3) to give 1-((butyryloxy)methyl)-1-methyl-4-(2-methyl-10H-benzo[b]thieno[2,3-e][1,4]diazepin-4-yl)piperazin-1-ium chloride (endotherm peak in the DSC at 222° C.).

### Example 68

### 1-methyl-4-(2-methyl-10H-benzo[b]thieno[2,3-e][1,4]diazepin-4-yl)-1-((tetradecanoyloxy)methyl)piperazin-1-ium chloride

This compound was prepared according to the general method of Example 60 via 1-methyl-4-(2-methyl-10H-benzo[b]thieno[2,3-e][1,4]diazepin-4-yl)-1-((tetradecanoyloxy)methyl)piperazin-1-ium iodide (Compound 4) to give 1-methyl-4-(2-methyl-10H-benzo[b]thieno[2,3-e][1,4]diazepin-4-yl)-1-((tetradecanoyloxy)methyl)piperazin-1-ium chloride (endotherm peak in the DSC at 191° C.).

### Example 69

### 1-((dodecanoyloxy)methyl)-1-methyl-4-(2-methyl-10H-benzo[b]thieno[2,3-e][1,4]diazepin-4-yl)piperazin-1-ium chloride

This compound was prepared according to the general method of Example 60 via 1-((dodecanoyloxy)methyl)-1-methyl-4-(2-methyl-10H-benzo[b]thieno[2,3-e][1,4]diazepin-4-yl)piperazin-1-ium iodide (Compound 5) to give 1-((dodecanoyloxy)methyl)-1-methyl-4-(2-methyl-10H-benzo[b]thieno[2,3-e][1,4]diazepin-4-yl)piperazin-1-ium chloride (endotherm peak in the DSC at 180° C.).

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It should also be understood that the embodiments described herein are not mutually exclusive and that features from the various embodiments may be combined in whole or in part in accordance with the invention.

1. A compound of Formula I or Formula II, 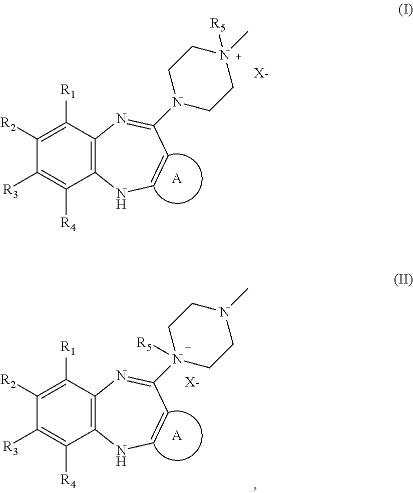
wherein:
Ring A is an optionally substituted fused aryl or heteroaryl ring;
each of R₁ to R₄ is independently selected from hydrogen, halogen, —OR₆, —SR₆, —N(R₆)(R₄), optionally substituted aliphatic, optionally substituted aryl, optionally substituted heteroaryl and optionally substituted heterocyclyl;
R₅ is selected from —C(R₈)(R₉)—OR₁₀, —C(R₈)(R₉)—OC(O)OR₁₀, —C(R₈)(R₉)—OC(O)R₁₀, —C(R₈)(R₉)—OC(O)NR₁₁R₁₂, —C(R₈)(R₉)—OPO₃MY, and —C(R₈)(R₉)—OP(O)(OR₁₁)(OR₁₂);
R₈ and R₉ are each independently hydrogen, aliphatic or substituted aliphatic;
R₁₀ is C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, C₃-C₁₂-cycloalkenyl, substituted C₃-C₁₂-cycloalkenyl, aryl or substituted aryl;
R₁₁ and R₁₂ are each independently hydrogen, aliphatic or substituted aliphatic,
provided that at least one of R₁₁ and R₁₂ is C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₂₄ cycloalkyl, substituted C₃-C₁₂-cycloalkyl, C₃-C₁₂-cycloalkenyl or substituted C₃-C₁₂-cycloalkenyl;
Y and M are the same or different and each is a monovalent cation;
or M and Y together are a divalent cation; and
X⁻ is a pharmaceutically acceptable anion;
or a pharmaceutically acceptable salt thereof.

2. The compound of claim 1 wherein R₅ is selected from —C(R₈)(R₉)—OC(O)OR₁₀, —C(R₈)(R₉)—OC(O)R₁₀, and —C(R₈)(R₉)—OC(O)NR₁₁R₁₂.

3. The compound of claim 1 wherein R₅ is selected from —C(R₈)(R₉)—OPO₃²⁻, —C(R₈)(R₉)—OPO₃MY, and —C(R₈)(R₉)—OP(O)(OR₉)(OR₁₀).

4. The compound of claim 2 wherein R₉ is hydrogen and R₈ is selected from the group consisting of hydrogen and C₁-C₃-alkyl.

5. The compound of claim 2 wherein R₁₀ is C₈-C₂₄-alkyl.

6. The compound of claim 3 wherein R₅ is —C(R₈)(R₉)—OPO₃MY and M and Y together are Ca²⁺.

7. A compound of claim 1 represented by Formula VII, Formula VIII, Formula IX or Formula X:

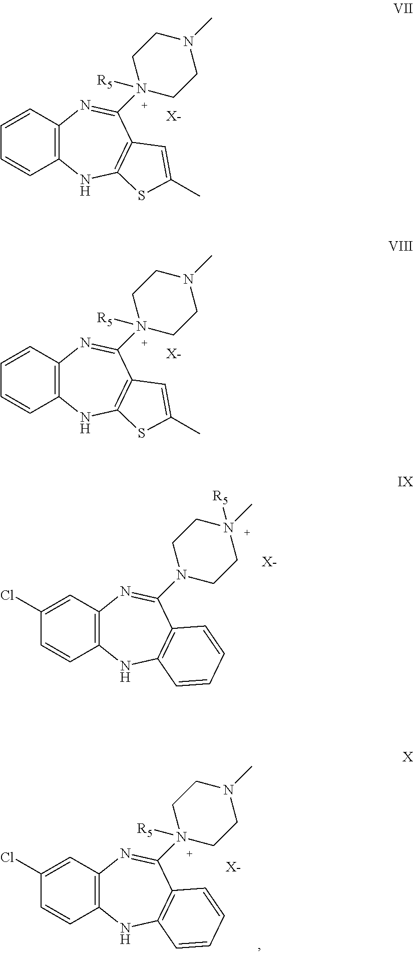

8. The compound of claim 1 wherein R₅ is selected from —CH(R₈)—OC(O)OR₁₀, —CH(R₈)—OC(O)R₁₀ and —CH(R₈)—OC(O)NR₁₁R₁₂.

9. The compound of claim 1 wherein R₅ is selected from —CH(R₈)—OPO₃²⁻, —CH(R₈)—OPO₃MY, and —CH(R₈)—OP(O)(OR₁₁)(OR₁₂).

10. The compound of claim 8 wherein R₈ is selected from the group consisting of hydrogen and C₁-C₃-alkyl.

11. The compound of claim 8 wherein R₁₀ is C₈-C₂₄-alkyl.

12. The compound of claim 9 wherein R₅ is —CH(R₈)—OPO₃MY and M and Y together are Ca²⁺.

13. The compound of claim 1 wherein R₅ is selected from the structures set forth below. 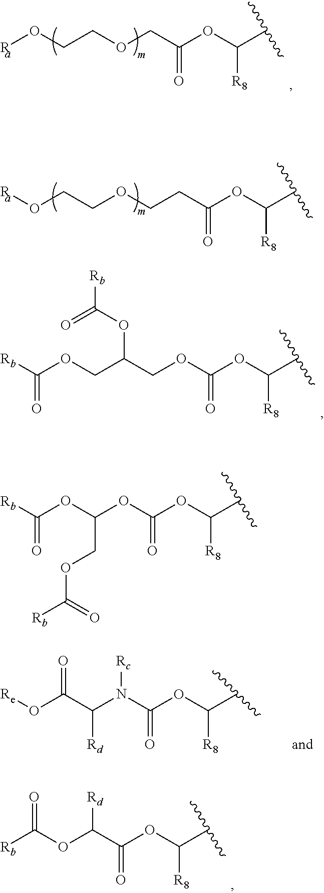
wherein
m is 1 to about 1000;
Rₐ, R_{b} and Rₑ are each independently C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, aryl or substituted aryl;
R_{c} is H or substituted or unsubstituted C₁-C₆-alkyl;
R_{d} is H, substituted or unsubstituted C₁-C₆-alkyl, substituted or unsubstituted aryl-C₁-C₆-alkyl or substituted or unsubstituted heteroaryl-C₁-C₆-alkyl; and
R₈ is as defined in claim 1.

14. A compound of Formula III or Formula IV: 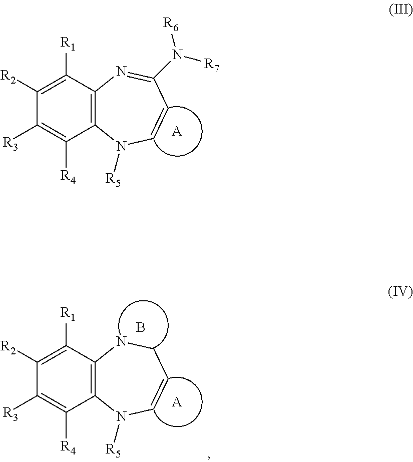
wherein:
Ring A is a fused aryl or heteroaryl ring;
Ring B is a fused heteroaryl ring;
each of R₁ to R₄ is independently selected from hydrogen, halogen, —OR₆, —SR₆, —N(R₆)(R₄), optionally substituted aliphatic, optionally substituted aryl, optionally substituted heteroaryl and optionally substituted heterocyclyl;
R₅ is selected from —C(R₈)(R₉)—OR₁₀, —C(R₈)(R₉)—OC(O)OR₁₀, —C(R₈)(R₉)—OC(O)R₁₀, —C(R₈)(R₉)—OC(O)NR₁₁R₁₂, —C(R₈)(R₉)—OPO₃MY, —C(R₈)(R₉)—OP(O)(OR₁₁)(OR₁₂), —CH(R₈)(R₉)—OP(O)₂(OR₁₁)M, —[C(R₈)(R₉)O]ₙ—R₁₀, —[C(R₈)(R₉)O]ₙ—C(O)OR₁₀, —[C(R₈)(R₉)O], —C(O)R₁₀, —[C(R₈)(R₉)O]ₙ—C(O)NR₁₁R₁₂, —[C(R₈)(R₉)O]ₙ—PO₃MY, —[C(R₈)(R₉)O]ₙ—P(O)₂(OR₁₁)M and —[C(R₈)(R₉)O]ₙ—P(O)(ORᵢᵢ)(OR₁₂);
R₆ and R₇ are each independently C₁-C₁₂-alkyl, substituted C₁-C₁₂-alkyl, C₂-C₁₂-alkenyl, substituted C₂-C₁₂-alkenyl, C₂-C₁₂-alkynyl, substituted C₂-C₁₂-alkynyl, C₃-C₁₂ cycloalkyl, substituted C₃-C₁₂-cycloalkyl; or
R₆, R₇ and the nitrogen atom to which they are attached form a substituted or unsubstituted heterocyclic ring;
R₈ and R₉ are each independently hydrogen, aliphatic or substituted aliphatic;
R₁₀ is C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, C₃-C₁₂-cycloalkenyl, substituted C₃-C₁₂-cycloalkenyl, aryl or substituted aryl;
R₁₁ and R₁₂ are each independently hydrogen, aliphatic or substituted aliphatic, provided that at least one of R₁₁ and R₁₂ is C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₂₄ cycloalkyl, substituted C₃-C₁₂-cycloalkyl, C₃-C₁₂-cycloalkenyl or substituted C₃-C₁₂-cycloalkenyl;
Y and M are the same or different and each is a monovalent cation;
or M and Y together are a divalent cation; and
n is 2 to 6;
or a pharmaceutically acceptable salt thereof.

15. The compound of claim 14 wherein Ring A is benzo or thieno.

16. The compound of claim 14 wherein each of R₁, R₂, R₃ and R₄ is hydrogen.

17. The compound of claim 14 wherein R₁, R₃ and R₄ are each hydrogen, and R₂ is chlorine.

18. The compound of claim 14 wherein Ring A is optionally substituted thieno and each of R₁ to R₄ is hydrogen.

19. The compound of claim 14 wherein Ring A is benzo, R₂ is chlorine and R₁, R₃ and R₄ are each hydrogen.

20. The compound of claim 14 wherein R₅ is selected from —CH(R₈)—OC(O)OR₁₀, —CH(R₈)—OC(O)R₁₀ and —CH(R₈)—OC(O)NR₉R₁₀.

21. The compound of claim 14 wherein R₅ is selected from —CH(R₈)—OPO₃MY, and —CH(R₈)—OP(O)(OR₉)(OR₁₀).

22. The compound of claim 20 wherein R₈ is selected from the group consisting of hydrogen and C₁-C₃-alkyl.

23. The compound of claim 20 wherein R₁₀ is C₈-C₂₄-alkyl.

24. The compound of claim 14 wherein R₅ is —C(R₈)(R₉)—OPO₃MY wherein M and Y together are Ca²⁺.

25. A compound of claim 14 represented by Formula V or Formula VI,

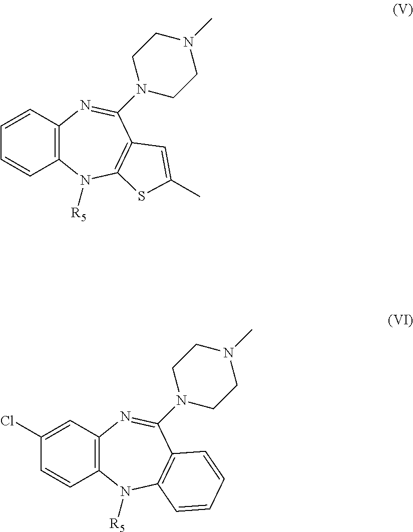

26. The compound of claim 25 wherein R₅ is selected from —C(R₈)(R₉)—OC(O)OR₁₀, —C(R₈)(R₉)—OC(O)R₁₀ and —C(R₈)(R₉)—OC(O)NR₁₁R₁₂.

27. The compound of claim 25 wherein R₅ is selected from —C(R₈)(R₉)—OPO₃²⁻, —C(R₈)(R₉)—OPO₃MY, and —C(R₈)(R₉)—OP(O)(OR₁₁)(OR₁₂).

28. The compound of claim 26 wherein R₈ is selected from the group consisting of hydrogen and C₁-C₃-alkyl.

29. The compound of claim 26 wherein R₁₀ is C₈-C₂₄-alkyl.

30. The compound of claim 27 wherein R₅ is —C(R₈)(R₉)—OPO₃MY and M and Y together are Ca²⁺.

31. The compound of claim 14 wherein R₅ is selected from the structures set forth below. 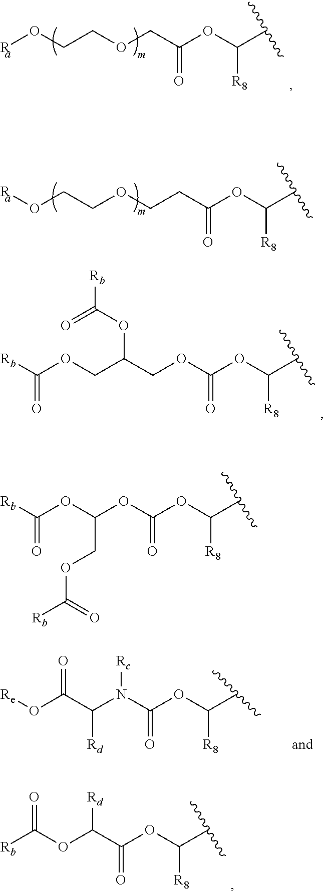
wherein
m is 1 to about 1000;
Rₐ, R_{b} and Rₑ are each independently C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, C₃-C₁₂-cycloalkenyl, substituted C₃-C₁₂-cycloalkenyl. aryl or substituted aryl;
R_{c} is H or substituted or unsubstituted C₁-C₆-alkyl;
R_{d} is H, substituted or unsubstituted C₁-C₆-alkyl, substituted or unsubstituted aryl-C₁-C₆-alkyl or substituted or unsubstituted heteroaryl-C₁-C₆-alkyl; and
R₈ is as defined in claim 1.

32. A method of producing a prodrug of a parent drug, wherein said prodrug has pH-independent solubility properties and wherein the parent drug is represented by the formula below, 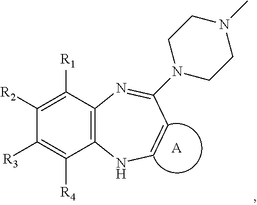
wherein:
Ring A is a fused aryl or heteroaryl ring; and
each of R₁ to R₄ is independently selected from hydrogen, halogen, —OR₆, —SR₆, —N(R₆)(R₄), optionally substituted aliphatic, optionally substituted aryl, optionally substituted heteroaryl and optionally substituted heterocyclyl;
said method comprising the step of reacting the parent drug with a compound represented by
R₅—Y
wherein:
R₅ is selected from —C(R₈)(R₉)—OR₁₀,
R₈ and R₉ are each independently hydrogen, aliphatic or substituted aliphatic;
R₁₀ is C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, C₃-C₁₂-cycloalkenyl, substituted C₃-C₁₂-cycloalkenyl, aryl or substituted aryl; and
Y is a leaving group;
thereby producing a prodrug of the formula 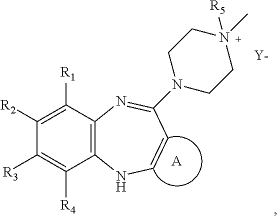
wherein said prodrug has pH-independent solubility properties.

33. A method of treating a neurological or psychiatric disorder in a subject in need thereof, comprising administering to the subject a therapeutically effective amount of a compound of claim 1.

34. The method according to claim 33 wherein said disorder is schizophrenia.

35. The method according to claim 33 wherein the disorder is bipolar disorder.

36. The compound of claim 9 wherein R₈ is s selected from the group consisting of hydrogen and C₁-C₃-alkyl.

37. The compound of claim 27 wherein R₈ is selected from the group consisting of hydrogen and C₁-C₃-alkyl.

38. A method of treating a neurological or psychiatric disorder in a subject in need thereof, comprising administering to the subject a therapeutically effective amount of a compound of claim 14.

39. The method according to claim 3 wherein said disorder is schizophrenia.

40. The method according to claim 38 wherein the disorder is bipolar disorder.
